SEQUENCE LISTING (244 sequences)

SEQ ID NO: 1 - protein, 1013 aa, Homo sapiens
  REGION 1..1013: note = MISC_FEATURE - ELAPOR1/IGFR-L1, UniProtKB Accession Number: Q6UXG2-1
  DOMAIN 1..910: note = Extracellular domain
  SIGNAL 1..41
MAEPGHSHHLSARVRGRTERRIPRLWRLLLWAGTAFQVTQGTGPELHACKESEYHYEYTACDSTGSRWRVAVPHTPGLCTSLPDPIKGTECSFSCNAGEFLDMKDQSCKPCAEGRYSLGTGIRFDEWDELPHGFASLSANMELDDSAAESTGNCTSSKWVPRGDYIASNTDECTATLMYAVNLKQSGTVNFEYYYPDSSIIFEFFVQNDQCQPNADDSRWMKTTEKGWEFHSVELNRGNNVLYWRTTAFSVWTKVPKPVLVRNIAITGVAYTSECFPCKPGTYADKQGSSFCKLCPANSYSNKGETSCHQCDPDKYSEKGSSSCNVRPACTDKDYFYTHTACDANGETQLMYKWAKPKICSEDLEGAVKLPASGVKTHCPPCNPGFFKTNNSTCQPCPYGSYSNGSDCTRCPAGTEPAVGFEYKWWNTLPTNMETTVLSGINFEYKGMTGWEVAGDHIYTAAGASDNDFMILTLVVPGFRPPQSVMADTENKEVARITFVFETLCSVNCELYFMVGVNSRTNTPVETWKGSKGKQSYTYIIEENTTTSFTWAFQRTTFHEASRKYTNDVAKIYSINVTNVMNGVASYCRPCALEASDVGSSCTSCPAGYYIDRDSGTCHSCPTNTILKAHQPYGVQACVPCGPGTKNNKIHSLCYNDCTFSRNTPTRTFNYNFSALANTVTLAGGPSFTSKGLKYFHHFTLSLCGNQGRKMSVCTDNVTDLRIPEGESGFSKSITAYVCQAVIIPPEVTGYKAGVSSQPVSLADRLIGVTTDMTLDGITSPAELFHLESLGIPDVIFFYRSNDVTQSCSSGRSTTIRVRCSPQKTVPGSLLLPGTCSDGTCDGCNFHFLWESAAACPLCSVADYHAIVSSCVAGIQKTTYVWREPKLCSGGISLPEQRVTICKTIDFWLKVGISAGTCTAILLTVLTCYFWKKNQKLEYKYSKLVMNATLKDCDLPAADSCAIMEGEDVEDDLIFTSKKSLFGKIKSFTSKRTPDGFDSVPLKTSSGGLDMDL

SEQ ID NO: 2 - protein, 469 aa, synthetic construct
  REGION 1..469: note = Heavy chain (Hc) of the anti-IGFR-L1 antibody 1 (AB-1)
  REGION 1..18: note = MISC_FEATURE - leader sequence (L)
  REGION 19..48: note = MISC_FEATURE - Framework region HFR1
  REGION 49..54: note = MISC_FEATURE - Complementarity-determining region CDR-H1
  REGION 55..68: note = MISC_FEATURE - Framework region HFR2
  REGION 69..84: note = MISC_FEATURE - Complementarity-determining region CDR-H2
  REGION 85..116: note = MISC_FEATURE - Framework region HFR3
  REGION 117..129: note = MISC_FEATURE - Complementarity-determining region CDR-H3
  REGION 130..140: note = MISC_FEATURE - Framework region HFR4
  REGION 141..469: note = MISC_FEATURE - Tail region
MRMLSLLYLLTALPGILSEIQLQESGPGLVKPSQSLSLTCSVTDYTITSGFGWSWIRKFPGNKLEWMGYISYSGSTNYNPSLKSRLSLTRDTTKNQFFLQLISVTPEDTATYYCARSPLYGSNRYVVDAWGQGASVTVSSARTTAPSVYPLVPGCSGTSGSLVTLGCLVKGYFPEPVTVKWNSGALSSGVHTFPAVLQSGLYTLSSSVTVPSSTWSSQTVTCSVAHPATKSNLIKRIEPRRPKPRPPTDICSCDDNLGRPSVFIFPPKPKDILMITLTPKVTCVVVDVSEEEPDVQFSWFVDNVRVFTAQTQPHEEQLNGTFRVVSTLHIQHQDWMSGKEFKCKVNNKDLPSPIEKTISKPRGKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK

SEQ ID NO: 3 - protein, 18 aa, synthetic construct
  REGION 1..18: note = Leader (L) of the heavy chain (Hc) of the anti-IGFR-L1 antibody 1(AB-1)
MRMLSLLYLLTALPGILS

SEQ ID NO: 4 - protein, 30 aa, synthetic construct
  REGION 1..30: note = HFR1 of the heavy chain (Hc) of the anti-IGFR-L1 antibody 1(AB-1)
EIQLQESGPGLVKPSQSLSLTCSVTDYTIT

SEQ ID NO: 5 - protein, 6 aa, synthetic construct
  REGION 1..6: note = CDR-H1 of the heavy chain (Hc) of the anti-IGFR-L1 antibody 1(AB-1)
SGFGWS

SEQ ID NO: 6 - protein, 14 aa, synthetic construct
  REGION 1..14: note = HFR2 of the heavy chain (Hc) of the anti-IGFR-L1 antibody 1(AB-1)
WIRKFPGNKLEWMG

SEQ ID NO: 7 - protein, 16 aa, synthetic construct
  REGION 1..16: note = CDR-H2 of the heavy chain (Hc) of the anti-IGFR-L1 antibody 1(AB-1)
YISYSGSTNYNPSLKS

SEQ ID NO: 8 - protein, 32 aa, synthetic construct
  REGION 1..32: note = HFR3 of the heavy chain (Hc) of the anti-IGFR-L1 antibody 1(AB-1)
RLSLTRDTTKNQFFLQLISVTPEDTATYYCAR

SEQ ID NO: 9 - protein, 13 aa, synthetic construct
  REGION 1..13: note = CDR-H3 of the heavy chain (Hc) of the anti-IGFR-L1 antibody 1(AB-1)
SPLYGSNRYVVDA

SEQ ID NO: 10 - protein, 11 aa, synthetic construct
  REGION 1..11: note = HFR4 of the heavy chain (Hc) of the anti-IGFR-L1 antibody 1(AB-1)
WGQGASVTVSS

SEQ ID NO: 11 - protein, 329 aa, synthetic construct
  REGION 1..329: note = Tail of the heavy chain (Hc) of the anti-IGFR-L1 antibody 1(AB-1)
ARTTAPSVYPLVPGCSGTSGSLVTLGCLVKGYFPEPVTVKWNSGALSSGVHTFPAVLQSGLYTLSSSVTVPSSTWSSQTVTCSVAHPATKSNLIKRIEPRRPKPRPPTDICSCDDNLGRPSVFIFPPKPKDILMITLTPKVTCVVVDVSEEEPDVQFSWFVDNVRVFTAQTQPHEEQLNGTFRVVSTLHIQHQDWMSGKEFKCKVNNKDLPSPIEKTISKPRGKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK

SEQ ID NO: 12 - protein, 241 aa, synthetic construct
  REGION 1..241: note = Light chain (Lc) of the anti-IGFR-L1 antibody 1 (AB-1)
  REGION 1..27: note = MISC_FEATURE - Leader sequence (L)
  REGION 28..50: note = MISC_FEATURE - Framework region LFR1
  REGION 51..61: note = MISC_FEATURE - Complementarity-determining region CDR-L1
  REGION 62..76: note = MISC_FEATURE - Framework region LFR2
  REGION 77..83: note = MISC_FEATURE - Complementarity-determining region CDR-L2
  REGION 84..115: note = MISC_FEATURE - Framework region LFR3
  REGION 116..124: note = MISC_FEATURE - Complementarity-determining region CDR-L3
  REGION 125..136: note = MISC_FEATURE - Framework region LFR4
  REGION 137..241: note = MISC_FEATURE - Tail region
MSGHNLDMRVQIQFWGLLLLWTSGIQCDVQMTQSPSILTASPGESVSINCKASKSISNYLAWYQQKPGKSINLLIYSGSTLQSGTPSRFSGSGSGTDFTLTIRNLDPEDFGRYYCQQHNENPYTFGPGTRLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 13 - protein, 27 aa, synthetic construct
  REGION 1..27: note = Leader (L) of the light chain (Lc) of the anti-IGFR-L1 antibody 1(AB-1)
MSGHNLDMRVQIQFWGLLLLWTSGIQC

SEQ ID NO: 14 - protein, 23 aa, synthetic construct
  REGION 1..23: note = LFR1 of the light chain (Lc) of the anti-IGFR-L1 antibody 1(AB-1)
DVQMTQSPSILTASPGESVSINC

SEQ ID NO: 15 - protein, 11 aa, synthetic construct
  REGION 1..11: note = CDR-L1 of the light chain (Lc) of the anti-IGFR-L1 antibody 1(AB-1)
KASKSISNYLA

SEQ ID NO: 16 - protein, 15 aa, synthetic construct
  REGION 1..15: note = LFR2 of the light chain (Lc) of the anti-IGFR-L1 antibody 1(AB-1)
WYQQKPGKSINLLIY

SEQ ID NO: 17 - protein, 7 aa, synthetic construct
  REGION 1..7: note = CDR-L2 of the light chain (Lc) of the anti-IGFR-L1 antibody 1(AB-1)
SGSTLQS

SEQ ID NO: 18 - protein, 32 aa, synthetic construct
  REGION 1..32: note = LFR3 of the light chain (Lc) of the anti-IGFR-L1 antibody 1(AB-1)
GTPSRFSGSGSGTDFTLTIRNLDPEDFGRYYC

SEQ ID NO: 19 - protein, 9 aa, synthetic construct
  REGION 1..9: note = CDR-L3 of the light chain (Lc) of the anti-IGFR-L1 antibody 1(AB-1)
QQHNENPYT

SEQ ID NO: 20 - protein, 12 aa, synthetic construct
  REGION 1..12: note = LFR4 of the light chain (Lc) of the anti-IGFR-L1 antibody 1(AB-1)
FGPGTRLELKRA

SEQ ID NO: 21 - protein, 105 aa, synthetic construct
  REGION 1..105: note = Tail of the light chain (Lc) of the anti-IGFR-L1 antibody 1(AB-1)
DAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 22 - protein, 264 aa, synthetic construct
  REGION 1..264: note = Heavy chain (Hc) of the anti-IGFR-L1 antibody 2 (AB-2)
  REGION 1..61: note = MISC_FEATURE - Leader sequence (L)
  REGION 62..91: note = MISC_FEATURE - Framework region HFR1
  REGION 92..96: note = MISC_FEATURE - Complementarity-determining region CDR-H1
  REGION 97..110: note = MISC_FEATURE - Framework region HFR2
  REGION 111..127: note = MISC_FEATURE - Complementarity-determining region CDR-H2
  REGION 128..159: note = MISC_FEATURE - Framework region HFR3
  REGION 160..166: note = MISC_FEATURE - Complementarity-determining region CDR-H3
  REGION 167..177: note = MISC_FEATURE - Framework region HFR4
  REGION 178..264: note = MISC_FEATURE - Tail region
FFFSLPGVWGGGGGGGDGGAQSWTLRFSHSVISGAALHRLLTMDIRLTLVFLVLFIKGVQCEVQLVESDGGFVQPGRSLTLSCAASGFTFSDYYMSWVRQAPTKGLEWVATIGYDVSYTNYRDSVKGRFTISRDNAKSILSLQMDSLRSEDTATYYCARHPSYFDYWGQGVMVTVSSAQTTAPSVYPLAPGCGDTTSSTVTLGCLVKGYFPEPVTVTWNSGALSSDVHTFPAVLQSGLYTLTSSVTSSTWPSQTVTCNVAHPAT

SEQ ID NO: 23 - protein, 61 aa, synthetic construct
  REGION 1..61: note = Leader (L) of the heavy chain (Hc) of the anti-IGFR-L1 antibody 2(AB-2)
FFFSLPGVWGGGGGGGDGGAQSWTLRFSHSVISGAALHRLLTMDIRLTLVFLVLFIKGVQC

SEQ ID NO: 24 - protein, 30 aa, synthetic construct
  REGION 1..30: note = HFR1 of the heavy chain (Hc) of the anti-IGFR-L1 antibody 2(AB-2)
EVQLVESDGGFVQPGRSLTLSCAASGFTFS

SEQ ID NO: 25 - protein, 5 aa, synthetic construct
  REGION 1..5: note = CDR-H1 of the heavy chain (Hc) of the anti-IGFR-L1 antibody 2(AB-2)
DYYMS

SEQ ID NO: 26 - protein, 14 aa, synthetic construct
  REGION 1..14: note = HFR2 of the heavy chain (Hc) of the anti-IGFR-L1 antibody 2(AB-2)
WVRQAPTKGLEWVA

SEQ ID NO: 27 - protein, 17 aa, synthetic construct
  REGION 1..17: note = CDR-H2 of the heavy chain (Hc) of the anti-IGFR-L1 antibody 2(AB-2)
TIGYDVSYTNYRDSVKG

SEQ ID NO: 28 - protein, 32 aa, synthetic construct
  REGION 1..32: note = HFR3 of the heavy chain (Hc) of the anti-IGFR-L1 antibody 2(AB-2)
RFTISRDNAKSILSLQMDSLRSEDTATYYCAR

SEQ ID NO: 29 - protein, 7 aa, synthetic construct
  REGION 1..7: note = CDR-H3 of the heavy chain (Hc) of the anti-IGFR-L1 antibody 2(AB-2)
HPSYFDY

SEQ ID NO: 30 - protein, 11 aa, synthetic construct
  REGION 1..11: note = HFR4 of the heavy chain (Hc) of the anti-IGFR-L1 antibody 2(AB-2)
WGQGVMVTVSS

SEQ ID NO: 31 - protein, 87 aa, synthetic construct
  REGION 1..87: note = Tail of the heavy chain (Hc) of the anti-IGFR-L1 antibody 2(AB-2)
AQTTAPSVYPLAPGCGDTTSSTVTLGCLVKGYFPEPVTVTWNSGALSSDVHTFPAVLQSGLYTLTSSVTSSTWPSQTVTCNVAHPAT

SEQ ID NO: 32 - protein, 258 aa, synthetic construct
  REGION 1..258: note = Light chain (Lc) of the anti-IGFR-L1 antibody 2 (AB-2)
  REGION 1..47: note = MISC_FEATURE - Leader sequence (L)
  REGION 48..70: note = MISC_FEATURE - Framework region LFR1
  REGION 71..87: note = MISC_FEATURE - Complementarity-determining region CDR-L1
  REGION 88..102: note = MISC_FEATURE - Framework region LFR2
  REGION 103..109: note = MISC_FEATURE - Complementarity-determining region CDR-L2
  REGION 110..141: note = MISC_FEATURE - Framework region LFR3
  REGION 142..150: note = MISC_FEATURE - Complementarity-determining region CDR-L3
  REGION 151..162: note = MISC_FEATURE - Framework region LFR4
  REGION 163..258: note = MISC_FEATURE - Tail region
FLSFLRLAPGGGGGGGGEGHQTDRGSKMESQTQVLMSLLLWISGTCGDIVMTQSPSSLAVSAGETVTINCKSSQSLFYSENQKNYLAWLQQKPGQSPKLLITWASTRQSGVPDRFIGSGSGTDFTLTISSVQAEDLAIYHCQQYYDTPRTFGGGTQLELKRADAAPTVSIFPPSTEQLATGGASVVCLMNNFYPRDISVKWKIDGTERRDGVLDSVTDQDSKDSTYSMSSTLSLTKADYESHNLYTCEVVHKTFIPST

SEQ ID NO: 33 - protein, 47 aa, synthetic construct
  REGION 1..47: note = Leader (L) of the light chain (Lc) of the anti-IGFR-L1 antibody 2(AB-2)
FLSFLRLAPGGGGGGGGEGHQTDRGSKMESQTQVLMSLLLWISGTCG

SEQ ID NO: 34 - protein, 23 aa, synthetic construct
  REGION 1..23: note = LFR1 of the light chain (Lc) of the anti-IGFR-L1 antibody 2(AB-2)
DIVMTQSPSSLAVSAGETVTINC

SEQ ID NO: 35 - protein, 17 aa, synthetic construct
  REGION 1..17: note = CDR-L1 of the light chain (Lc) of the anti-IGFR-L1 antibody 2(AB-2)
KSSQSLFYSENQKNYLA

SEQ ID NO: 36 - protein, 15 aa, synthetic construct
  REGION 1..15: note = LFR2 of the light chain (Lc) of the anti-IGFR-L1 antibody 2(AB-2)
WLQQKPGQSPKLLIT

SEQ ID NO: 37 - protein, 7 aa, synthetic construct
  REGION 1..7: note = CDR-L2 of the light chain (Lc) of the anti-IGFR-L1 antibody 2(AB-2)
WASTRQS

SEQ ID NO: 38 - protein, 32 aa, synthetic construct
  REGION 1..32: note = LFR3 of the light chain (Lc) of the anti-IGFR-L1 antibody 2(AB-2)
GVPDRFIGSGSGTDFTLTISSVQAEDLAIYHC

SEQ ID NO: 39 - protein, 9 aa, synthetic construct
  REGION 1..9: note = CDR-L3 of the light chain (Lc) of the anti-IGFR-L1 antibody 2(AB-2)
QQYYDTPRT

SEQ ID NO: 40 - protein, 12 aa, synthetic construct
  REGION 1..12: note = LFR4 of the light chain (Lc) of the anti-IGFR-L1 antibody 2(AB-2)
FGGGTQLELKRA

SEQ ID NO: 41 - protein, 96 aa, synthetic construct
  REGION 1..96: note = Tail of the light chain (Lc) of the anti-IGFR-L1 antibody 2(AB-2)
DAAPTVSIFPPSTEQLATGGASVVCLMNNFYPRDISVKWKIDGTERRDGVLDSVTDQDSKDSTYSMSSTLSLTKADYESHNLYTCEVVHKTFIPST

SEQ ID NO: 42 - protein, 465 aa, synthetic construct
  REGION 1..465: note = Heavy chain (Hc) of the anti-IGFR-L1 antibody 3 (AB-3)
  REGION 1..19: note = MISC_FEATURE - Leader sequence (L)
  REGION 20..49: note = MISC_FEATURE - Framework region HFR1
  REGION 50..54: note = MISC_FEATURE - Complementarity-determining region CDR-H1
  REGION 55..68: note = MISC_FEATURE - Framework region HFR2
  REGION 69..87: note = MISC_FEATURE - Complementarity-determining region CDR-H2
  REGION 88..119: note = MISC_FEATURE - Framework region HFR3
  REGION 120..125: note = MISC_FEATURE - Complementarity-determining region CDR-H3
  REGION 126..136: note = MISC_FEATURE - Framework region HFR4
  REGION 137..465: note = MISC_FEATURE - Tail region
MKLWLNWIFLSTLLNGIQCEVQLLESGGGLVQPGGSLSLSCAASGFTFTDFYMNWIRQPAGKAPEWLAFARNKANGYTTDYNPSVKGRFAISRDNTQNMFYLHMNTLRPEDTATYYCARGRPSDYWGQGVMVTVSSARTTAPSVYPLVPGCSGTSGSLVTLGCLVKGYFPEPVTVKWNSGALSSGVHTFPAVLQSGLYTLSSSVTVPSSTWSSQTVTCSVAHPATKSNLIKRIEPRRPKPRPPTDICSCDDNLGRPSVFIFPPKPKDILMITLTPKVTCVVVDVSEEEPDVQFSWFVDNVRVFTAQTQPHEEQLNGTFRVVSTLHIQHQDWMSGKEFKCKVNNKDLPSPIEKTISKPRGKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK

SEQ ID NO: 43 - protein, 19 aa, synthetic construct
  REGION 1..19: note = Leader (L) of the heavy chain (Hc) of the anti-IGFR-L1 antibody 3(AB-3)
MKLWLNWIFLSTLLNGIQC

SEQ ID NO: 44 - protein, 30 aa, synthetic construct
  REGION 1..30: note = HFR1 of the heavy chain (Hc) of the anti-IGFR-L1 antibody 3(AB-3)
EVQLLESGGGLVQPGGSLSLSCAASGFTFT

SEQ ID NO: 45 - protein, 5 aa, synthetic construct
  REGION 1..5: note = CDR-H1 of the heavy chain (Hc) of the anti-IGFR-L1 antibody 3(AB-3)
DFYMN

SEQ ID NO: 46 - protein, 14 aa, synthetic construct
  REGION 1..14: note = HFR2 of the heavy chain (Hc) of the anti-IGFR-L1 antibody 3(AB-3)
WIRQPAGKAPEWLA

SEQ ID NO: 47 - protein, 19 aa, synthetic construct
  REGION 1..19: note = CDR-H2 of the heavy chain (Hc) of the anti-IGFR-L1 antibody 3(AB-3)
FARNKANGYTTDYNPSVKG

SEQ ID NO: 48 - protein, 32 aa, synthetic construct
  REGION 1..32: note = HFR3 of the heavy chain (Hc) of the anti-IGFR-L1 antibody 3(AB-3)
RFAISRDNTQNMFYLHMNTLRPEDTATYYCAR

SEQ ID NO: 49 - protein, 6 aa, synthetic construct
  REGION 1..6: note = CDR-H3 of the heavy chain (Hc) of the anti-IGFR-L1 antibody 3(AB-3)
GRPSDY

SEQ ID NO: 50 - protein, 11 aa, synthetic construct
  REGION 1..11: note = HFR4 of the heavy chain (Hc) of the anti-IGFR-L1 antibody 3(AB-3)
WGQGVMVTVSS

SEQ ID NO: 51 - protein, 329 aa, synthetic construct; identical to SEQ ID NO: 11
  REGION 1..329: note = Tail of the heavy chain (Hc) of the anti-IGFR-L1 antibody 3(AB-3)

SEQ ID NO: 52 - protein, 246 aa, synthetic construct
  REGION 1..246: note = Light chain (Lc) of the anti-IGFR-L1 antibody 3 (AB-3)
  REGION 1..38: note = MISC_FEATURE - Leader sequence (L)
  REGION 39..61: note = MISC_FEATURE - Framework region LFR1
  REGION 62..78: note = MISC_FEATURE - Complementarity-determining region CDR-L1
  REGION 79..93: note = MISC_FEATURE - Framework region LFR2
  REGION 94..100: note = MISC_FEATURE - Complementarity-determining region CDR-L2
  REGION 101..132: note = MISC_FEATURE - Framework region LFR3
  REGION 133..141: note = MISC_FEATURE - Complementarity-determining region CDR-L3
  REGION 142..153: note = MISC_FEATURE - Framework region LFR4
  REGION 154..246: note = MISC_FEATURE - Tail region
FSGGGGGGGRHQTDRGSKMESQTQVLMSLLLWISGTCGDIVMTQSPSSLAVSTGETVTMDCKSSQNLFYSGNQRNYLAWYQQKPGQSPTLLIYWASTRRSGVPDRFIGSGSGTDFTLTISSVQAEDLALYYCQQYDDPPYTFGAGTKLELKRADAAPTVSIFPPSTEQLATGGASVVCLMNNFYPRDISVKWKIDGTERRDGVLDSVTDQDSKDSTYSMSSTLSLTKADYESHNLYTCEVFIKTLL

SEQ ID NO: 53 - protein, 38 aa, synthetic construct
  REGION 1..38: note = Leader (L) of the light chain (Lc) of the anti-IGFR-L1 antibody 3(AB-3)
FSGGGGGGGRHQTDRGSKMESQTQVLMSLLLWISGTCG

SEQ ID NO: 54 - protein, 23 aa, synthetic construct
  REGION 1..23: note = LFR1 of the light chain (Lc) of the anti-IGFR-L1 antibody 3(AB-3)
DIVMTQSPSSLAVSTGETVTMDC

SEQ ID NO: 55 - protein, 17 aa, synthetic construct
  REGION 1..17: note = CDR-L1 of the light chain (Lc) of the anti-IGFR-L1 antibody 3(AB-3)
KSSQNLFYSGNQRNYLA

SEQ ID NO: 56 - protein, 15 aa, synthetic construct
  REGION 1..15: note = LFR2 of the light chain (Lc) of the anti-IGFR-L1 antibody 3(AB-3)
WYQQKPGQSPTLLIY

SEQ ID NO: 57 - protein, 7 aa, synthetic construct
  REGION 1..7: note = CDR-L2 of the light chain (Lc) of the anti-IGFR-L1 antibody 3(AB-3)
WASTRRS

SEQ ID NO: 58 - protein, 32 aa, synthetic construct
  REGION 1..32: note = LFR3 of the light chain (Lc) of the anti-IGFR-L1 antibody 3(AB-3)
GVPDRFIGSGSGTDFTLTISSVQAEDLALYYC

SEQ ID NO: 59 - protein, 9 aa, synthetic construct
  REGION 1..9: note = CDR-L3 of the light chain (Lc) of the anti-IGFR-L1 antibody 3(AB-3)
QQYDDPPYT

SEQ ID NO: 60 - protein, 12 aa, synthetic construct
  REGION 1..12: note = LFR4 of the light chain (Lc) of the anti-IGFR-L1 antibody 3(AB-3)
FGAGTKLELKRA

SEQ ID NO: 61 - protein, 93 aa, synthetic construct
  REGION 1..93: note = Tail of the light chain (Lc) of the anti-IGFR-L1 antibody 3(AB-3)
DAAPTVSIFPPSTEQLATGGASVVCLMNNFYPRDISVKWKIDGTERRDGVLDSVTDQDSKDSTYSMSSTLSLTKADYESHNLYTCEVFIKTLL

SEQ ID NO: 62 - protein, 467 aa, synthetic construct
  REGION 1..467: note = Heavy chain (Hc) of the anti-IGFR-L1 antibody 4 (AB-4)
  REGION 1..19: note = MISC_FEATURE - Leader sequence (L)
  REGION 20..49: note = MISC_FEATURE - Framework region HFR1
  REGION 50..54: note = MISC_FEATURE - Complementarity-determining region CDR-H1
  REGION 55..68: note = MISC_FEATURE - Framework region HFR2
  REGION 69..84: note = MISC_FEATURE - Complementarity-determining region CDR-H2
  REGION 85..116: note = MISC_FEATURE - Framework region HFR3
  REGION 117..130: note = MISC_FEATURE - Complementarity-determining region CDR-H3
  REGION 131..141: note = MISC_FEATURE - Framework region HFR4
  REGION 142..467: note = MISC_FEATURE - Tail region
MYFSISLFFLVLILKSVQCEVQLVESGGGFVRPGKSLKLSCSASGFTFNTYGMHWIRQAPGKGLDWVSYISSSSATAYADAVKGRFTISRDNAKNTLYLQLNSLRSEDTAIYHCAREATYYGYPRYYFDYWGQGVMVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 63 - protein, 19 aa, synthetic construct
  REGION 1..19: note = Leader (L) of the heavy chain (Hc) of the anti-IGFR-L1 antibody 4(AB-4)
MYFSISLFFLVLILKSVQC

SEQ ID NO: 64 - protein, 30 aa, synthetic construct
  REGION 1..30: note = HFR1 of the heavy chain (Hc) of the anti-IGFR-L1 antibody 4(AB-4)
EVQLVESGGGFVRPGKSLKLSCSASGFTFN

SEQ ID NO: 65 - protein, 5 aa, synthetic construct
  REGION 1..5: note = CDR-H1 of the heavy chain (Hc) of the anti-IGFR-L1 antibody 4(AB-4)
TYGMH

SEQ ID NO: 66 - protein, 14 aa, synthetic construct
  REGION 1..14: note = HFR2 of the heavy chain (Hc) of the anti-IGFR-L1 antibody 4(AB-4)
WIRQAPGKGLDWVS

SEQ ID NO: 67 - protein, 16 aa, synthetic construct
  REGION 1..16: note = CDR-H2 of the heavy chain (Hc) of the anti-IGFR-L1 antibody 4(AB-4)
YISSSSATAYADAVKG

SEQ ID NO: 68 - protein, 32 aa, synthetic construct
  REGION 1..32: note = HFR3 of the heavy chain (Hc) of the anti-IGFR-L1 antibody 4(AB-4)
RFTISRDNAKNTLYLQLNSLRSEDTAIYHCAR

SEQ ID NO: 69 - protein, 14 aa, synthetic construct
  REGION 1..14: note = CDR-H3 of the heavy chain (Hc) of the anti-IGFR-L1 antibody 4(AB-4)
EATYYGYPRYYFDY

SEQ ID NO: 70 - protein, 11 aa, synthetic construct
  REGION 1..11: note = HFR4 of the heavy chain (Hc) of the anti-IGFR-L1 antibody 4(AB-4)
WGQGVMVTVSS

SEQ ID NO: 71 - protein, 326 aa, synthetic construct
  REGION 1..326: note = Tail of the heavy chain (Hc) of the anti-IGFR-L1 antibody 4(AB-4)
AETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 72 - protein, 237 aa, synthetic construct
  REGION 1..237: note = Light chain (Lc) of the anti-IGFR-L1 antibody 4 (AB-4)
  REGION 1..39: note = MISC_FEATURE - Leader sequence (L)
  REGION 40..62: note = MISC_FEATURE - Framework region LFR1
  REGION 63..73: note = MISC_FEATURE - Complementarity-determining region CDR-L1
  REGION 74..88: note = MISC_FEATURE - Framework region LFR2
  REGION 89..95: note = MISC_FEATURE - Complementarity-determining region CDR-L2
  REGION 96..127: note = MISC_FEATURE - Framework region LFR3
  REGION 128..136: note = MISC_FEATURE - Complementarity-determining region CDR-L3
  REGION 137..148: note = MISC_FEATURE - Framework region LFR4
  REGION 149..237: note = MISC_FEATURE - Tail region
RLGRGGGGGLSLQSGLSMDMRAHTQFLGFLWLLFAGARCDIQMTQSPSSMSVSLGDRVTFTCRASQDIGNYLSWYQQKPEKSPRLMIYTATDLEDGVPSRFSGSRSGSDYSLTINSLESEDRGIYFCLQHKQYPYTFGSGTKLELKRADAAPTVSIFPPSTEQLATGGASVVCLMNNFYPRDISVKWKIDGTERRDGVLDSVTDQDSKDSTYSMSSTLSLTKADYESHNLYTCEVGS

SEQ ID NO: 73 - protein, 39 aa, synthetic construct
  REGION 1..39: note = Leader (L) of the light chain (Lc) of the anti-IGFR-L1 antibody 4(AB-4)
RLGRGGGGGLSLQSGLSMDMRAHTQFLGFLWLLFAGARC

SEQ ID NO: 74 - protein, 23 aa, synthetic construct
  REGION 1..23: note = LFR1 of the light chain (Lc) of the anti-IGFR-L1 antibody 4(AB-4)
DIQMTQSPSSMSVSLGDRVTFTC

SEQ ID NO: 75 - protein, 11 aa, synthetic construct
  REGION 1..11: note = CDR-L1 of the light chain (Lc) of the anti-IGFR-L1 antibody 4(AB-4)
RASQDIGNYLS

SEQ ID NO: 76 - protein, 15 aa, synthetic construct
  REGION 1..15: note = LFR2 of the light chain (Lc) of the anti-IGFR-L1 antibody 4(AB-4)
WYQQKPEKSPRLMIY

SEQ ID NO: 77 - protein, 7 aa, synthetic construct
  REGION 1..7: note = CDR-L2 of the light chain (Lc) of the anti-IGFR-L1 antibody 4(AB-4)
TATDLED

SEQ ID NO: 78 - protein, 32 aa, synthetic construct
  REGION 1..32: note = LFR3 of the light chain (Lc) of the anti-IGFR-L1 antibody 4(AB-4)
GVPSRFSGSRSGSDYSLTINSLESEDRGIYFC

SEQ ID NO: 79 - protein, 9 aa, synthetic construct
  REGION 1..9: note = CDR-L3 of the light chain (Lc) of the anti-IGFR-L1 antibody 4(AB-4)
LQHKQYPYT

SEQ ID NO: 80 - protein, 12 aa, synthetic construct
  REGION 1..12: note = LFR4 of the light chain (Lc) of the anti-IGFR-L1 antibody 4(AB-4)
FGSGTKLELKRA

SEQ ID NO: 81 - protein, 89 aa, synthetic construct
  REGION 1..89: note = Tail of the light chain (Lc) of the anti-IGFR-L1 antibody 4(AB-4)
DAAPTVSIFPPSTEQLATGGASVVCLMNNFYPRDISVKWKIDGTERRDGVLDSVTDQDSKDSTYSMSSTLSLTKADYESHNLYTCEVGS

SEQ ID NO: 82 - protein, 263 aa, synthetic construct
  REGION 1..263: note = Heavy chain (Hc) of the anti-IGFR-L1 antibody 5 (AB-5)
  REGION 1..57: note = MISC_FEATURE - Leader sequence (L)
  REGION 58..87: note = MISC_FEATURE - Framework region HFR1
  REGION 88..92: note = MISC_FEATURE - Complementarity-determining region CDR-H1
  REGION 93..106: note = MISC_FEATURE - Framework region HFR2
  REGION 107..123: note = MISC_FEATURE - Complementarity-determining region CDR-H2
  REGION 124..155: note = MISC_FEATURE - Framework region HFR3
  REGION 156..164: note = MISC_FEATURE - Complementarity-determining region CDR-H3
  REGION 165..175: note = MISC_FEATURE - Framework region HFR4
  REGION 176..263: note = MISC_FEATURE - Tail region
SCILGGGGGGGWDAGAQSWTLRFSHSVISTAALHRLLTMDIRLSLAFLVLFTKGVQCEVQLVESGGGLVQPGRSLKLSCAAPGFTFSASDMAWVRQAPTKGLEWVASISPSGDSTYYRDSILGRFTVSRDNAKSSLYLQMDSLRSEDTATYYCARHLGLGPFDHWGQGVMVTVSSAETTAPSVYPLAPGTALKSNSMVTLGCLVKGYFPEPVTVTWNSGALSSGVHTFPAVLQSGLYTLTSSVTVPSSTWPSQTVTCNEPPAK

SEQ ID NO: 83 - protein, 57 aa, synthetic construct
  REGION 1..57: note = Leader (L) of the heavy chain (Hc) of the anti-IGFR-L1 antibody 5(AB-5)
SCILGGGGGGGWDAGAQSWTLRFSHSVISTAALHRLLTMDIRLSLAFLVLFTKGVQC

SEQ ID NO: 84 - protein, 30 aa, synthetic construct
  REGION 1..30: note = HFR1 of the heavy chain (Hc) of the anti-IGFR-L1 antibody 5(AB-5)
EVQLVESGGGLVQPGRSLKLSCAAPGFTFS

SEQ ID NO: 85 - protein, 5 aa, synthetic construct
  REGION 1..5: note = CDR-H1 of the heavy chain (Hc) of the anti-IGFR-L1 antibody 5(AB-5)
ASDMA

SEQ ID NO: 86 - protein, 14 aa, synthetic construct
  REGION 1..14: note = HFR2 of the heavy chain (Hc) of the anti-IGFR-L1 antibody 5(AB-5)
WVRQAPTKGLEWVA

SEQ ID NO: 87 - protein, 17 aa, synthetic construct
  REGION 1..17: note = CDR-H2 of the heavy chain (Hc) of the anti-IGFR-L1 antibody 5(AB-5)
SISPSGDSTYYRDSILG

SEQ ID NO: 88 - protein, 32 aa, synthetic construct
  REGION 1..32: note = HFR3 of the heavy chain (Hc) of the anti-IGFR-L1 antibody 5(AB-5)
RFTVSRDNAKSSLYLQMDSLRSEDTATYYCAR

SEQ ID NO: 89 - protein, 9 aa, synthetic construct
  REGION 1..9: note = CDR-H3 of the heavy chain (Hc) of the anti-IGFR-L1 antibody 5(AB-5)
HLGLGPFDH

SEQ ID NO: 90 - protein, 11 aa, synthetic construct
  REGION 1..11: note = HFR4 of the heavy chain (Hc) of the anti-IGFR-L1 antibody 5(AB-5)
WGQGVMVTVSS

SEQ ID NO: 91 - protein, 88 aa, synthetic construct
  REGION 1..88: note = Tail of the heavy chain (Hc) of the anti-IGFR-L1 antibody 5(AB-5)
AETTAPSVYPLAPGTALKSNSMVTLGCLVKGYFPEPVTVTWNSGALSSGVHTFPAVLQSGLYTLTSSVTVPSSTWPSQTVTCNEPPAK

SEQ ID NO: 92 - protein, 222 aa, synthetic construct
  REGION 1..222: note = Light chain (Lc) of the anti-IGFR-L1 antibody 5 (AB-5)
  REGION 1..20: note = MISC_FEATURE - Leader sequence (L)
  REGION 21..43: note = MISC_FEATURE - Framework region LFR1
  REGION 44..54: note = MISC_FEATURE - Complementarity-determining region CDR-L1
  REGION 55..69: note = MISC_FEATURE - Framework region LFR2
  REGION 70..76: note = MISC_FEATURE - Complementarity-determining region CDR-L2
  REGION 77..108: note = MISC_FEATURE - Framework region LFR3
  REGION 109..117: note = MISC_FEATURE - Complementarity-determining region CDR-L3
  REGION 118..129: note = MISC_FEATURE - Framework region LFR4
  REGION 130..222: note = MISC_FEATURE - Tail region
MMAPVQLLGLLLLWLPAMRCDIQMTQSPSLLSASVGDRVTLSCKAGRDINNYLAWYQQKLGEAPKLLIYNANFLQTGIPSRFSGSGSGTDYTLTISSLQPEDVATYFCQQFNTWTGTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVFIRLFE

SEQ ID NO: 93 - protein, 20 aa, synthetic construct
  REGION 1..20: note = Leader (L) of the light chain (Lc) of the anti-IGFR-L1 antibody 5(AB-5)
MMAPVQLLGLLLLWLPAMRC

SEQ ID NO: 94 - protein, 23 aa, synthetic construct
  REGION 1..23: note = LFR1 of the light chain (Lc) of the anti-IGFR-L1 antibody 5(AB-5)
DIQMTQSPSLLSASVGDRVTLSC

SEQ ID NO: 95 - protein, 11 aa, synthetic construct
  REGION 1..11: note = CDR-L1 of the light chain (Lc) of the anti-IGFR-L1 antibody 5(AB-5)
KAGRDINNYLA

SEQ ID NO: 96 - protein, 15 aa, synthetic construct
  REGION 1..15: note = LFR2 of the light chain (Lc) of the anti-IGFR-L1 antibody 5(AB-5)
WYQQKLGEAPKLLIY

SEQ ID NO: 97 - protein, 7 aa, synthetic construct
  REGION 1..7: note = CDR-L2 of the light chain (Lc) of the anti-IGFR-L1 antibody 5(AB-5)
NANFLQT

SEQ ID NO: 98 - protein, 32 aa, synthetic construct
  REGION 1..32: note = LFR3 of the light chain (Lc) of the anti-IGFR-L1 antibody 5(AB-5)
GIPSRFSGSGSGTDYTLTISSLQPEDVATYFC

SEQ ID NO: 99 - protein, 9 aa, synthetic construct
  REGION 1..9: note = CDR-L3 of the light chain (Lc) of the anti-IGFR-L1 antibody 5(AB-5)
QQFNTWTGT

SEQ ID NO: 100 - protein, 12 aa, synthetic construct
  REGION 1..12: note = LFR4 of the light chain (Lc) of the anti-IGFR-L1 antibody 5(AB-5)
FGGGTKLELKRA

SEQ ID NO: 101 - protein, 93 aa, synthetic construct
  REGION 1..93: note = Tail of the light chain (Lc) of the anti-IGFR-L1 antibody 5(AB-5)
DAAPTVSIFPPSTEQLATGGASVVCLMNNFYPRDISVKWKIDGTERRDGVLDSVTDQDSKDSTYSMSSTLSLTKADYESHNLYTCEVFIRLFE

SEQ ID NO: 102 - protein, 259 aa, synthetic construct
  REGION 1..259: note = Heavy chain (Hc) of the anti-IGFR-L1 antibody 6 (AB-6)
  REGION 1..53: note = MISC_FEATURE - Leader sequence (L)
  REGION 54..83: note = MISC_FEATURE - Framework region HFR1
  REGION 84..88: note = MISC_FEATURE - Complementarity-determining region CDR-H1
  REGION 89..102: note = MISC_FEATURE - Framework region HFR2
  REGION 103..119: note = MISC_FEATURE - Complementarity-determining region CDR-H2
  REGION 120..151: note = MISC_FEATURE - Framework region HFR3
  REGION 152..160: note = MISC_FEATURE - Complementarity-determining region CDR-H3
  REGION 161..171: note = MISC_FEATURE - Framework region HFR4
  REGION 172..259: note = MISC_FEATURE - Tail region
HGGGGGGGDGGAQSWTLRFSHSVISAAALHRLLTMDIRLSLVFLVLFIKGVQCEVQLVESDGGLVQPGRSLKLSCAASGFSFSDYYMAWLRQAPTKGLEWVATISHDGTGTYFRDSVKGRFTISRDNVKSTLYLQLDSLRSEDTATYYCARHKDGGYKDYWGQGVMVTVSSAETTAPSVYPLAPGTALKSNSMVTLGCLVKGYFPEPVTVTWNSGALSSGVHTFPAVLQSGLYTLTSSVTVPSSTWPSQTVTCNEPPAD

SEQ ID NO: 103 - protein, 53 aa, synthetic construct
  REGION 1..53: note = Leader (L) of the heavy chain (Hc) of the anti-IGFR-L1 antibody 6(AB-6)
HGGGGGGGDGGAQSWTLRFSHSVISAAALHRLLTMDIRLSLVFLVLFIKGVQC

SEQ ID NO: 104 - protein, 30 aa, synthetic construct
  REGION 1..30: note = HFR1 of the heavy chain (Hc) of the anti-IGFR-L1 antibody 6(AB-6)
EVQLVESDGGLVQPGRSLKLSCAASGFSFS

SEQ ID NO: 105 - protein, 5 aa, synthetic construct
  REGION 1..5: note = CDR-H1 of the heavy chain (Hc) of the anti-IGFR-L1 antibody 6(AB-6)
DYYMA

SEQ ID NO: 106 - protein, 14 aa, synthetic construct
  REGION 1..14: note = HFR2 of the heavy chain (Hc) of the anti-IGFR-L1 antibody 6(AB-6)
WLRQAPTKGLEWVA

SEQ ID NO: 107 - protein, 17 aa, synthetic construct
  REGION 1..17: note = CDR-H2 of the heavy chain (Hc) of the anti-IGFR-L1 antibody 6(AB-6)
TISHDGTGTYFRDSVKG

SEQ ID NO: 108 - protein, 32 aa, synthetic construct
  REGION 1..32: note = HFR3 of the heavy chain (Hc) of the anti-IGFR-L1 antibody 6(AB-6)
RFTISRDNVKSTLYLQLDSLRSEDTATYYCAR

SEQ ID NO: 109 - protein, 9 aa, synthetic construct
  REGION 1..9: note = CDR-H3 of the heavy chain (Hc) of the anti-IGFR-L1 antibody 6(AB-6)
HKDGGYKDY

SEQ ID NO: 110 - protein, 11 aa, synthetic construct
  REGION 1..11: note = HFR4 of the heavy chain (Hc) of the anti-IGFR-L1 antibody 6(AB-6)
WGQGVMVTVSS

SEQ ID NO: 111 - protein, 88 aa, synthetic construct
  REGION 1..88: note = Tail of the heavy chain (Hc) of the anti-IGFR-L1 antibody 6(AB-6)
AETTAPSVYPLAPGTALKSNSMVTLGCLVKGYFPEPVTVTWNSGALSSGVHTFPAVLQSGLYTLTSSVTVPSSTWPSQTVTCNEPPAD

SEQ ID NO: 112 - protein, 176 aa, synthetic construct
  REGION 1..176: note = Light chain (Lc) of the anti-IGFR-L1 antibody 6 (AB-6)
  REGION 1..54: note = MISC_FEATURE - Leader sequence (L)
  REGION 55..77: note = MISC_FEATURE - Framework region LFR1
  REGION 78..88: note = MISC_FEATURE - Complementarity-determining region CDR-L1
  REGION 89..103: note = MISC_FEATURE - Framework region LFR2
  REGION 104..110: note = MISC_FEATURE - Complementarity-determining region CDR-L2
  REGION 111..142: note = MISC_FEATURE - Framework region LFR3
  REGION 143..150: note = MISC_FEATURE - Complementarity-determining region CDR-L3
  REGION 151..163: note = MISC_FEATURE - Framework region LFR4
  REGION 164..176: note = MISC_FEATURE - Tail region
MDEKKERRRIQRGGAGYIRGWKGRGALQSSLNMDIRAPVQFLGILQLWLLGAQCDIKMTQSPSPLSASLGERVTISCRASDNIDNYLSWYQKKEDGSVRLLIYHTSRLQPGAPSRFSGSGSGKDYSLTISGLESEDIATYYCQQTYTPYTFGAGTKLELRRADAAPTVSISHHHEA

SEQ ID NO: 113 - protein, 54 aa, synthetic construct
  REGION 1..54: note = Leader (L) of the light chain (Lc) of the anti-IGFR-L1 antibody 6(AB-6)
MDEKKERRRIQRGGAGYIRGWKGRGALQSSLNMDIRAPVQFLGILQLWLLGAQC

SEQ ID NO: 114 - protein, 23 aa, synthetic construct
  REGION 1..23: note = LFR1 of the light chain (Lc) of the anti-IGFR-L1 antibody 6(AB-6)
DIKMTQSPSPLSASLGERVTISC

SEQ ID NO: 115 - protein, 11 aa, synthetic construct
  REGION 1..11: note = CDR-L1 of the light chain (Lc) of the anti-IGFR-L1 antibody 6(AB-6)
RASDNIDNYLS

SEQ ID NO: 116 - protein, 15 aa, synthetic construct
  REGION 1..15: note = LFR2 of the light chain (Lc) of the anti-IGFR-L1 antibody 6(AB-6)
WYQKKEDGSVRLLIY

SEQ ID NO: 117 - protein, 7 aa, synthetic construct
  REGION 1..7: note = CDR-L2 of the light chain (Lc) of the anti-IGFR-L1 antibody 6(AB-6)
HTSRLQP

SEQ ID NO: 118 - protein, 32 aa, synthetic construct
  REGION 1..32: note = LFR3 of the light chain (Lc) of the anti-IGFR-L1 antibody 6(AB-6)
GAPSRFSGSGSGKDYSLTISGLESEDIATYYC

SEQ ID NO: 119 - protein, 8 aa, synthetic construct
  REGION 1..8: note = CDR-L3 of the light chain (Lc) of the anti-IGFR-L1 antibody 6(AB-6)
QQTYTPYT

SEQ ID NO: 120 - protein, 13 aa, synthetic construct
  REGION 1..13: note = LFR4 of the light chain (Lc) of the anti-IGFR-L1 antibody 6(AB-6)
FGAGTKLELRRAD

SEQ ID NO: 121 - protein, 13 aa, synthetic construct
  REGION 1..13: note = Tail of the light chain (Lc) of the anti-IGFR-L1 antibody 6(AB-6)
AAPTVSISHHHEA

SEQ ID NO: 122 - protein, 230 aa, synthetic construct
  REGION 1..230: note = Heavy chain (Hc) of the anti-IGFR-L1 antibody 7 (AB-7)
  REGION 1..18: note = MISC_FEATURE - Leader sequence (L)
  REGION 19..48: note = MISC_FEATURE - Framework region HFR1
  REGION 49..53: note = MISC_FEATURE - Complementarity-determining region CDR-H1
  REGION 54..67: note = MISC_FEATURE - Framework region HFR2
  REGION 68..84: note = MISC_FEATURE - Complementarity-determining region CDR-H2
  REGION 85..116: note = MISC_FEATURE - Framework region HFR3
  REGION 117..128: note = MISC_FEATURE - Complementarity-determining region CDR-H3
  REGION 129..139: note = MISC_FEATURE - Framework region HFR4
  REGION 140..230: note = MISC_FEATURE - Tail region
MGWICIIFLVAIVTGAHSQVKLLQSGAALVKPGDSVKMSCKASGYSFTDYSIHWVKQSHGKSLEWIGYINPYSGGTTYNEKFKRKATLTVDKSSSTAYMEFSRLTSEDSAIYYCAREGMGITLYVMDAWGQGASVTVSSAETTAPSVYPLAPGTALKSNSMVTLGCLVKGYFPEPVTVTWNSGALSSGVHTFPAVLQSGLYTLTSSVTVPSSTWPSQTVTCNVAHPGPST

SEQ ID NO: 123 - protein, 18 aa, synthetic construct
  REGION 1..18: note = Leader (L) of the heavy chain (Hc) of the anti-IGFR-L1 antibody 7(AB-7)
MGWICIIFLVAIVTGAHS

SEQ ID NO: 124 - protein, 30 aa, synthetic construct
  REGION 1..30: note = HFR1 of the heavy chain (Hc) of the anti-IGFR-L1 antibody 7(AB-7)
QVKLLQSGAALVKPGDSVKMSCKASGYSFT

SEQ ID NO: 125 - protein, 5 aa, synthetic construct
  REGION 1..5: note = CDR-H1 of the heavy chain (Hc) of the anti-IGFR-L1 antibody 7(AB-7)
DYSIH

SEQ ID NO: 126 - protein, 14 aa, synthetic construct
  REGION 1..14: note = HFR2 of the heavy chain (Hc) of the anti-IGFR-L1 antibody 7(AB-7)
WVKQSHGKSLEWIG

SEQ ID NO: 127 - protein, 17 aa, synthetic construct
  REGION 1..17: note = CDR-H2 of the heavy chain (Hc) of the anti-IGFR-L1 antibody 7(AB-7)
YINPYSGGTTYNEKFKR

SEQ ID NO: 128 - protein, 32 aa, synthetic construct
  REGION 1..32: note = HFR3 of the heavy chain (Hc) of the anti-IGFR-L1 antibody 7(AB-7)
KATLTVDKSSSTAYMEFSRLTSEDSAIYYCAR

SEQ ID NO: 129 - protein, 12 aa, synthetic construct
  REGION 1..12: note = CDR-H3 of the heavy chain (Hc) of the anti-IGFR-L1 antibody 7(AB-7)
EGMGITLYVMDA

SEQ ID NO: 130 - protein, 11 aa, synthetic construct
  REGION 1..11: note = HFR4 of the heavy chain (Hc) of the anti-IGFR-L1 antibody 7(AB-7)
WGQGASVTVSS

SEQ ID NO: 131 - protein, 91 aa, synthetic construct
  REGION 1..91: note = Tail of the heavy chain (Hc) of the anti-IGFR-L1 antibody 7(AB-7)
AETTAPSVYPLAPGTALKSNSMVTLGCLVKGYFPEPVTVTWNSGALSSGVHTFPAVLQSGLYTLTSSVTVPSSTWPSQTVTCNVAHPGPST

SEQ ID NO: 132 - protein, 250 aa, synthetic construct
  REGION 1..250: note = Light chain (Lc) of the anti-IGFR-L1 antibody 7 (AB-7)
  REGION 1..44: note = MISC_FEATURE - Leader sequence (L)
  REGION 45..66: note = MISC_FEATURE - Framework region LFR1
  REGION 67..81: note = MISC_FEATURE - Complementarity-determining region CDR-L1
  REGION 82..96: note = MISC_FEATURE - Framework region LFR2
  REGION 97..103: note = MISC_FEATURE - Complementarity-determining region CDR-L2
  REGION 104..135: note = MISC_FEATURE - Framework region LFR3
  REGION 136..144: note = MISC_FEATURE - Complementarity-determining region CDR-L3
  REGION 145..156: note = MISC_FEATURE - Framework region LFR4
  REGION 157..250: note = MISC_FEATURE - Tail region
FFLGPPGGGGGGGGGEHPLFQHSEMETDRLLLWALLLWVPGSTGDIVLTQSPALAVSLGQRATVSCKASQTVTISRYHLLHWYQQKPGQQPTLLIYRASNLASGIPARFSGSGSGTDFTLTINPVQADDLASYYCQQSWGSPWTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSS

SEQ ID NO: 133 - protein, 44 aa, synthetic construct
  REGION 1..44: note = Leader (L) of the light chain (Lc) of the anti-IGFR-L1 antibody 7(AB-7)
FFLGPPGGGGGGGGGEHPLFQHSEMETDRLLLWALLLWVPGSTG

SEQ ID NO: 134 - protein, 22 aa, synthetic construct
  REGION 1..22: note = LFR1 of the light chain (Lc) of the anti-IGFR-L1 antibody 7(AB-7)
DIVLTQSPALAVSLGQRATVSC

SEQ ID NO: 135 - protein, 15 aa, synthetic construct
  REGION 1..15: note = CDR-L1 of the light chain (Lc) of the anti-IGFR-L1 antibody 7(AB-7)
KASQTVTISRYHLLH

SEQ ID NO: 136 - protein, 15 aa, synthetic construct
  REGION 1..15: note = LFR2 of the light chain (Lc) of the anti-IGFR-L1 antibody 7(AB-7)
WYQQKPGQQPTLLIY

SEQ ID NO: 137 - protein, 7 aa, synthetic construct
  REGION 1..7: note = CDR-L2 of the light chain (Lc) of the anti-IGFR-L1 antibody 7(AB-7)
RASNLAS

SEQ ID NO: 138 - protein, 32 aa, synthetic construct
  REGION 1..32: note = LFR3 of the light chain (Lc) of the anti-IGFR-L1 antibody 7(AB-7)
GIPARFSGSGSGTDFTLTINPVQADDLASYYC

SEQ ID NO: 139 - protein, 9 aa, synthetic construct
  REGION 1..9: note = CDR-L3 of the light chain (Lc) of the anti-IGFR-L1 antibody 7(AB-7)
QQSWGSPWT

SEQ ID NO: 140 - protein, 12 aa, synthetic construct
  REGION 1..12: note = LFR4 of the light chain (Lc) of the anti-IGFR-L1 antibody 7(AB-7)
FGGGTKLELKRA

SEQ ID NO: 141 - protein, 94 aa, synthetic construct
  REGION 1..94: note = Tail of the light chain (Lc) of the anti-IGFR-L1 antibody 7(AB-7)
DAAPTVSIFPPSTEQLATGGASVVCLMNNFYPRDISVKWKIDGTERRDGVLDSVTDQDSKDSTYSMSSTLSLTKADYESHNLYTCEVVHKTSSS

SEQ ID NO: 142 - protein, 227 aa, synthetic construct
  REGION 1..227: note = Heavy chain (Hc) of the anti-IGFR-L1 antibody 8 (AB-8)
  REGION 1..19: note = MISC_FEATURE - Leader sequence (L)
  REGION 20..49: note = MISC_FEATURE - Framework region HFR1
  REGION 50..54: note = MISC_FEATURE - Complementarity-determining region CDR-H1
  REGION 55..68: note = MISC_FEATURE - Framework region HFR2
  REGION 69..85: note = MISC_FEATURE - Complementarity-determining region CDR-H2
  REGION 86..117: note = MISC_FEATURE - Framework region HFR3
  REGION 118..128: note = MISC_FEATURE - Complementarity-determining region CDR-H3
  REGION 129..139: note = MISC_FEATURE - Framework region HFR4
  REGION 140..227: note = MISC_FEATURE - Tail region
MEWNWVFLFLLSVTAEVHSQVQLQQSGAELAKPGSSVKISCKASGYTFTSYFISWIKQTTGQDLEYIGYINTGSGGTNYNEKFKGKATLTVDRSSSTAFMQLSSLTPDDSAVYYCARELRRVRGWFAYWGQGTLVTVSSAETTAPSVYPLAPGTALKSNSMVTLGCLVKGYFPEPVTVTWNSGALSSGVHTFPAVLQSGLYTLTSSVTVPSSTWPSQTVTCNEPPAN

SEQ ID NO: 143 - protein, 19 aa, synthetic construct
  REGION 1..19: note = Leader (L) of the heavy chain (Hc) of the anti-IGFR-L1 antibody 8(AB-8)
MEWNWVFLFLLSVTAEVHS

SEQ ID NO: 144 - protein, 30 aa, synthetic construct
  REGION 1..30: note = HFR1 of the heavy chain (Hc) of the anti-IGFR-L1 antibody 8(AB-8)
QVQLQQSGAELAKPGSSVKISCKASGYTFT

SEQ ID NO: 145 - protein, 5 aa, synthetic construct
  REGION 1..5: note = CDR-H1 of the heavy chain (Hc) of the anti-IGFR-L1 antibody 8(AB-8)
SYFIS

SEQ ID NO: 146 - protein, 14 aa, synthetic construct
  REGION 1..14: note = HFR2 of the heavy chain (Hc) of the anti-IGFR-L1 antibody 8(AB-8)
WIKQTTGQDLEYIG

SEQ ID NO: 147 - protein, 17 aa, synthetic construct
  REGION 1..17: note = CDR-H2 of the heavy chain (Hc) of the anti-IGFR-L1 antibody 8(AB-8)
YINTGSGGTNYNEKFKG

SEQ ID NO: 148 - protein, 32 aa, synthetic construct
  REGION 1..32: note = HFR3 of the heavy chain (Hc) of the anti-IGFR-L1 antibody 8(AB-8)
KATLTVDRSSSTAFMQLSSLTPDDSAVYYCAR

SEQ ID NO: 149 - protein, 11 aa, synthetic construct
  REGION 1..11: note = CDR-H3 of the heavy chain (Hc) of the anti-IGFR-L1 antibody 8(AB-8)
ELRRVRGWFAY

SEQ ID NO: 150 - protein, 11 aa, synthetic construct
  REGION 1..11: note = HFR4 of the heavy chain (Hc) of the anti-IGFR-L1 antibody 8(AB-8)
WGQGTLVTVSS

SEQ ID NO: 151 - protein, 88 aa, synthetic construct
  REGION 1..88: note = Tail of the heavy chain (Hc) of the anti-IGFR-L1 antibody 8(AB-8)
AETTAPSVYPLAPGTALKSNSMVTLGCLVKGYFPEPVTVTWNSGALSSGVHTFPAVLQSGLYTLTSSVTVPSSTWPSQTVTCNEPPAN

SEQ ID NO: 152 - protein, 241 aa, synthetic construct
  REGION 1..241: note = Light chain (Lc) of the anti-IGFR-L1 antibody 8 (AB-8)
  REGION 1..39: note = MISC_FEATURE - Leader sequence (L)
  REGION 40..62: note = MISC_FEATURE - Framework region LFR1
  REGION 63..73: note = MISC_FEATURE - Complementarity-determining region CDR-L1
  REGION 74..88: note = MISC_FEATURE - Framework region LFR2
  REGION 89..95: note = MISC_FEATURE - Complementarity-determining region CDR-L2
  REGION 96..127: note = MISC_FEATURE - Framework region LFR3
  REGION 128..136: note = MISC_FEATURE - Complementarity-determining region CDR-L3
  REGION 137..148: note = MISC_FEATURE - Framework region LFR4
  REGION 149..241: note = MISC_FEATURE - Tail region
SLSGGGGGGGSASHRSHTDMGVPTQLLGLLLLWITDAICDTQMTQSPASLFASLGETVTIQCQASEDISSGLAWYQQKPGKSPHLLIYGANSLQDGVPSRFSGSGSGTQYSLKITNMQADDEGVYFCQQGLKYPFTFGSGTKLEIKRADAAPTVSIFPPSTEQLATGGASVVCLMNNFYPRDISVKWKIDGTERRDGVLDSVTDQDSKDSTYSMSSTLSLTKADYESHNLYTCEVFIRLSI

SEQ ID NO: 153 - protein, 39 aa, synthetic construct
  REGION 1..39: note = Leader (L) of the light chain (Lc) of the anti-IGFR-L1 antibody 8(AB-8)
SLSGGGGGGGSASHRSHTDMGVPTQLLGLLLLWITDAIC

SEQ ID NO: 154 - protein, 23 aa, synthetic construct
  REGION 1..23: note = LFR1 of the light chain (Lc) of the anti-IGFR-L1 antibody 8(AB-8)
DTQMTQSPASLFASLGETVTIQC

SEQ ID NO: 155 - protein, 11 aa, synthetic construct
  REGION 1..11: note = CDR-L1 of the light chain (Lc) of the anti-IGFR-L1 antibody 8(AB-8)
QASEDISSGLA

SEQ ID NO: 156 - protein, 15 aa, synthetic construct
  REGION 1..15: note = LFR2 of the light chain (Lc) of the anti-IGFR-L1 antibody 8(AB-8)
WYQQKPGKSPHLLIY

SEQ ID NO: 157 - protein, 7 aa, synthetic construct
  REGION 1..7: note = CDR-L2 of the light chain (Lc) of the anti-IGFR-L1 antibody 8(AB-8)
GANSLQD

SEQ ID NO: 158 - protein, 32 aa, synthetic construct
  REGION 1..32: note = LFR3 of the light chain (Lc) of the anti-IGFR-L1 antibody 8(AB-8)
GVPSRFSGSGSGTQYSLKITNMQADDEGVYFC

SEQ ID NO: 159 - protein, 9 aa, synthetic construct
  REGION 1..9: note = CDR-L3 of the light chain (Lc) of the anti-IGFR-L1 antibody 8(AB-8)
QQGLKYPFT

SEQ ID NO: 160 - protein, 12 aa, synthetic construct
  REGION 1..12: note = LFR4 of the light chain (Lc) of the anti-IGFR-L1 antibody 8(AB-8)
FGSGTKLEIKRA

SEQ ID NO: 161 - protein, 93 aa, synthetic construct
  REGION 1..93: note = Tail of the light chain (Lc) of the anti-IGFR-L1 antibody 8(AB-8)
DAAPTVSIFPPSTEQLATGGASVVCLMNNFYPRDISVKWKIDGTERRDGVLDSVTDQDSKDSTYSMSSTLSLTKADYESHNLYTCEVFIRLSI

SEQ ID NO: 162 - protein, 223 aa, synthetic construct
  REGION 1..223: note = Heavy chain (Hc) of the anti-IGFR-L1 antibody 9 (AB-9)
  REGION 1..19: note = MISC_FEATURE - Leader sequence (L)
  REGION 20..49: note = MISC_FEATURE - Framework region HFR1
  REGION 50..54: note = MISC_FEATURE - Complementarity-determining region CDR-H1
  REGION 55..68: note = MISC_FEATURE - Framework region HFR2
  REGION 69..85: note = MISC_FEATURE - Complementarity-determining region CDR-H2
  REGION 86..117: note = MISC_FEATURE - Framework region HFR3
  REGION 118..122: note = MISC_FEATURE - Complementarity-determining region CDR-H3
  REGION 123..133: note = MISC_FEATURE - Framework region HFR4
  REGION 134..223: note = MISC_FEATURE - Tail region
MDIRLSLAFLVLFIKGVQCEVQLVESGGGLVQPTGSVKLSCAASGFIFTNYGMAWVRQAPKKGLEWVAYISYDGGSTYFRDSVRGRFTISRDNAKSTLYLHMDSLRSEDTATYYCTTPRLDTWGQGVMVTVSSAETTAPSVYPLAPGTALKSNSMVTLGCLVKGYFPEPVTVTWNSGALSSGVHTFPAVLQSGLYTLTSSVTVPSSTCPARPHLQRATGTLFF

SEQ ID NO: 163 - protein, 19 aa, synthetic construct
  REGION 1..19: note = Leader (L) of the heavy chain (Hc) of the anti-IGFR-L1 antibody 9(AB-9)
MDIRLSLAFLVLFIKGVQC

SEQ ID NO: 164 - protein, 30 aa, synthetic construct
  REGION 1..30: note = HFR1 of the heavy chain (Hc) of the anti-IGFR-L1 antibody 9(AB-9)
EVQLVESGGGLVQPTGSVKLSCAASGFIFT

SEQ ID NO: 165 - protein, 5 aa, synthetic construct
  REGION 1..5: note = CDR-H1 of the heavy chain (Hc) of the anti-IGFR-L1 antibody 9(AB-9)
NYGMA

SEQ ID NO: 166 - protein, 14 aa, synthetic construct
  REGION 1..14: note = HFR2 of the heavy chain (Hc) of the anti-IGFR-L1 antibody 9(AB-9)
WVRQAPKKGLEWVA

SEQ ID NO: 167 - protein, 17 aa, synthetic construct
  REGION 1..17: note = CDR-H2 of the heavy chain (Hc) of the anti-IGFR-L1 antibody 9(AB-9)
YISYDGGSTYFRDSVRG

SEQ ID NO: 168 - protein, 32 aa, synthetic construct
  REGION 1..32: note = HFR3 of the heavy chain (Hc) of the anti-IGFR-L1 antibody 9(AB-9)
RFTISRDNAKSTLYLHMDSLRSEDTATYYCTT

SEQ ID NO: 169 - protein, 5 aa, synthetic construct
  REGION 1..5: note = CDR-H3 of the heavy chain (Hc) of the anti-IGFR-L1 antibody 9(AB-9)
PRLDT

SEQ ID NO: 170 - protein, 11 aa, synthetic construct
  REGION 1..11: note = HFR4 of the heavy chain (Hc) of the anti-IGFR-L1 antibody 9(AB-9)
WGQGVMVTVSS

SEQ ID NO: 171 - protein, 90 aa, synthetic construct
  REGION 1..90: note = Tail of the heavy chain (Hc) of the anti-IGFR-L1 antibody 9(AB-9)
AETTAPSVYPLAPGTALKSNSMVTLGCLVKGYFPEPVTVTWNSGALSSGVHTFPAVLQSGLYTLTSSVTVPSSTCPARPHLQRATGTLFF

SEQ ID NO: 172 - protein, 242 aa, synthetic construct
  REGION 1..242: note = Light chain (Lc) of the anti-IGFR-L1 antibody 9 (AB-9)
  REGION 1..39: note = MISC_FEATURE - Leader sequence (L)
  REGION 40..62: note = MISC_FEATURE - Framework region LFR1
  REGION 63..73: note = MISC_FEATURE - Complementarity-determining region CDR-L1
  REGION 74..88: note = MISC_FEATURE - Framework region LFR2
  REGION 89..95: note = MISC_FEATURE - Complementarity-determining region CDR-L2
  REGION 96..127: note = MISC_FEATURE - Framework region LFR3
  REGION 128..136: note = MISC_FEATURE - Complementarity-determining region CDR-L3
  REGION 137..148: note = MISC_FEATURE - Framework region LFR4
  REGION 149..242: note = MISC_FEATURE - Tail region
PLVGGGGGGLSLQSELNMDMRAHTQFIGLLWLWFAGARCDIQMTQSPSSLSASLGDRVTITCRPSQDIGNHLSWFQQKPGKSPRLMTYGATILEDGVPSRFSGSRSRSDYSLTISSLESEDTAIYYCLQYDKYPRTFGGGTKVELKRADAAPTVSIFPPSTEQLATGGASVVCLMNNFYPRDISVKWKIDGTERRDGVLDSVTDQDSKDSTYSMSSTLSLTKADYESHNLYTCEVFIKNSLK

SEQ ID NO: 173 - protein, 39 aa, synthetic construct
  REGION 1..39: note = Leader (L) of the light chain (Lc) of the anti-IGFR-L1 antibody 9(AB-9)
PLVGGGGGGLSLQSELNMDMRAHTQFIGLLWLWFAGARC

SEQ ID NO: 174 - protein, 23 aa, synthetic construct
  REGION 1..23: note = LFR1 of the light chain (Lc) of the anti-IGFR-L1 antibody 9(AB-9)
DIQMTQSPSSLSASLGDRVTITC

SEQ ID NO: 175 - protein, 11 aa, synthetic construct
  REGION 1..11: note = CDR-L1 of the light chain (Lc) of the anti-IGFR-L1 antibody 9(AB-9)
RPSQDIGNHLS

SEQ ID NO: 176 - protein, 15 aa, synthetic construct
  REGION 1..15: note = LFR2 of the light chain (Lc) of the anti-IGFR-L1 antibody 9(AB-9)
WFQQKPGKSPRLMTY

SEQ ID NO: 177 - protein, 7 aa, synthetic construct
  REGION 1..7: note = CDR-L2 of the light chain (Lc) of the anti-IGFR-L1 antibody 9(AB-9)
GATILED

SEQ ID NO: 178 - protein, 32 aa, synthetic construct
  REGION 1..32: note = LFR3 of the light chain (Lc) of the anti-IGFR-L1 antibody 9(AB-9)
GVPSRFSGSRSRSDYSLTISSLESEDTAIYYC

SEQ ID NO: 179 - protein, 9 aa, synthetic construct
  REGION 1..9: note = CDR-L3 of the light chain (Lc) of the anti-IGFR-L1 antibody 9(AB-9)
LQYDKYPRT

SEQ ID NO: 180 - protein, 12 aa, synthetic construct
  REGION 1..12: note = LFR4 of the light chain (Lc) of the anti-IGFR-L1 antibody 9(AB-9)
FGGGTKVELKRA

SEQ ID NO: 181 - protein, 94 aa, synthetic construct
  REGION 1..94: note = Tail of the light chain (Lc) of the anti-IGFR-L1 antibody 9(AB-9)
DAAPTVSIFPPSTEQLATGGASVVCLMNNFYPRDISVKWKIDGTERRDGVLDSVTDQDSKDSTYSMSSTLSLTKADYESHNLYTCEVFIKNSLK

SEQ ID NO: 182 - protein, 263 aa, synthetic construct
  REGION 1..263: note = Heavy chain (Hc) of the anti-IGFR-L1 antibody 10 (AB-10)
  REGION 1..53: note = MISC_FEATURE - Leader sequence (L)
  REGION 54..83: note = MISC_FEATURE - Framework region HFR1
  REGION 84..88: note = MISC_FEATURE - Complementarity-determining region CDR-H1
  REGION 89..102: note = MISC_FEATURE - Framework region HFR2
  REGION 103..119: note = MISC_FEATURE - Complementarity-determining region CDR-H2
  REGION 120..151: note = MISC_FEATURE - Framework region HFR3
  REGION 152..164: note = MISC_FEATURE - Complementarity-determining region CDR-H3
  REGION 165..175: note = MISC_FEATURE - Framework region HFR4
  REGION 176..263: note = MISC_FEATURE - Tail region
IWGGGGGGDGGAQSWTLRFSHSVISTAALHRLLTMDIRLSLAFLVLFIKGVQCGVQLVESGGGLVQSGRSIKVSCEASGFTFSNFYMAWVRQAPTKGLEWVASISSGGGNTHYRDSVRGRFTISRNNAKSTLYLQMDSLNSEDTATYYCARDPGGGSPYWYLDFWGPGTMVTVSSAETTAPSVYPLAPGTALKSNSMVTLGCLVKGYFPEPVTVTWNSGALSSGVHTFPAVLQSGLYTLTSSVTVPSSTWSSQAVTCNEPPAD

SEQ ID NO: 183 - protein, 53 aa, synthetic construct
  REGION 1..53: note = Leader (L) of the heavy chain (Hc) of the anti-IGFR-L1 antibody10 (AB-10)
IWGGGGGGDGGAQSWTLRFSHSVISTAALHRLLTMDIRLSLAFLVLFIKGVQC

SEQ ID NO: 184 - protein, 30 aa, synthetic construct
  REGION 1..30: note = HFR1 of the heavy chain (Hc) of the anti-IGFR-L1 antibody 10(AB-10)
GVQLVESGGGLVQSGRSIKVSCEASGFTFS

SEQ ID NO: 185 - protein, 5 aa, synthetic construct
  REGION 1..5: note = CDR-H1 of the heavy chain (Hc) of the anti-IGFR-L1 antibody 10(AB-10)
NFYMA

SEQ ID NO: 186 - protein, 14 aa, synthetic construct
  REGION 1..14: note = HFR2 of the heavy chain (Hc) of the anti-IGFR-L1 antibody 10(AB-10)
WVRQAPTKGLEWVA

SEQ ID NO: 187 - protein, 17 aa, synthetic construct
  REGION 1..17: note = CDR-H2 of the heavy chain (Hc) of the anti-IGFR-L1 antibody 10(AB-10)
SISSGGGNTHYRDSVRG

SEQ ID NO: 188 - protein, 32 aa, synthetic construct
  REGION 1..32: note = HFR3 of the heavy chain (Hc) of the anti-IGFR-L1 antibody 10(AB-10)
RFTISRNNAKSTLYLQMDSLNSEDTATYYCAR

SEQ ID NO: 189 - protein, 13 aa, synthetic construct
  REGION 1..13: note = CDR-H3 of the heavy chain (Hc) of the anti-IGFR-L1 antibody 10(AB-10)
DPGGGSPYWYLDF

SEQ ID NO: 190 - protein, 11 aa, synthetic construct
  REGION 1..11: note = HFR4 of the heavy chain (Hc) of the anti-IGFR-L1 antibody 10(AB-10)
WGPGTMVTVSS

SEQ ID NO: 191 - protein, 88 aa, synthetic construct
  REGION 1..88: note = Tail of the heavy chain (Hc) of the anti-IGFR-L1 antibody 10(AB-10)
AETTAPSVYPLAPGTALKSNSMVTLGCLVKGYFPEPVTVTWNSGALSSGVHTFPAVLQSGLYTLTSSVTVPSSTWSSQAVTCNEPPAD

SEQ ID NO: 192 - protein, 238 aa, synthetic construct
  REGION 1..238: note = Light chain (Lc) of the anti-IGFR-L1 antibody 10 (AB-10)
  REGION 1..37: note = MISC_FEATURE - Leader sequence (L)
  REGION 38..59: note = MISC_FEATURE - Framework region LFR1
  REGION 60..74: note = MISC_FEATURE - Complementarity-determining region CDR-L1
  REGION 75..89: note = MISC_FEATURE - Framework region LFR2
  REGION 90..96: note = MISC_FEATURE - Complementarity-determining region CDR-L2
  REGION 97..128: note = MISC_FEATURE - Framework region LFR3
  REGION 129..137: note = MISC_FEATURE - Complementarity-determining region CDR-L3
  REGION 138..149: note = MISC_FEATURE - Framework region LFR4
  REGION 150..238: note = MISC_FEATURE - Tail region
FWGGGGGGGQPLFQLSEMKTDTLLLWALLLWVPGCTGDIVLTQSPALAVSLGQRATISCRANQNVDFYDNTYLHWYQQKPGQQPKLLIYLASNLASGIPARFSGGGSGTDFTLTIDPVEADDTATYYCQQSRNLPNTFGAGTKLELKRADAAPTVSIFPPSTEQLATGGASVVCLMNNFYPRDISVKWKIDGTERRDGVLDSVTDQDSKDSTYSMSSTLSLTKADYESHNLYTCEVVH

SEQ ID NO: 193 - protein, 37 aa, synthetic construct
  REGION 1..37: note = Leader (L) of the light chain (Lc) of the anti-IGFR-L1 antibody10 (AB-10)
FWGGGGGGGQPLFQLSEMKTDTLLLWALLLWVPGCTG

SEQ ID NO: 194 - protein, 22 aa, synthetic construct
  REGION 1..22: note = LFR1 of the light chain (Lc) of the anti-IGFR-L1 antibody 10(AB-10)
DIVLTQSPALAVSLGQRATISC

SEQ ID NO: 195 - protein, 15 aa, synthetic construct
  REGION 1..15: note = CDR-L1 of the light chain (Lc) of the anti-IGFR-L1 antibody 10(AB-10)
RANQNVDFYDNTYLH

SEQ ID NO: 196 - protein, 15 aa, synthetic construct
  REGION 1..15: note = LFR2 of the light chain (Lc) of the anti-IGFR-L1 antibody 10(AB-10)
WYQQKPGQQPKLLIY

SEQ ID NO: 197 - protein, 7 aa, synthetic construct
  REGION 1..7: note = CDR-L2 of the light chain (Lc) of the anti-IGFR-L1 antibody 10(AB-10)
LASNLAS

SEQ ID NO: 198 - protein, 32 aa, synthetic construct
  REGION 1..32: note = LFR3 of the light chain (Lc) of the anti-IGFR-L1 antibody 10(AB-10)
GIPARFSGGGSGTDFTLTIDPVEADDTATYYC

SEQ ID NO: 199 - protein, 9 aa, synthetic construct
  REGION 1..9: note = CDR-L3 of the light chain (Lc) of the anti-IGFR-L1 antibody 10(AB-10)
QQSRNLPNT

SEQ ID NO: 200 - protein, 12 aa, synthetic construct
  REGION 1..12: note = LFR4 of the light chain (Lc) of the anti-IGFR-L1 antibody 10(AB-10)
FGAGTKLELKRA

SEQ ID NO: 201 - protein, 89 aa, synthetic construct
  REGION 1..89: note = Tail of the light chain (Lc) of the anti-IGFR-L1 antibody 10(AB-10)
DAAPTVSIFPPSTEQLATGGASVVCLMNNFYPRDISVKWKIDGTERRDGVLDSVTDQDSKDSTYSMSSTLSLTKADYESHNLYTCEVVH

SEQ ID NO: 202 - protein, 256 aa, synthetic construct
  REGION 1..256: note = Heavy chain (Hc) of the anti-IGFR-L1 antibody 11 (AB-11)
  REGION 1..54: note = MISC_FEATURE - Leader sequence (L)
  REGION 55..84: note = MISC_FEATURE - Framework region HFR1
  REGION 85..89: note = MISC_FEATURE - Complementarity-determining region CDR-H1
  REGION 90..103: note = MISC_FEATURE - Framework region HFR2
  REGION 104..120: note = MISC_FEATURE - Complementarity-determining region CDR-H2
  REGION 121..152: note = MISC_FEATURE - Framework region HFR3
  REGION 153..159: note = MISC_FEATURE - Complementarity-determining region CDR-H3
  REGION 160..170: note = MISC_FEATURE - Framework region HFR4
  REGION 171..256: note = MISC_FEATURE - Tail region
SGGGGGGGWDGGAQSWTLRFSHSVISAAALHRLLTMDIRLSLVFLVPFIKGVQCEVQLVESDGGFVQPGRSLKLSCAASGFTFSDYYMSWVRQAPTKGLEWVATIGYDGTYTNYRDSVKGRFTISRDNARSILYLQMDSLRSEDTATYYCARHPSYFDHWGQGVMVTVSSAQTTAPSVYPLAPGCGDTTSSTVTLGCLVKGYFPEPVTVTWNSGALSSDVHTFPAVLQSGLYTLTSSVTSSTWPSQTVTCNEPPAK

SEQ ID NO: 203 - protein, 54 aa, synthetic construct
  REGION 1..54: note = Leader (L) of the heavy chain (Hc) of the anti-IGFR-L1 antibody11 (AB-11)
SGGGGGGGWDGGAQSWTLRFSHSVISAAALHRLLTMDIRLSLVFLVPFIKGVQC

SEQ ID NO: 204 - protein, 30 aa, synthetic construct
  REGION 1..30: note = HFR1 of the heavy chain (Hc) of the anti-IGFR-L1 antibody 11(AB-11)
EVQLVESDGGFVQPGRSLKLSCAASGFTFS

SEQ ID NO: 205 - protein, 5 aa, synthetic construct
  REGION 1..5: note = CDR-H1 of the heavy chain (Hc) of the anti-IGFR-L1 antibody 11(AB-11)
DYYMS

SEQ ID NO: 206 - protein, 14 aa, synthetic construct
  REGION 1..14: note = HFR2 of the heavy chain (Hc) of the anti-IGFR-L1 antibody 11(AB-11)
WVRQAPTKGLEWVA

SEQ ID NO: 207 - protein, 17 aa, synthetic construct
  REGION 1..17: note = CDR-H2 of the heavy chain (Hc) of the anti-IGFR-L1 antibody 11(AB-11)
TIGYDGTYTNYRDSVKG

SEQ ID NO: 208 - protein, 32 aa, synthetic construct
  REGION 1..32: note = HFR3 of the heavy chain (Hc) of the anti-IGFR-L1 antibody 11(AB-11)
RFTISRDNARSILYLQMDSLRSEDTATYYCAR

SEQ ID NO: 209 - protein, 7 aa, synthetic construct
  REGION 1..7: note = CDR-H3 of the heavy chain (Hc) of the anti-IGFR-L1 antibody 11(AB-11)
HPSYFDH

SEQ ID NO: 210 - protein, 11 aa, synthetic construct
  REGION 1..11: note = HFR4 of the heavy chain (Hc) of the anti-IGFR-L1 antibody 11(AB-11)
WGQGVMVTVSS

SEQ ID NO: 211 - protein, 86 aa, synthetic construct
  REGION 1..86: note = Tail of the heavy chain (Hc) of the anti-IGFR-L1 antibody 11(AB-11)
AQTTAPSVYPLAPGCGDTTSSTVTLGCLVKGYFPEPVTVTWNSGALSSDVHTFPAVLQSGLYTLTSSVTSSTWPSQTVTCNEPPAK

SEQ ID NO: 212 - protein, 206 aa, synthetic construct
  REGION 1..206: note = Light chain (Lc) of the anti-IGFR-L1 antibody 11 (AB-11)
  REGION 1..20: note = MISC_FEATURE - Leader sequence (L)
  REGION 21..43: note = MISC_FEATURE - Framework region LFR1
  REGION 44..60: note = MISC_FEATURE - Complementarity-determining region CDR-L1
  REGION 61..75: note = MISC_FEATURE - Framework region LFR2
  REGION 76..82: note = MISC_FEATURE - Complementarity-determining region CDR-L2
  REGION 83..114: note = MISC_FEATURE - Framework region LFR3
  REGION 115..123: note = MISC_FEATURE - Complementarity-determining region CDR-L3
  REGION 124..135: note = MISC_FEATURE - Framework region LFR4
  REGION 136..206: note = MISC_FEATURE - Tail region
MESQTQVLMSLLLWISGTCGDIVMTQSPSSLAVSAGETVTINCKSSQSLLYSENQKNYLAWFQQRPGQSPKLLINWASTRQSGVPDRFIGSGSGTDFTLTISSVQAEDLATYHCQQYYDTPRTFGGGSKLELKRADAAPTVSIFPPSTEQLATGGASVVCLMNNFYPRDISVKWKIDGTERRDGVLDSVTESGQQRQLLYSMSSNI

SEQ ID NO: 213 - protein, 20 aa, synthetic construct
  REGION 1..20: note = Leader (L) of the light chain (Lc) of the anti-IGFR-L1 antibody11 (AB-11)
MESQTQVLMSLLLWISGTCG

SEQ ID NO: 214 - protein, 23 aa, synthetic construct
  REGION 1..23: note = LFR1 of the light chain (Lc) of the anti-IGFR-L1 antibody 11(AB-11)
DIVMTQSPSSLAVSAGETVTINC

SEQ ID NO: 215 - protein, 17 aa, synthetic construct
  REGION 1..17: note = CDR-L1 of the light chain (Lc) of the anti-IGFR-L1 antibody 11(AB-11)
KSSQSLLYSENQKNYLA

SEQ ID NO: 216 - protein, 15 aa, synthetic construct
  REGION 1..15: note = LFR2 of the light chain (Lc) of the anti-IGFR-L1 antibody 11(AB-11)
WFQQRPGQSPKLLIN

SEQ ID NO: 217 - protein, 7 aa, synthetic construct
  REGION 1..7: note = CDR-L2 of the light chain (Lc) of the anti-IGFR-L1 antibody 11(AB-11)
WASTRQS

SEQ ID NO: 218 - protein, 32 aa, synthetic construct
  REGION 1..32: note = LFR3 of the light chain (Lc) of the anti-IGFR-L1 antibody 11(AB-11)
GVPDRFIGSGSGTDFTLTISSVQAEDLATYHC

SEQ ID NO: 219 - protein, 9 aa, synthetic construct
  REGION 1..9: note = CDR-L3 of the light chain (Lc) of the anti-IGFR-L1 antibody 11(AB-11)
QQYYDTPRT

SEQ ID NO: 220 - protein, 12 aa, synthetic construct
  REGION 1..12: note = LFR4 of the light chain (Lc) of the anti-IGFR-L1 antibody 11(AB-11)
FGGGSKLELKRA

SEQ ID NO: 221 - protein, 71 aa, synthetic construct
  REGION 1..71: note = Tail of the light chain (Lc) of the anti-IGFR-L1 antibody 11(AB-11)
DAAPTVSIFPPSTEQLATGGASVVCLMNNFYPRDISVKWKIDGTERRDGVLDSVTESGQQRQLLYSMSSNI

SEQ ID NO: 222 - protein, 473 aa, synthetic construct
  REGION 1..473: note = Heavy chain (Hc) of the anti-IGFR-L1 antibody 12 (AB-12)
  REGION 1..19: note = MISC_FEATURE - Leader sequence (L)
  REGION 20..49: note = MISC_FEATURE - Framework region HFR1
  REGION 50..54: note = MISC_FEATURE - Complementarity-determining region CDR-H1
  REGION 55..68: note = MISC_FEATURE - Framework region HFR2
  REGION 69..87: note = MISC_FEATURE - Complementarity-determining region CDR-H2
  REGION 88..119: note = MISC_FEATURE - Framework region HFR3
  REGION 120..133: note = MISC_FEATURE - Complementarity-determining region CDR-H3
  REGION 134..144: note = MISC_FEATURE - Framework region HFR4
  REGION 145..473: note = MISC_FEATURE - Tail region
MKLWLNWIFLLTLLNGIQCEVKLLESGGGLVQPGGSMRLSCAASGFTFTDFYMNWIRQPAGKAPEWLGFIRNKANGYTTEYTPSVKGRFTISRDDTQNMLYLQMNTLRAEDTATYYCARHYYDGSYYYWYFDFWGPGTMVTVSSARTTAPSVYPLVPGCSGTSGSLVTLGCLVKGYFPEPVTVKWNSGALSSGVHTFPAVLQSGLYTLSSSVTVPSSTWSSQTVTCSVAHPATKSNLIKRIEPRRPKPRPPTDICSCDDNLGRPSVFIFPPKPKDILMITLTPKVTCVVVDVSEEEPDVQFSWFVDNVRVFTAQTQPHEEQLNGTFRVVSTLHIQHQDWMSGKEFKCKVNNKDLPSPIEKTISKPRGKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK

SEQ ID NO: 223 - protein, 19 aa, synthetic construct
  REGION 1..19: note = Leader (L) of the heavy chain (Hc) of the anti-IGFR-L1 antibody12 (AB-12)
MKLWLNWIFLLTLLNGIQC

SEQ ID NO: 224 - protein, 30 aa, synthetic construct
  REGION 1..30: note = HFR1 of the heavy chain (Hc) of the anti-IGFR-L1 antibody 12(AB-12)
EVKLLESGGGLVQPGGSMRLSCAASGFTFT

SEQ ID NO: 225 - protein, 5 aa, synthetic construct
  REGION 1..5: note = CDR-H1 of the heavy chain (Hc) of the anti-IGFR-L1 antibody 12(AB-12)
DFYMN

SEQ ID NO: 226 - protein, 14 aa, synthetic construct
  REGION 1..14: note = HFR2 of the heavy chain (Hc) of the anti-IGFR-L1 antibody 12(AB-12)
WIRQPAGKAPEWLG

SEQ ID NO: 227 - protein, 19 aa, synthetic construct
  REGION 1..19: note = CDR-H2 of the heavy chain (Hc) of the anti-IGFR-L1 antibody 12(AB-12)
FIRNKANGYTTEYTPSVKG

SEQ ID NO: 228 - protein, 32 aa, synthetic construct
  REGION 1..32: note = HFR3 of the heavy chain (Hc) of the anti-IGFR-L1 antibody 12(AB-12)
RFTISRDDTQNMLYLQMNTLRAEDTATYYCAR

SEQ ID NO: 229 - protein, 14 aa, synthetic construct
  REGION 1..14: note = CDR-H3 of the heavy chain (Hc) of the anti-IGFR-L1 antibody 12(AB-12)
HYYDGSYYYWYFDF

SEQ ID NO: 230 - protein, 11 aa, synthetic construct
  REGION 1..11: note = HFR4 of the heavy chain (Hc) of the anti-IGFR-L1 antibody 12(AB-12)
WGPGTMVTVSS

SEQ ID NO: 231 - protein, 329 aa, synthetic construct; identical to SEQ ID NO: 11
  REGION 1..329: note = Tail of the heavy chain (Hc) of the anti-IGFR-L1 antibody 12(AB-12)

SEQ ID NO: 232 - protein, 264 aa, synthetic construct
  REGION 1..264: note = Light chain (Lc) of the anti-IGFR-L1 antibody 12 (AB-12)
  REGION 1..51: note = MISC_FEATURE - Leader sequence (L)
  REGION 52..74: note = MISC_FEATURE - Framework region LFR1
  REGION 75..90: note = MISC_FEATURE - Complementarity-determining region CDR-L1
  REGION 91..105: note = MISC_FEATURE - Framework region LFR2
  REGION 106..112: note = MISC_FEATURE - Complementarity-determining region CDR-L2
  REGION 113..144: note = MISC_FEATURE - Framework region LFR3
  REGION 145..153: note = MISC_FEATURE - Complementarity-determining region CDR-L3
  REGION 154..165: note = MISC_FEATURE - Framework region LFR4
  REGION 166..264: note = MISC_FEATURE - Tail region
FFFFFSSPWGGGGGGGGGDQSSQSVSSSSFFKMKVPVRLLVLLFWIPASRSDVVLTQTPVAQPVTLGDQVSISCRSSQSLVKSNGNTYLEWFLQKPGQSPQLLIYKVSNRFSGVPDRFIGSGSGSDFTLKISRVEPEDLGVYYCFQAAHDPRTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPSNNP

SEQ ID NO: 233 - protein, 51 aa, synthetic construct
  REGION 1..51: note = Leader (L) of the light chain (Lc) of the anti-IGFR-L1 antibody12 (AB-12)
FFFFFSSPWGGGGGGGGGDQSSQSVSSSSFFKMKVPVRLLVLLFWIPASRS

SEQ ID NO: 234 - protein, 23 aa, synthetic construct
  REGION 1..23: note = LFR1 of the light chain (Lc) of the anti-IGFR-L1 antibody 12(AB-12)
DVVLTQTPVAQPVTLGDQVSISC

SEQ ID NO: 235 - protein, 16 aa, synthetic construct
  REGION 1..16: note = CDR-L1 of the light chain (Lc) of the anti-IGFR-L1 antibody 12(AB-12)
RSSQSLVKSNGNTYLE

SEQ ID NO: 236 - protein, 15 aa, synthetic construct
  REGION 1..15: note = LFR2 of the light chain (Lc) of the anti-IGFR-L1 antibody 12(AB-12)
WFLQKPGQSPQLLIY

SEQ ID NO: 237 - protein, 7 aa, synthetic construct
  REGION 1..7: note = CDR-L2 of the light chain (Lc) of the anti-IGFR-L1 antibody 12(AB-12)
KVSNRFS

SEQ ID NO: 238 - protein, 32 aa, synthetic construct
  REGION 1..32: note = LFR3 of the light chain (Lc) of the anti-IGFR-L1 antibody 12(AB-12)
GVPDRFIGSGSGSDFTLKISRVEPEDLGVYYC

SEQ ID NO: 239 - protein, 9 aa, synthetic construct
  REGION 1..9: note = CDR-L3 of the light chain (Lc) of the anti-IGFR-L1 antibody 12(AB-12)
FQAAHDPRT

SEQ ID NO: 240 - protein, 12 aa, synthetic construct
  REGION 1..12: note = LFR4 of the light chain (Lc) of the anti-IGFR-L1 antibody 12(AB-12)
FGGGTKLELKRA

SEQ ID NO: 241 - protein, 99 aa, synthetic construct
  REGION 1..99: note = Tail of the light chain (Lc) of the anti-IGFR-L1 antibody 12(AB-12)
DAAPTVSIFPPSTEQLATGGASVVCLMNNFYPRDISVKWKIDGTERRDGVLDSVTDQDSKDSTYSMSSTLSLTKADYESHNLYTCEVVHKTSSSPSNNP

SEQ ID NO: 242 - protein, 11 aa, Homo sapiens
CHQCDPDKYSE

SEQ ID NO: 243 - protein, 16 aa, Homo sapiens
MYKWAKPKICSEDLEG

SEQ ID NO: 244 - protein, 14 aa, Homo sapiens
CTFSRNTPTRTFNY

(19) United States
(12) Patent Application Publication
(10) Pub. No.: US 2024/0343789 A1
(43) Pub. Date: Oct. 17, 2024

(54) IGFR-L1 ANTIBODIES AND USES THEREOF

(71) Applicant: Helmholtz Zentrum München - Deutsches Forschungszentrum für Gesundheit und Umwelt (GmbH), Neuherberg (DE)

(72) Inventors: Heiko LICKERT, Muenchen (DE); Ünal COSKUN, Dresden (DE); Michal GRZYBEK, Bannewitz (DE)

(21) Appl. No.: 18/291,198

(22) PCT Filed: Jul. 25, 2022
(86) PCT No.: PCT/EP2022/070748

§ 371 Date: Jan. 22, 2024

(30) Foreign Application Priority Data
Jul. 23, 2021 (EP) 21187469.8
Jul. 26, 2021 (LU) LU500470

(51) Int. Cl. C07K 16/22; A61P 3/10; G01N 33/50

(52) U.S. Cl. CPC C07K 16/22; A61P 3/10; G01N 33/5041; C07K 2317/75; C07K 2317/92

(57) ABSTRACT

The present invention provides novel IGFR-L1 antibodies for targeting extracellular domain of non-denatured IGFR-L1 protein. Said antibodies are envisaged for use as a medicament, and in particular for treatment of diabetes and related disorders and cancer.

This application contains a Sequence Listing in computer readable form, which is incorporated herein by reference.

### TECHNICAL FIELD

The present invention provides novel structure-specific (e.g., conformation-specific) IGFR-L1 antibodies for specifically targeting extracellular domain of non-denatured IGFR-L1 protein and methods and uses based thereon (e.g., therapeutic and immunoprecipitation methods). The novel IGFR-L1 antibodies of the present invention are envisaged for use as a medicament and/or in therapy and in particular for treatment of diabetes and related disorders (e.g., Hyperinsulinism, insulin resistance and related disorders, etc.). The novel IGFR-L1 antibodies of the present invention are also envisaged for use in various immunoprecipitation assays, e.g., for which domain-specific peptide antibodies from the prior art are not suitable.

### BACKGROUND

The insulin/insulin-like growth factors (IGFs) constitute a network of ligands, cell-surface receptors, and binding proteins involved in the regulation of multiple physiological and pathological processes. Insulin/IGFs play key developmental and metabolic roles at every stage of life. Insulin/IGF signaling also contributes to regulation of lifespan, while dysregulation of signaling has been implicated in neoplasia. Although the insulin receptor (InsR) and IGF-1 receptor (IGF1R) share the majority of their downstream cytoplasmic mediators, most experimental and clinical evidence is consistent with the notion that InsR activation (mainly by insulin) leads primarily to metabolic activities, whereas IGF1R activation (mainly by IGF-1 or IGF-2) leads to proliferative and differentiation events (Sarfstein R and Werner H Endocrinology. 2013 May; 154(5):1672-9; Siddle K J Mol Endocrinol. 2011 Jun. 17; 47(1):R1-10).

InsR and IGF1R belong to a family of transmembrane tyrosine kinase-containing receptors. In their mature form, they present as heterotetramers composed of 2 extracellular α-subunits and 2 transmembrane β-subunits harboring the tyrosine kinase activity. Both IGF- and insulin receptors show a high degree of homology (84% in the tyrosine kinase domain, 45%-65% in the ligand binding domain, and above 50% in overall amino acid sequence). In addition, the receptors display a remarkable similarity in genomic organization (Sarfstein R and Werner H, loc. cit.; Arnalez F and Helman L. Hematol Oncol Clin North Am. 2012 June; 26(3):527-42).

There are also “hybrid” receptors composed of half an insulin receptor and half an IGF receptor (IRαβ linked to IGF1Rαβ). Hybrids bind IGFs with similar affinity to IGFR, but bind insulin with substantially lower affinity than InsR. It is unclear whether hybrid receptors have a distinct physiological role (Sarfstein R and Werner H, loc. cit.; Arnalez F and Helman L., loc. cit).

The insulin receptor exists in two splice variant isoforms as a result of alternative splicing of the sequence encoded by exon 11; the ‘B’ isoform recognizes only insulin, but the ‘A’ isoform, which is the isoform that is most commonly expressed by tumours, recognizes both insulin and IGF1 and 2. Both isoforms are differentially expressed during development, with InsR-A predominantly expressed in fetal tissues and InsR-B predominately expressed in adult tissues, particularly liver, muscle, and adipocytes. The IGF1R displays an opposite pattern of expression, being absent in liver and present at low levels in adipose tissue and at high levels in brain. In addition, and consistent with its potent anti-apoptotic, pro-survival role, the IGF1R is overexpressed in most tumors and malignant cells (Pollak M Nat Rev Cancer. 2012 Feb. 16; 12(3):159-69, Sarfstein R and Werner H, loc. cit.; Siddle K, loc cit).

IGF1 and IGF2 can be expressed in endocrine, paracrine or autocrine manners, the latter being common in transformed cells. The liver is their main site of production. By contrast, insulin production is confined to pancreatic β-cells. Insulin and the IGFs bind with high affinity to their specific receptor and with lower affinity to the non-cognate receptor, with the exception of IGF2, which also binds InsR-A with high affinity (Pollak M, loc cit.; Siddle K, loc. cit.).

Ligand binding induces conformational changes in the structures of the InsR and IGF1R and activates their intrinsic tyrosine kinase activity. Although insulin and IGFs play distinct physiological roles, they utilize the same signaling pathways. Downstream signaling of the InsR and IGF1R is mostly channeled through the MAPK/Ras-Raf-Erk pathway, the phosphatidylinositol-3-kinase/AKT/mTOR (PI3K/AKT) pathway and the Janus kinase/signal transducer and activator of transcription (JAK/STAT) pathway. Ultimately, activation of IGF1R results in increased cell proliferation and decreased apoptosis, whereas activation of the InsR by insulin-binding promotes the storage and synthesis of lipids, protein, and carbohydrates and inhibits their breakdown and release into the circulation. The first step by which insulin increases energy storage or utilization involves the regulated transport of glucose into the cell, mediated by the facilitative glucose transporter Glut4 (Chang et al. Mol Med. 2004 July-December; 10(7-12): 65-71). Insulin expression is confined to specialized pancreatic β-cells, and under normal circumstances it is tightly regulated by the level of circulating glucose. Insulin-stimulated glucose uptake by classic insulin-sensitive organs (liver, muscle and adipose tissue) reduces circulating glucose levels. The β-cells are thus glucose ‘thermostats’, sensing glucose and releasing insulin to maintain physiologic glucose levels within a relatively narrow range. Breakdown of the delicately balanced InsR signaling pathways results in an uncontrolled or impaired insulin-secretion, dysregulated blood glucose levels, and eventually to pancreatic β cell destruction or loss-of-function, a condition commonly known as diabetes (Pollak M, loc cit.; Siddle K, loc. cit.; Sarfstein R and Werner H, loc. cit; Arnalez F and Helman L., loc. cit)

Diabetes mellitus, affecting 8.3% of the adult population of the world and increasing at an alarming rate, is one of the most common diseases of current era. The number of diabetes mellitus patients is projected to increase from 382 million in 2013 to 592 million by 2035, denoting a net increase of 55%. The predominant form is type 2 diabetes (T2D) which accounts for nearly 90% of all diabetes cases (Hameed et al. World J Diabetes. 2015 May 15; 6(4): 598-612).

Type 1 diabetes (T1D) is an autoimmune disorder afflicting millions of people worldwide and occurs as a consequence of the organ-specific immune destruction of the insulin-producing β-cells in the islets of Langerhans within the pancreas. Once those cells are destroyed, patients with type 1 diabetes lose blood glucose control, which can result in both acute conditions (for example, ketoacidosis and severe hypoglycaemia) and secondary complications (including heart disease, blindness and kidney failure). Type 1 diabetes is thought to develop as a consequence of a combination of genetic predisposition, largely unknown environmental factors, and stochastic events, however, the precise immunologic, genetic and physiologic events that control disease initiation and progression continue to be elucidated.

Early type 2 diabetes (T2D) is caused by insulin resistance of classic insulin-target organs (i.e. reduced uptake of glucose by normal insulin-target cells, often induced by excess calorific intake) leading to hyperinsulinaemia. Initially, these increased levels of insulin are sufficient to overcome insulin resistance and to avoid hyperglycaemia. However, hyperglycaemia eventually occurs not only because of increasing insulin resistance but also because of decreasing insulin output by pancreatic β-cells.

Controlling blood glucose levels is the major goal of diabetes treatment. T1D is commonly managed with administration of insulin as well as dietary changes and exercise. However, the life-long requirement of insulin injections after nutritional intake may severely reduce quality of life of the patient. Moreover, appropriate dosage and timing of insulin injection can prove difficult. Cure or prevention of T1D is severely impaired by the absence of biomarkers that are reliably correlated with the pathogenic process, resulting in β-cell numbers being markedly reduced at the time of diagnosis. The goal of most clinical trials in type 1 diabetes today is to improve functional residual β-cell mass, optimally through induction of immunologic tolerance, while preserving protective immune responses. By definition, this will rarely “cure” the disease because of the significant β-cell destruction that preceded the treatment. Therefore, reliable biomarkers preferably expressed at disease onset would be highly desirable. Other methods focus on the transplantation of either the pancreas or pancreatic β-cells to reconstitute the insulin-secreting function. However, this technique is hampered by a shortage of donor organs. For T2D, besides insulin, other non-insulin therapeutics including synthetic blood sugar lowering agents are available, which are however often limited in terms of their practical effect, convenience of administration, and may elicit adverse reactions.

The safety concerns and adverse effects of available diabetes therapeutics, as well as the lack of permanent remission of disease with any agent tested to date have heightened interest in specific interventions that might modulate the disease.

It is the object of the invention to comply with the needs in the prior art.

### SUMMARY

The present invention relates to an antibody which binds to the extracellular domain of non-denatured (e.g., folded or partially folded) Inceptor protein comprising the amino acid sequence of SEQ ID NO: 1 (IGFR-L1 protein), preferably to a native epitope within IGFR-L1 protein, wherein binding to non-denatured IGFR-L1 protein by the antibody is determinable (e.g., determined) as follows:
(a) incubating an aliquot of a lysate from Min6 cells (ATCC CRL-11506) separately with each of
   (i) a buffer suitable for SDS-PAGE of proteins for 5 min at ambient temperature,
   (ii) a buffer suitable for SDS-PAGE of proteins and a reducing agent for 5 min at ambient temperature, and
   (iii) a buffer suitable for SDS-PAGE of proteins and a reducing agent for 5 min at 95° C.,
(b) subjecting said aliquots to SDS-PAGE;
(c) western blotting the SDS-PAGE gel;
(d) incubating the western blot membrane with the antibody;
wherein the antibody detects only aliquot (i).

The present invention further relates to an antibody, wherein the antibody is
(a) an antibody comprising a heavy chain variable region comprising heavy chain CDR1 having the amino acid sequence as set forth in SEQ ID NO: 5, heavy chain CDR2 having the amino acid sequence as set forth in SEQ ID NO: 7, and heavy chain CDR3 having the amino acid sequence as set forth in SEQ ID NO: 9 and a light chain variable region comprising light chain CDR1 having the amino acid sequence as set forth in SEQ ID NO: 15, light chain CDR2 having the amino acid sequence as set forth in SEQ ID NO: 17, and light chain CDR3 having the amino acid sequence as set forth in SEQ ID NO: 19;
(b) an antibody comprising a heavy chain variable region comprising heavy chain CDR1 having the amino acid sequence as set forth in SEQ ID NO: 25, heavy chain CDR2 having the amino acid sequence as set forth in SEQ ID NO: 27, and heavy chain CDR3 having the amino acid sequence as set forth in SEQ ID NO: 29, and a light chain variable region comprising light chain CDR1 having the amino acid sequence as set forth in SEQ ID NO: 35, light chain CDR2 having the amino acid sequence as set forth in SEQ ID NO: 37, and light chain CDR3 having the amino acid sequence as set forth in SEQ ID NO: 39;
(c) an antibody comprising a heavy chain variable region comprising heavy chain CDR1 having the amino acid sequence as set forth in SEQ ID NO: 45, heavy chain CDR2 having the amino acid sequence as set forth in SEQ ID NO: 47, and heavy chain CDR3 having the amino acid sequence as set forth in SEQ ID NO: 49, and a light chain variable region comprising light chain CDR1 having the amino acid sequence as set forth in SEQ ID NO: 55, light chain CDR2 having the amino acid sequence as set forth in SEQ ID NO: 57, and light chain CDR3 having the amino acid sequence as set forth in SEQ ID NO: 59;
(d) an antibody comprising a heavy chain variable region comprising heavy chain CDR1 having the amino acid sequence as set forth in SEQ ID NO: 65, heavy chain CDR2 having the amino acid sequence as set forth in SEQ ID NO: 67, and heavy chain CDR3 having the amino acid sequence as set forth in SEQ ID NO: 69, and a light chain variable region comprising light chain CDR1 having the amino acid sequence as set forth in SEQ ID NO: 75, light chain CDR2 having the amino acid sequence as set forth in SEQ ID NO: 77, and light chain CDR3 having the amino acid sequence as set forth in SEQ ID NO: 79;
(e) an antibody comprising a heavy chain variable region comprising heavy chain CDR1 having the amino acid sequence as set forth in SEQ ID NO: 85, heavy chain CDR2 having the amino acid sequence as set forth in SEQ ID NO: 87, and heavy chain CDR3 having the amino acid sequence as set forth in SEQ ID NO: 89, and a light chain variable region comprising light chain CDR1 having the amino acid sequence as set forth in SEQ ID NO: 95, light chain CDR2 having the amino acid sequence as set forth in SEQ ID NO: 97, and light chain CDR3 having the amino acid sequence as set forth in SEQ ID NO: 99;
(f) an antibody comprising a heavy chain variable region comprising heavy chain CDR1 having the amino acid sequence as set forth in SEQ ID NO: 105, heavy chain CDR2 having the amino acid sequence as set forth in SEQ ID NO: 107, and heavy chain CDR3 having the amino acid sequence as set forth in SEQ ID NO: 109, and a light chain variable region comprising light chain CDR1 having the amino acid sequence as set forth in SEQ ID NO: 115, light chain CDR2 having the amino acid sequence as set forth in SEQ ID NO: 117, and light chain CDR3 having the amino acid sequence as set forth in SEQ ID NO: 119;
(g) an antibody comprising a heavy chain variable region comprising heavy chain CDR1 having the amino acid sequence as set forth in SEQ ID NO: 125, heavy chain CDR2 having the amino acid sequence as set forth in SEQ ID NO: 127, and heavy chain CDR3 having the amino acid sequence as set forth in SEQ ID NO: 129, and a light chain variable region comprising light chain CDR1 having the amino acid sequence as set forth in SEQ ID NO: 135, light chain CDR2 having the amino acid sequence as set forth in SEQ ID NO: 137, and light chain CDR3 having the amino acid sequence as set forth in SEQ ID NO: 139;
(h) an antibody comprising a heavy chain variable region comprising heavy chain CDR1 having the amino acid sequence as set forth in SEQ ID NO: 145, heavy chain CDR2 having the amino acid sequence as set forth in SEQ ID NO: 147, and heavy chain CDR3 having the amino acid sequence as set forth in SEQ ID NO: 149, and a light chain variable region comprising light chain CDR1 having the amino acid sequence as set forth in SEQ ID NO: 155, light chain CDR2 having the amino acid sequence as set forth in SEQ ID NO: 157, and light chain CDR3 having the amino acid sequence as set forth in SEQ ID NO: 159;
(i) an antibody comprising a heavy chain variable region comprising heavy chain CDR1 having the amino acid sequence as set forth in SEQ ID NO: 165, heavy chain CDR2 having the amino acid sequence as set forth in SEQ ID NO: 167, and heavy chain CDR3 having the amino acid sequence as set forth in SEQ ID NO: 169, and a light chain variable region comprising light chain CDR1 having the amino acid sequence as set forth in SEQ ID NO: 175, light chain CDR2 having the amino acid sequence as set forth in SEQ ID NO: 177, and light chain CDR3 having the amino acid sequence as set forth in SEQ ID NO: 179;
(j) an antibody comprising a heavy chain variable region comprising heavy chain CDR1 having the amino acid sequence as set forth in SEQ ID NO: 185, heavy chain CDR2 having the amino acid sequence as set forth in SEQ ID NO: 187, and heavy chain CDR3 having the amino acid sequence as set forth in SEQ ID NO: 189, and a light chain variable region comprising light chain CDR1 having the amino acid sequence as set forth in SEQ ID NO: 195, light chain CDR2 having the amino acid sequence as set forth in SEQ ID NO: 197, and light chain CDR3 having the amino acid sequence as set forth in SEQ ID NO: 199;
(k) an antibody comprising a heavy chain variable region comprising heavy chain CDR1 having the amino acid sequence as set forth in SEQ ID NO: 205, heavy chain CDR2 having the amino acid sequence as set forth in SEQ ID NO: 207, and heavy chain CDR3 having the amino acid sequence as set forth in SEQ ID NO: 209, and a light chain variable region comprising light chain CDR1 having the amino acid sequence as set forth in SEQ ID NO: 215, light chain CDR2 having the amino acid sequence as set forth in SEQ ID NO: 217, and light chain CDR3 having the amino acid sequence as set forth in SEQ ID NO: 219;
(l) an antibody comprising a heavy chain variable region comprising heavy chain CDR1 having the amino acid sequence as set forth in SEQ ID NO: 225, heavy chain CDR2 having the amino acid sequence as set forth in SEQ ID NO: 227, and heavy chain CDR3 having the amino acid sequence as set forth in SEQ ID NO: 229, and a light chain variable region comprising light chain CDR1 having the amino acid sequence as set forth in SEQ ID NO: 235, light chain CDR2 having the amino acid sequence as set forth in SEQ ID NO: 237, and light chain CDR3 having the amino acid sequence as set forth in SEQ ID NO: 239; or
(m) an antibody which binds to the same epitope as that in the human PRDX4 (Peroxiredoxin-4, e.g., UniProtKB—Q13162) protein to which any one of the antibodies (a) to (1) bind.

The present invention further relates to use of the antibody of the present invention in a method for treating diabetes.

### Overview of the Sequence Listing

As described herein and unless otherwise stated references are made to UniProtKB Accession Numbers (https://www.uniprot.org/news/2021/04/07/release, e.g., as available in UniProt release 2021_02, published Apr. 7, 2021).

As described herein unless otherwise stated references are made to NCBI GenBank Accession Numbers (https://www.ncbi.nlm.nih.gov/genbank/release/current/, e.g., as available in Release 243.0, published Apr. 15, 2021).

SEQ ID NO: 1 is the amino acid sequence of the human Endosome/lysosome-associated apoptosis and autophagy regulator 1 (ELAPOR1), which is interchangeably referred herein as “IGFR-L1”, e.g., corresponding to the UniProtKB Accession Number: Q6UXG2-1. An extracellular domain of the IGFR-L1 protein corresponds to amino acids 1-910 of the SEQ ID NO: 1.

SEQ ID NOs: 2-241 are the amino acid sequences of the chains and specific regions of the 12 novel exemplary antibodies of the present invention (Abs, i.e., AB-1, AB-2, AB-3, AB-4, AB-5, AB-6, AB-7, AB-8, AB-9, AB-10, AB-11 and AB-12), wherein Tables 1 showing correspondence of specific SEQ ID NOs to specific heavy chain regions and Table 2 showing correspondence of specific SEQ ID NOs to specific light chain regions.

Table 1 showing correspondence of specific SEQ ID NOs to specific heavy chain regions (“H_{C}”—heavy chain, “L”—leader sequence, Tail region of the heavy chain comprising C_{H}+Fc regions, “HFR”—framework region of the heavy chain, “CDR-H”—complementarity-determining region of the heavy chain):

| | | Leader | | | | | | | | Tail |
|---|---|---|---|---|---|---|---|---|---|---|
| | H_{C} | (L) | HFR1 | CDR-H1 | HFR2 | CDR-H2 | HFR3 | CDR-H3 | HFR4 | region |
| | SEQ | SEQ | SEQ | SEQ | SEQ | SEQ | SEQ | SEQ | SEQ | SEQ |
| | ID | ID | ID | ID | ID | ID | ID | ID | ID | ID |
| | NOs: | NOs: | NOs: | NOs: | NOs: | NOs: | NOs: | NOs: | NOs: | NOs: |
| AB-1 | 2 | 3 | 4 | 5 | 6 | 7 | 8 | 9 | 10 | 11 |
| AB-2 | 22 | 23 | 24 | 25 | 26 | 27 | 28 | 29 | 30 | 31 |
| AB-3 | 42 | 43 | 44 | 45 | 46 | 47 | 48 | 49 | 50 | 51 |
| AB-4 | 62 | 63 | 64 | 65 | 66 | 67 | 68 | 69 | 70 | 71 |
| AB-5 | 82 | 83 | 84 | 85 | 86 | 87 | 88 | 89 | 90 | 91 |
| AB-6 | 102 | 103 | 104 | 105 | 106 | 107 | 108 | 109 | 110 | 111 |
| AB-7 | 122 | 123 | 124 | 125 | 126 | 127 | 128 | 129 | 130 | 131 |
| AB-8 | 142 | 143 | 144 | 145 | 146 | 147 | 148 | 149 | 150 | 151 |
| AB-9 | 162 | 163 | 164 | 165 | 166 | 167 | 168 | 169 | 170 | 171 |
| AB-10 | 182 | 183 | 184 | 185 | 186 | 187 | 188 | 189 | 190 | 191 |
| AB-11 | 202 | 203 | 204 | 205 | 206 | 207 | 208 | 209 | 210 | 211 |
| AB-12 | 222 | 223 | 224 | 225 | 226 | 227 | 228 | 229 | 230 | 231 |

Table 2 showing correspondence of specific SEQ ID NOs to specific light chain regions (“L_{C}”—light chain, “L”—leader sequence or Tail region of the light chain comprising “C_{L}”-constant region, “LFR”—framework region of the light chain, “CDR-L”-complementarity-determining region of the light chain):

| | | | | | | | | | | Tail |
|---|---|---|---|---|---|---|---|---|---|---|
| | | Leader | | | | | | | | Region |
| | Lc | (L) | LFR1 | CDR-L1 | LFR2 | CDR-L2 | LFR3 | CDR-L3 | LFR4 | (C_{L}) |
| | SEQ | SEQ | SEQ | SEQ | SEQ | SEQ | SEQ | SEQ | SEQ | SEQ |
| | ID | ID | ID | ID | ID | ID | ID | ID | ID | ID |
| | NOs: | NOs: | NOs: | NOs: | NOs: | NOs: | NOs: | NOs: | NOs: | NOs: |
| AB-1 | 12 | 13 | 14 | 15 | 16 | 17 | 18 | 19 | 20 | 21 |
| AB-2 | 32 | 33 | 34 | 35 | 36 | 37 | 38 | 39 | 40 | 41 |
| AB-3 | 52 | 53 | 54 | 55 | 56 | 57 | 58 | 59 | 60 | 61 |
| AB-4 | 72 | 73 | 74 | 75 | 76 | 77 | 78 | 79 | 80 | 81 |
| AB-5 | 92 | 93 | 94 | 95 | 96 | 97 | 98 | 99 | 100 | 101 |
| AB-6 | 112 | 113 | 114 | 115 | 116 | 117 | 118 | 119 | 120 | 121 |
| AB-7 | 132 | 133 | 134 | 135 | 136 | 137 | 138 | 139 | 140 | 141 |
| AB-8 | 152 | 153 | 154 | 155 | 156 | 157 | 158 | 159 | 160 | 161 |
| AB-9 | 172 | 173 | 174 | 175 | 176 | 177 | 178 | 179 | 180 | 181 |
| AB-10 | 192 | 193 | 194 | 195 | 196 | 197 | 198 | 199 | 200 | 201 |
| AB-11 | 212 | 213 | 214 | 215 | 216 | 217 | 218 | 219 | 220 | 221 |
| AB-12 | 232 | 233 | 234 | 235 | 236 | 237 | 238 | 239 | 240 | 241 |

SEQ ID NO: 242 is an amino acid sequence depicting an exemplary comparative epitope sequence derived from WO2017042242 (SEQ ID NO: 3 therein) used to raise a comparative domain-specific peptide AB as used herein (e.g., see FIGS. 1-2).

SEQ ID NO: 243 is an amino acid sequence depicting another exemplary comparative epitope sequence derived from WO2017042242 (SEQ ID NO: 4 therein) used to raise a comparative domain-specific peptide AB as used herein (e.g., see FIGS. 1-2).

SEQ ID NO: 244 is an amino acid sequence depicting yet another exemplary comparative epitope sequence of the present invention derived from WO2017042242 (SEQ ID NO: 6 therein) used to raise a comparative domain-specific peptide AB as used herein (e.g., see FIGS. 1-2).

### DESCRIPTION OF THE FIGURES

The invention will be better understood with reference to the detailed description when considered in conjunction with the non-limiting examples and the accompanying drawings. The figures illustrate embodiments of methods of the invention.

FIG. 1: Comparison of the novel structure-specific monoclonal antibody (AB) of the present invention to domain-specific peptide ABs from the prior art under non-denaturing (No DTT and ambient/room temperature) and denaturing conditions (+DTT or +DTT and +95° C.).

The structure specific AB detects the protein under non-denaturing conditions in a SDS gel after Western blotting, whereas the peptide AB only detects the denatured protein. Sequence ID NOs against which the domain-specific peptide ABs were raised from patent application WO2017042242 are given.

FIG. 2: Western blotting showing a comparison of the novel structure-specific monoclonal antibody (AB) of the present invention to domain-specific peptide AB from the prior art on the activation of InsR/IGF1R activation and downstream signaling proteins. Structure-specific AB activate p-InsR/IGF1R, p-IRS1 and p-AKT in a dose-dependent manner, whereas domain-specific peptide antibodies have no or inhibitory effects on InsR/IGF1R pathway activation.

FIG. 3: Western blotting of the AB-1 to AB-12 of the present invention.

FIG. 4: ELISA measurements of the AB-1 to AB-12 of the present invention.

FIG. 5: Kd measurements of antibody affinity of the AB-1 to AB-12 of the present invention.

FIG. 6: Sequence analysis of the light chains of the AB-1 to AB-12 of the present invention. A. mbed-like clustering guide tree. B. Sequence alignment. C. Percent Identity Matrix—created by Clustal2.1.

FIG. 7: Sequence analysis of the heavy chains of the AB-1 to AB-12 of the present invention. A. mbed-like clustering guide tree. B. Sequence alignment. C. Percent Identity Matrix—created by Clustal2.1.

FIG. 8: Min 6 cells preparation and Western blotting of the AB-1 to AB-12 of the present invention showing said ABs are capable of modulating insulin receptor sensitization.

### DETAILED DESCRIPTION

The present inventors produced and characterized novel structure specific IGFR-L1 antibodies for specifically targeting extracellular domain of non-denatured IGFR-L1 protein (SEQ ID NO: 1). This is particularly advantageous as it relates to a new therapeutic method for treating diabetes, preventing or reversing insulin resistance, reversing dysfunction/de-differentiation of pancreatic islet cells and/or increasing the number of pancreatic beta cells. Accordingly, novel structure specific IGFR-L1 antibodies as described herein, are uniquely different from known domain specific peptide AB and are envisaged to define new diabetes patient groups in that they can be used for preventing or reversing insulin resistance, reversing de-differentiation of pancreatic islet cells and/or increasing the number of pancreatic beta cells in diabetes patients. Furthermore, novel structure specific AB of the present invention are capable of detecting their target protein under non-denaturing conditions, e.g., in a SDS gel after Western blotting, whereas the peptide AB known from the prior art only can merely detect the denatured protein; Further, structure-specific AB are capable of activating p-InsR/IGF1R, p-IRS1 and p-AKT in a dose-dependent manner, whereas known domain-specific peptide antibodies have no or inhibitory effects on InsR/IGF1R pathway activation. Therefore, novel structure specific AB of the present invention represent a novel unique class of IGFR-L1 antibodies. This is particularly advantageous the new antibodies of the present invention are suitable for use in immunoprecipitation methods for which domain-specific peptide antibodies from the prior art are not suitable for.

The present inventors pioneered in elucidating the function of the human KIAA1324 gene in encoding an IGFR-like receptor (SEQ ID NO: 1) as described herein. The protein product of KIAA1324 previously referred to as UPF0577 protein KIAA1324 or Estrogen-induced gene 121 (EIG121) protein has commonly been known as a cancer marker, whereas the present inventors for the first time attributed a pivotal role in metabolism to the protein. The terms “IGFR-like receptor”, “IGF-like receptor”; “IGF3 receptor”, “IGF3R”, “ELAPOR1”, “Endosome/lysosome-associated apoptosis and autophagy regulator 1”, “Inceptor”, and “IGFR-L1” are used herein interchangeably herein and refer to Endosome/lysosome-associated apoptosis and autophagy regulator 1, e.g., having UniProtKB Accession Number: Q6UXG2.

In some aspects, the present invention relates to an antibody which binds to the extracellular domain of folded or partially folded Inceptor protein comprising the amino acid sequence of SEQ ID NO: 1, preferably to a native epitope within IGFR-L1 protein, wherein binding to said folded or partially folded IGFR-L1 protein by the antibody is determinable (e.g., determined) follows:
(a) incubating an aliquot of a lysate from Min6 cells (ATCC CRL-11506) separately with each of
   (i) a buffer suitable for SDS-PAGE of proteins for 5 min at ambient temperature,
   (ii) a buffer suitable for SDS-PAGE of proteins and a reducing agent for 5 min at ambient temperature, and
   (iii) a buffer suitable for SDS-PAGE of proteins and a reducing agent for 5 min at 95° C.,
(b) subjecting said aliquots to SDS-PAGE;
(c) western blotting the SDS-PAGE gel;
(d) incubating the western blot membrane with the antibody;
wherein the antibody detects only aliquot (i).

In some further aspects of the present invention, step (a) comprising: incubating an aliquot of a lysate from Min6 cells (ATCC CRL-11506) separately with each of
(i) 50 mM Tris-HCl pH=6.8, 2.5 mM EDTA, 2% (w/v) SDS and 5% (v/v) glycerol, buffer without a thiol agent for 5 min at ambient temperature,
(ii) 50 mM Tris-HCl pH=6.8, 2.5 mM EDTA, 2% (w/v) SDS and 5% (v/v) glycerol, 20 mM DTT for 5 min at ambient temperature, and
(iii) 50 mM Tris-HCl pH=6.8, 2.5 mM EDTA, 2% (w/v) SDS and 5% (v/v) glycerol, 20 mM DTT for 5 min at 95° C.

In some aspects of the present invention, an antibody of the present invention is capable of increasing or decreasing or modulating or inhibiting binding of one or more growth factors (e.g., insulin) or similar ligand/s, morphogen/s, homo-oligomerization partner/s, hetero-oligomerization partner/s and/or di-oligomerization partner/s to Inceptor protein and/or other receptor/s.

In some aspects of the present invention, an antibody of the present invention is capable of increasing binding of insulin to insulin receptor/s (InsR).

In some aspects of the present invention, an antibody of the present invention is capable of modulating binding of insulin to insulin receptor/s (InsR).

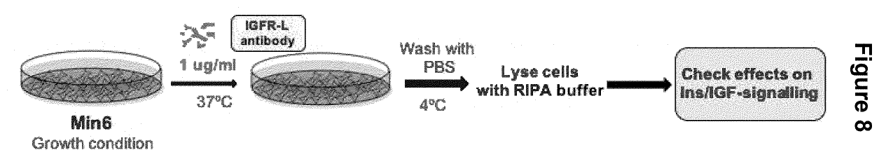

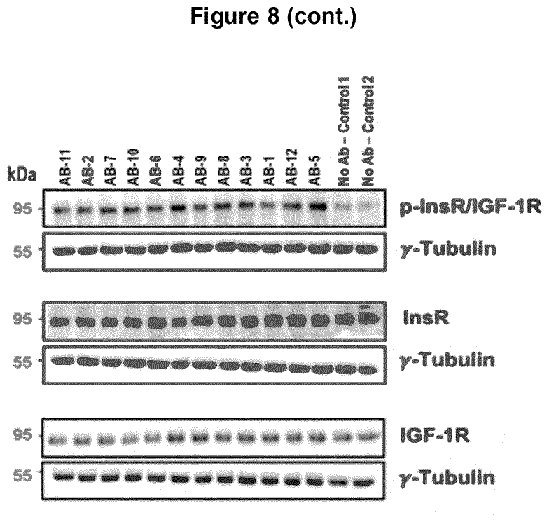

In some aspects of the present invention, all twelve antibodies are generated using a specific method against the native extracellular domain of IGFR-L1/Inceptor (see Examples disclosed herein). Inceptor binds indirectly to the insulin receptor complex at the plasma membrane and mediates internalization and thus desensitization of the activated insulin receptor complex. Therefore, when insulin-producing beta cells are incubated with any one of the 12 antibodies, the antibody binds to the extracellular domain of the inceptor and thus prevents interaction with the activated insulin receptor complex. Thus, the insulin receptor is not desensitized and insulin signal activation is enhanced and/or prolonged (FIG. 8). In other words, the Inceptor Ak of the present invention bind to Inceptor (they all might have a different paratope i.e. epitope) and thereby prevent the Inceptor from efficiently coming together with the activated insulin receptor complex. Thus, the insulin receptor is not efficiently desensitized and remains longer at the plasma membrane and is prolongly activated. This is supported by FIG. 8 for all 12 novel Aks of the present invention (p-IR/IGF1R) and provides a unifying feature for all Aks of the present invention, besides the unique production method.

In some aspects of the present invention, an antibody of the present invention is capable of increasing InsR-mediated signaling.

In some aspects of the present invention, an antibody of the present invention is capable of increasing phosphorylation of InsR, IRS1 and/or AKT and/or MEK- and/or MAP-kinase.

In some aspects of the present invention, an antibody of the present invention is capable of binding to Inceptor protein with a Kd of 10 nM or less, preferably 6 nM or less, more preferably 5 nM or less, even more preferably 3 nM or less.

In some aspects, an antibody of the present invention is obtainable by immunizing a rodent animal with proteoliposomes comprising Inceptor protein.

In some aspects of the present invention, an antibody of the present invention has the following structural characteristics: (a) an antibody comprising a heavy chain having an amino acid sequence with at least 85% identity to the amino acid sequence shown in SEQ ID NO: 2 and a light chain having an amino acid sequence with at least 85% identity to the amino acid sequence shown in SEQ ID NO: 12.

In some aspects of the present invention, an antibody of the present invention has the following structural characteristics: (b) an antibody comprising a heavy chain having an amino acid sequence with at least 85% identity to the amino acid sequence shown in SEQ ID NO: 22 and a light chain having an amino acid sequence with at least 85% identity to the amino acid sequence shown in SEQ ID NO: 32.

In some aspects of the present invention, an antibody of the present invention has the following structural characteristics: (c) an antibody comprising a heavy chain having an amino acid sequence with at least 85% identity to the amino acid sequence shown in SEQ ID NO: 42 and a light chain having an amino acid sequence with at least 85% identity to the amino acid sequence shown in SEQ ID NO: 52.

In some aspects of the present invention, an antibody of the present invention has the following structural characteristics: (d) an antibody comprising a heavy chain having an amino acid sequence with at least 85% identity to the amino acid sequence shown in SEQ ID NO: 62 and a light chain having an amino acid sequence with at least 85% identity to the amino acid sequence shown in SEQ ID NO: 72;

In some aspects of the present invention, an antibody of the present invention has the following structural characteristics: (e) an antibody comprising a heavy chain having an amino acid sequence with at least 85% identity to the amino acid sequence shown in SEQ ID NO: 82 and a light chain having an amino acid sequence with at least 85% identity to the amino acid sequence shown in SEQ ID NO: 92.

In some aspects of the present invention, an antibody of the present invention has the following structural characteristics: (f) an antibody comprising a heavy chain having an amino acid sequence with at least 85% identity to the amino acid sequence shown in SEQ ID NO: 102 and a light chain having an amino acid sequence with at least 85% identity to the amino acid sequence shown in SEQ ID NO: 112.

In some aspects of the present invention, an antibody of the present invention has the following structural characteristics: (g) an antibody comprising a heavy chain having an amino acid sequence with at least 85% identity to the amino acid sequence shown in SEQ ID NO: 122 and a light chain having an amino acid sequence with at least 85% identity to the amino acid sequence shown in SEQ ID NO: 132.

In some aspects of the present invention, an antibody of the present invention has the following structural characteristics: (h) an antibody comprising a heavy chain having an amino acid sequence with at least 85% identity to the amino acid sequence shown in SEQ ID NO: 142 and a light chain having an amino acid sequence with at least 85% identity to the amino acid sequence shown in SEQ ID NO: 152.

In some aspects of the present invention, an antibody of the present invention has the following structural characteristics: (i) an antibody comprising a heavy chain having an amino acid sequence with at least 85% identity to the amino acid sequence shown in SEQ ID NO: 162 and a light chain having an amino acid sequence with at least 85% identity to the amino acid sequence shown in SEQ ID NO: 172.

In some aspects of the present invention, an antibody of the present invention has the following structural characteristics: (j) an antibody comprising a heavy chain having an amino acid sequence with at least 85% identity to the amino acid sequence shown in SEQ ID NO: 182 and a light chain having an amino acid sequence with at least 85% identity to the amino acid sequence shown in SEQ ID NO: 192.

In some aspects of the present invention, an antibody of the present invention has the following structural characteristics: (k) an antibody comprising a heavy chain having an amino acid sequence with at least 85% identity to the amino acid sequence shown in SEQ ID NO: 202 and a light chain having an amino acid sequence with at least 85% identity to the amino acid sequence shown in SEQ ID NO: 212.

In some aspects of the present invention, an antibody of the present invention has the following structural characteristics: (l) an antibody comprising a heavy chain having an amino acid sequence with at least 85% identity to the amino acid sequence shown in SEQ ID NO: 222 and a light chain having an amino acid sequence with at least 85% identity to the amino acid sequence shown in SEQ ID NO: 232.

In some aspects of the present invention, an antibody of the present invention has the following structural characteristics: (m) an antibody which binds to the same epitope as that in Inceptor protein to which any one of the antibodies (a) to (l) as defined herein bind.

In some aspects of the present invention, an antibody of the present invention has the following structural characteristics: (a) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 19 to 140 of SEQ ID NO: 2 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 28 to 136 of SEQ ID NO: 12.

In some aspects of the present invention, an antibody of the present invention has the following structural characteristics: (b) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 62 to 177 of SEQ ID NO: 22 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 48 to 162 of SEQ ID NO: 32.

In some aspects of the present invention, an antibody of the present invention has the following structural characteristics: (c) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 20 to 136 of SEQ ID NO: 42 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 39 to 153 of SEQ ID NO: 52.

In some aspects of the present invention, an antibody of the present invention has the following structural characteristics: (d) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 20 to 141 of SEQ ID NO: 62 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 40 to 148 of SEQ ID NO: 72.

In some aspects of the present invention, an antibody of the present invention has the following structural characteristics: (e) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 58 to 175 of SEQ ID NO: 82 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 21 to 129 of SEQ ID NO: 92.

In some aspects of the present invention, an antibody of the present invention has the following structural characteristics: (f) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 54 to 171 of SEQ ID NO: 102 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 55 to 163 of SEQ ID NO: 112.

In some aspects of the present invention, an antibody of the present invention has the following structural characteristics: (g) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 19 to 139 of SEQ ID NO: 122 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 45 to 156 of SEQ ID NO: 132.

In some aspects of the present invention, an antibody of the present invention has the following structural characteristics: (h) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 20 to 139 of SEQ ID NO: 142 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 40 to 148 of SEQ ID NO: 152.

In some aspects of the present invention, an antibody of the present invention has the following structural characteristics: (i) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 20 to 133 of SEQ ID NO: 162 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 40 to 148 of SEQ ID NO: 172.

In some aspects of the present invention, an antibody of the present invention has the following structural characteristics: (j) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 54 to 175 of SEQ ID NO: 182 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 38 to 149 of SEQ ID NO: 192.

In some aspects of the present invention, an antibody of the present invention has the following structural characteristics: (k) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 55 to 170 of SEQ ID NO: 202 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 21 to 135 of SEQ ID NO: 212.

In some aspects of the present invention, an antibody of the present invention has the following structural characteristics: (l) an antibody comprising a heavy chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 20 to 144 of SEQ ID NO: 222 and a light chain variable region having an amino acid sequence with at least 80% (e.g., at least, 85%, 86%, 87%, 88%, 89%, 90%, 91%, 92%, 93%, 94%, 95%, 96%, 97%, 98%, 99% or 100%) identity to the amino acids 52 to 165 of SEQ ID NO: 232.

In some aspects of the present invention, an antibody of the present invention has the following structural characteristics: (m) an antibody which binds to the same epitope as that in Inceptor protein to which any one of the antibodies (a) to (l) bind.

In some aspects of the present invention, an antibody of the present invention has one or more of the following structural characteristics: (a) an antibody comprising a heavy chain variable region comprising heavy chain CDR1 having the amino acid sequence as set forth in SEQ ID NO: 5, heavy chain CDR2 having the amino acid sequence as set forth in SEQ ID NO: 7, and heavy chain CDR3 having the amino acid sequence as set forth in SEQ ID NO: 9 and a light chain variable region comprising light chain CDR1 having the amino acid sequence as set forth in SEQ ID NO: 15, light chain CDR2 having the amino acid sequence as set forth in SEQ ID NO: 17, and light chain CDR3 having the amino acid sequence as set forth in SEQ ID NO: 19.

In some aspects of the present invention, an antibody of the present invention has one or more of the following structural characteristics: (b) an antibody comprising a heavy chain variable region comprising heavy chain CDR1 having the amino acid sequence as set forth in SEQ ID NO: 25, heavy chain CDR2 having the amino acid sequence as set forth in SEQ ID NO: 27, and heavy chain CDR3 having the amino acid sequence as set forth in SEQ ID NO: 29, and a light chain variable region comprising light chain CDR1 having the amino acid sequence as set forth in SEQ ID NO: 35, light chain CDR2 having the amino acid sequence as set forth in SEQ ID NO: 37, and light chain CDR3 having the amino acid sequence as set forth in SEQ ID NO: 39.

In some aspects of the present invention, an antibody of the present invention has one or more of the following structural characteristics: (c) an antibody comprising a heavy chain variable region comprising heavy chain CDR1 having the amino acid sequence as set forth in SEQ ID NO: 45, heavy chain CDR2 having the amino acid sequence as set forth in SEQ ID NO: 47, and heavy chain CDR3 having the amino acid sequence as set forth in SEQ ID NO: 49, and a light chain variable region comprising light chain CDR1 having the amino acid sequence as set forth in SEQ ID NO: 55, light chain CDR2 having the amino acid sequence as set forth in SEQ ID NO: 57, and light chain CDR3 having the amino acid sequence as set forth in SEQ ID NO: 59.

In some aspects of the present invention, an antibody of the present invention has one or more of the following structural characteristics: (d) an antibody comprising a heavy chain variable region comprising heavy chain CDR1 having the amino acid sequence as set forth in SEQ ID NO: 65, heavy chain CDR2 having the amino acid sequence as set forth in SEQ ID NO: 67, and heavy chain CDR3 having the amino acid sequence as set forth in SEQ ID NO: 69, and a light chain variable region comprising light chain CDR1 having the amino acid sequence as set forth in SEQ ID NO: 75, light chain CDR2 having the amino acid sequence as set forth in SEQ ID NO: 77, and light chain CDR3 having the amino acid sequence as set forth in SEQ ID NO: 79.

In some aspects of the present invention, an antibody of the present invention has one or more of the following structural characteristics: (e) an antibody comprising a heavy chain variable region comprising heavy chain CDR1 having the amino acid sequence as set forth in SEQ ID NO: 85, heavy chain CDR2 having the amino acid sequence as set forth in SEQ ID NO: 87, and heavy chain CDR3 having the amino acid sequence as set forth in SEQ ID NO: 89, and a light chain variable region comprising light chain CDR1 having the amino acid sequence as set forth in SEQ ID NO: 95, light chain CDR2 having the amino acid sequence as set forth in SEQ ID NO: 97, and light chain CDR3 having the amino acid sequence as set forth in SEQ ID NO: 99.

In some aspects of the present invention, an antibody of the present invention has one or more of the following structural characteristics: (f) an antibody comprising a heavy chain variable region comprising heavy chain CDR1 having the amino acid sequence as set forth in SEQ ID NO: 105, heavy chain CDR2 having the amino acid sequence as set forth in SEQ ID NO: 107, and heavy chain CDR3 having the amino acid sequence as set forth in SEQ ID NO: 109, and a light chain variable region comprising light chain CDR1 having the amino acid sequence as set forth in SEQ ID NO: 115, light chain CDR2 having the amino acid sequence as set forth in SEQ ID NO: 117, and light chain CDR3 having the amino acid sequence as set forth in SEQ ID NO: 119.

In some aspects of the present invention, an antibody of the present invention has one or more of the following structural characteristics: (g) an antibody comprising a heavy chain variable region comprising heavy chain CDR1 having the amino acid sequence as set forth in SEQ ID NO: 125, heavy chain CDR2 having the amino acid sequence as set forth in SEQ ID NO: 127, and heavy chain CDR3 having the amino acid sequence as set forth in SEQ ID NO: 129, and a light chain variable region comprising light chain CDR1 having the amino acid sequence as set forth in SEQ ID NO: 135, light chain CDR2 having the amino acid sequence as set forth in SEQ ID NO: 137, and light chain CDR3 having the amino acid sequence as set forth in SEQ ID NO: 139;

In some aspects of the present invention, an antibody of the present invention has one or more of the following structural characteristics: (h) an antibody comprising a heavy chain variable region comprising heavy chain CDR1 having the amino acid sequence as set forth in SEQ ID NO: 145, heavy chain CDR2 having the amino acid sequence as set forth in SEQ ID NO: 147, and heavy chain CDR3 having the amino acid sequence as set forth in SEQ ID NO: 149, and a light chain variable region comprising light chain CDR1 having the amino acid sequence as set forth in SEQ ID NO: 155, light chain CDR2 having the amino acid sequence as set forth in SEQ ID NO: 157, and light chain CDR3 having the amino acid sequence as set forth in SEQ ID NO: 159.

In some aspects of the present invention, an antibody of the present invention has one or more of the following structural characteristics: (i) an antibody comprising a heavy chain variable region comprising heavy chain CDR1 having the amino acid sequence as set forth in SEQ ID NO: 165, heavy chain CDR2 having the amino acid sequence as set forth in SEQ ID NO: 167, and heavy chain CDR3 having the amino acid sequence as set forth in SEQ ID NO: 169, and a light chain variable region comprising light chain CDR1 having the amino acid sequence as set forth in SEQ ID NO: 175, light chain CDR2 having the amino acid sequence as set forth in SEQ ID NO: 177, and light chain CDR3 having the amino acid sequence as set forth in SEQ ID NO: 179.

In some aspects of the present invention, an antibody of the present invention has one or more of the following structural characteristics: (j) an antibody comprising a heavy chain variable region comprising heavy chain CDR1 having the amino acid sequence as set forth in SEQ ID NO: 185, heavy chain CDR2 having the amino acid sequence as set forth in SEQ ID NO: 187, and heavy chain CDR3 having the amino acid sequence as set forth in SEQ ID NO: 189, and a light chain variable region comprising light chain CDR1 having the amino acid sequence as set forth in SEQ ID NO: 195, light chain CDR2 having the amino acid sequence as set forth in SEQ ID NO: 197, and light chain CDR3 having the amino acid sequence as set forth in SEQ ID NO: 199.

In some aspects of the present invention, an antibody of the present invention has one or more of the following structural characteristics: (k) an antibody comprising a heavy chain variable region comprising heavy chain CDR1 having the amino acid sequence as set forth in SEQ ID NO: 205, heavy chain CDR2 having the amino acid sequence as set forth in SEQ ID NO: 207, and heavy chain CDR3 having the amino acid sequence as set forth in SEQ ID NO: 209, and a light chain variable region comprising light chain CDR1 having the amino acid sequence as set forth in SEQ ID NO: 215, light chain CDR2 having the amino acid sequence as set forth in SEQ ID NO: 217, and light chain CDR3 having the amino acid sequence as set forth in SEQ ID NO: 219.

In some aspects of the present invention, an antibody of the present invention has one or more of the following structural characteristics: (l) an antibody comprising a heavy chain variable region comprising heavy chain CDR1 having the amino acid sequence as set forth in SEQ ID NO: 225, heavy chain CDR2 having the amino acid sequence as set forth in SEQ ID NO: 227, and heavy chain CDR3 having the amino acid sequence as set forth in SEQ ID NO: 229, and a light chain variable region comprising light chain CDR1 having the amino acid sequence as set forth in SEQ ID NO: 235, light chain CDR2 having the amino acid sequence as set forth in SEQ ID NO: 237, and light chain CDR3 having the amino acid sequence as set forth in SEQ ID NO: 239.

In some aspects of the present invention, an antibody of the present invention has one or more of the following structural characteristics: (m) an antibody which binds to the same epitope as that in the human PRDX4 protein to which any one of the antibodies (a) to (l) bind.

In some aspects of the present invention, an antibody of the present invention is for use as a medicament and/or in therapy.

In some aspects of the present invention, an antibody of the present invention is for use in a method for treating diabetes and/or hyperinsulinism.

In some aspects of the present invention, diabetes comprises type 1 diabetes, type 2 diabetes, gestational diabetes, prediabetes, insulin resistance, metabolic syndrome or glucose tolerance or hyperinsulinism.

In some aspects of the present invention, an antibody of the present invention is for use in a method of preventing or reversing insulin resistance.

In some aspects of the present invention, an antibody of the present invention is for use in a method of reversing dysfunction/de-differentiation of pancreatic islet cells.

In some aspects of the present invention, an antibody of the present invention is for use in a method of increasing the number of pancreatic beta cells.

In some aspects of the present invention, the natural/independent selection/generation of two individual clones with conserved variable chain sequences (e.g., clones corresponding to AB-2 and AB-11), but differences in the Fc chain highlight that highly conserved/available epitope/co-epitope/antigen and its importance in receptor structure/function regulation; importance of Fc domain sequence and modification thereof, mutation and/or chemical modification such as PEGylation, Glycosylation etc., for optimizing antibody stability and bio-availability.

Functional variants of the IGFR-like receptor disclosed herein, which have a threshold sequence identity or sequence homology to the IGFR-like receptor described herein, are also encompassed by the term “IGFR-like receptor”. Said functional variants are envisaged to have at least 50%, 55%, 60%, 65%, 70%, 75%, 80%, 85%, 90%, 95%, 97%, 98%, 99%, 99.5% or 100% sequence identity with SEQ ID NO: 1.

It is further envisioned that said functional variants preferably exhibit the same properties as UPF0577 protein KIAA1324, i.e. inhibit or reduce InsR and/or IGF1R-mediated signaling, in particular (i) Akt phosphorylation and/or (ii) AMPK phosphorylation and/or (iii) mTOR phosphorylation as assessable by routine methods set out in the appended examples.

The term “% identity” or “% sequence identity” as used herein refers to the percentage of pair-wise identical residues—following (homologous) alignment of a sequence of a polypeptide of the invention with a sequence in question—with respect to the number of residues in the longer of these two sequences. Percent identity is determined by dividing the number of identical residues by the total number of residues and multiplying the product by 100.

The term “homology” is used herein in its usual meaning and includes identical amino acids as well as amino acids which are regarded to be conservative substitutions (for example, exchange of a glutamate residue by an aspartate residue) at equivalent positions in the linear amino acid sequence of two proteins. Preferably, the amino acid sequence shown in SEQ ID NO: 182 or 192 are preferred as a “reference sequence”.

SEQ ID NO: 1 shows the human UPF0577 protein KIAA1324, also referred to as IGFR-like receptor herein.

The term “reference sequence” and “wild type sequence” (of the IGFR-like receptor) is used interchangeably herein. Alternatively, the amino acid sequence with the SWISS-PROT/UniProt Data Bank Accession Number Q6UXG2 (entry version 95 of 22 Jul. 2015) can be used as reference sequence.

The percentage of sequence homology or sequence identity can, for example, be determined herein using the BLASTP, version blastp 2.2.5 (Nov. 16, 2002; cf. Altschul, S. F. et al. (1997) Nucl. Acids Res. 25, 3389-3402). In this embodiment the percentage of homology is based on the alignment of the entire polypeptide sequences (matrix: BLOSUM 62; gap costs: 11.1) including the propeptide sequences, preferably using the wild type protein scaffold as reference in a pairwise comparison. It is calculated as the percentage of numbers of “positives” (homologous amino acids) indicated as result in the BLASTP program output divided by the total number of amino acids selected by the program for the alignment.

In the context of the invention, the expression “position corresponding to another position” (e.g., regions, fragments, nucleotide or amino acid positions, or the like) is based on the convention of numbering according to nucleotide or amino acid position number and then aligning the sequences in a manner that maximizes the percentage of sequence identity. Because not all positions within a given “corresponding region” need be identical, non-matching positions within a corresponding region may be regarded as “corresponding positions.” Accordingly, as used herein, referral to an “amino acid position corresponding to amino acid position [X]” of a specified protein sequence represents, in addition to referral to amino acid positions of the specified protein sequence, referral to a collection of equivalent positions in other recognized protein and structural homologues and families. The same may be applied to the expression “sequence corresponding to sequence”, mutatis mutandis. I.e., the referral to a sequence “corresponding to” a specified protein sequence [X], in addition to referral to sequence the specified protein sequence, referral to a collection of equivalent sequences in other recognized protein and structural homologues and families.

The term “InsR” or “IR” refers to the insulin receptor and generally comprises both the IR-A (also known as “isoform short”) and IR-B (also known as “isoform long”) isoforms. The InsR occurs as a tetramer of 2 α chains carrying the insulin-binding regions and 2 β chains carrying the kinase domain, linked by disulfide bonds. The InsR is a receptor tyrosine kinase that is activated by binding of insulin, IGF-1 and IGF-2, ultimately leading to signaling through the MAPK/Ras-Raf-Erk pathway, the phosphatidylinositol-3-kinase/AKT/mTOR (PI3K/AKT) pathway and/or the Janus kinase/signal transducer and activator of transcription (JAK/STAT) pathway. More precisely, ligand binding to the α-chains of the InsR ectodomain induces structural changes within the receptor leading to autophosphorylation of various tyrosine residues within the intracellular tyrosine kinase domain of the β-chain, leading to recruitment and phosphorylation of several intracellular substrates, including, insulin receptor substrates (IRS1, 2, 3, 4), SHC, GAB1, CBL and other signaling intermediates. Each of these phosphorylated proteins serve as docking proteins for other signaling proteins that contain Src-homology-2 domains (SH2 domain), including the p85 regulatory subunit of PI3K and SHP2. Phosphorylation of IRSs proteins leads to the activation of two main signaling pathways: the PI3K-AKT/PKB pathway, which is responsible for most of the metabolic actions of insulin, and the Ras-MAPK pathway, which regulates expression of some genes and cooperates with the PI3K pathway to control cell growth and differentiation. Binding of PI3K to phosphotyrosines on IRS1 leads and subsequent PI3K activation leads to the phosphorylation and activation of AKT, AMPK and mTOR, a signaling pathway which regulates metabolism and integrates signals from insulin. InsR activation upon ligand binding also triggers the Ras/RAF/MAP2K/MAPK pathway via phosphorylation of IRS1 and recruitment of GRB2/SOS, which is mainly involved in mediating cell growth, survival and cellular differentiation of insulin.

An illustrative example of the InsR is the human InsR with Uniprot Acc. No. P06213 (entry version 216 of Jul. 22, 2015) and variants thereof. Insulin receptors in the context of the present invention are preferably capable of inducing (i) AKT phosphorylation, and/or (ii) AMPK phosphorylation and/or (iii) mTOR phosphorylation upon binding of their ligand, in particular insulin.

The term “IGF-receptor 1” or “IGF1R” or “IGFRI” is used herein to refer to the Insulin-like growth factor 1 receptor tyrosine kinase. IGF1R binds to IGF1 with high affinity and IGF2 and insulin (INS) with a lower affinity. Ligand binding activates the receptor kinase, leading to receptor autophosphorylation, and phosphorylation of multiple substrates, including, the insulin-receptor substrates (IRS1/2), Shc and 14-3-3 proteins, which ultimately leads to the activation of three main signaling pathways: the PI3K-AKT/PKB pathway, the Ras-MAPK pathway, and the JAK/STAT pathway. The activated IGF1R is involved in cell growth and survival control. Thus, although InsR and IGF1R feed into similar signaling pathways, InsR-mediated signaling predominantly regulates metabolism, whereas IGF1R signaling is involved in cell growth and survival.

An illustrative example of the IGF1R is the human IGF1R with Uniprot Acc. No. P08069 (entry version 185 of Jul. 22, 2015) and variants thereof. IGF1R in the context of the present invention is preferably capable of inducing (i) AKT phosphorylation, and/or (ii) AMPK phosphorylation and/or (iii) mTOR phosphorylation upon binding of their ligand, in particular IGF1.

The term “IGF1” or “IGFI” refers to the Insulin-like growth factor I, a protein structurally and functionally related to insulin, but having a higher growth-promoting activity. An illustrative example is the human IGF1 with Uniprot Acc. No. P05019 (entry version 186 of Jul. 22, 2015). “IGF1” in the context of the present invention is preferably capable of binding to the IGF1 receptor and eliciting IGF1R signaling as described elsewhere herein.

The term “IGF2R” or “IGF2R” or “IGFRII” is used herein to refer to the Insulin-like growth factor 2/mannose-6-phosphate (IGF-2/M6P) receptor. The IGF2R is a single transmembrane protein composed of a large extracytoplasmic (i.e. extracellular) domain, a single transmembrane region and a short cytoplasmic tail that lacks intrinsic catalytic activity. The receptor binds IGF-2 with higher affinity than IGF-1 and does not bind insulin. The IGF2R has been reported to interact, via distinct sites, with lysosomal enzymes and a variety of other M6P-containing ligands, and regulate extracellular IGF-2 concentrations, thereby modulating signaling through the growth-stimulatory IGF-1 receptor pathway.

An illustrative example of the IGF2R is the human IGF2R with Uniprot Acc. No. P11717 (entry version 174 of Jul. 22, 2015) and variants thereof.

The term “IGF2” or “IGFII” refers to the Insulin-like growth factor II. An illustrative example is the human IGF2 with Uniprot Acc. No. P01344 (entry version 199 of Jul. 22, 2015) and variants thereof. “IGF2” in the context of the present invention is preferably capable of binding to the IGF2 receptor.

As used herein a variant or ortholog of the human KIAA1324 gene is envisaged to encode an IGFR-like receptor or a functional variant thereof, i.e. preferably being capable of inhibiting or reducing InsR and/or IGF1R-mediated signaling, in particular (i) AKT phosphorylation and/or (ii) AMPK phosphorylation and/or (iii) mTOR phosphorylation and having at least about 60%, 65%, 70%, 75%, 80%, 90%, 95%, 97%, 98%, 99%, 99.5% or 100% sequence identity with the human KIAA1324 gene (NCBI Gene ID 57535, updated on 15 Jul. 2015), i.e. sequence identity with the human KIAA1324 gene's coding sequence shown in SEQ ID NO: 7. The coding sequence is obtained by joining nucleotides 222 . . . 374, 47932 . . . 48052, 50537 . . . 50729, 57904 . . . 58051, 58526 . . . 58606, 59512 . . . 59617, 59726 . . . 59875, 71083.71171, 74215 . . . 74392, 75104.75232, 75630.75720, 77404 . . . 77509, 77764 . . . 77901, 78649 . . . 78912, 80459 . . . 80632, 83512 . . . 83692, 84017 . . . 84113, 84611 . . . 84712, 85892 . . . 86018, 86097 . . . 86275, 86773 . . . 86938, 88982 . . . 89050 of said human KIAA1324 gene (NG_032763.1).

As used herein the term “isolated DNA sequence” refers to a DNA molecule purified, or substantially purified, from endogenous material, including other nucleic acid sequences, proteins, peptides, lipids and so on naturally occurring in the cell and/or organism from which the DNA sequence is derived and includes DNA purified by standard purification techniques as well as DNA prepared by recombinant technology and those chemically synthesized.

As used herein the term “folded or partially folded” may mean having 3D or tertiary structure with or without post-translational modification, e.g., one or more alpha helixes, turns, unfolded elements, intrinsically disordered elements, coil-coil elements, beta-sheets and/or individual structural elements thereof.

As used herein the term “tertiary structure” may mean having three dimensional shape of a polypeptide in space comprising one or more protein secondary structures and/or the protein domains with or without post-translational modification, e.g., one or more alpha helixes, turns, unfolded elements, intrinsically disordered elements, coil-coil elements, beta-sheets and/or individual structural elements thereof.

As used herein the term “morphogen” may mean a substance whose non-uniform distribution governs the pattern of tissue development in the process of morphogenesis or pattern formation.

As used herein the term “homo-oligomerization partner/s” may mean identical monomers comprised within a polymer or protein complex (e.g., protein complex comprising Inceptor protein).

As used herein the term “hetero-oligomerization partner/s” may mean non-identical monomers comprised within a polymer or protein complex (e.g., protein complex comprising Inceptor protein).

As used herein the term “di- or oligomerization partner/s” may mean identical or non-identical monomers comprised within a dimer or oligomer (e.g., protein complex comprising Inceptor protein).

### Vector

The nucleic acid of the invention may also be in the form of, may be present in and/or may be part of a vector.

The term “vector” refers a nucleic acid molecule used as a vehicle to transfer (foreign) genetic material into a host cell and encompasses—without limitation—plasmids, viruses, cosmids and artificial chromosomes such as bacterial artificial chromosomes (BACs) and yeast artificial chromosomes (YACs). In general, engineered vectors comprise an origin of replication, a multicloning site and a selectable marker. The vector itself is generally a nucleotide sequence, commonly a DNA sequence that comprises an insert (transgene) and a larger sequence that serves as the “backbone” of the vector. Vectors may encompass additional elements besides the transgene insert and a backbone including gene regulation elements, genetic markers, antibiotic resistances, reporter genes, targeting sequences, or protein purification tags. Particularly envisaged within the context of the invention are expression vectors (expression constructs) for expression of the transgene in the host cell, which generally comprise—in addition to the transgene—gene regulation sequences.

An expression vector is, in general, a vector that can provide for expression of the antibodies of the present invention in vitro and/or in vivo (i.e. in a suitable host cell, host organism and/or expression system). The person skilled in the art will readily understand that choice of a particular vector include depends, e.g., on the host cell, the intended number of copies of the vector, whether transient or stable expression of the antibody of the present invention is envisaged, and so on.

“Transient expression” results from the introduction of a nucleic acid (e.g. a linear or non-linear DNA or RNA molecule) or vector that is incapable of autonomous replication into a recipient host cell. Expression of the transgene occurs through the transient expression of the introduced sequence.

However, “stable expression” of the nucleic acid sequence as described herein will often be preferred and may be accomplished by either stably integrating the nucleic acid sequence into the host cell's genome or by introducing a vector comprising the nucleic acid sequence of the invention and being capable of autonomously replicating into the host cell.

The vector provided herein is in particular envisaged to comprise a gene regulation element operably linked to the DNA sequence encoding antibody of the present invention.

The term “gene regulation element” refers to DNA sequences necessary for the expression of an operably linked coding sequence in a particular host organism. The term “gene regulation element” includes controllable transcriptional promoters, operators, enhancers, silencers, transcriptional terminators, 5′ and 3′ untranslated regions which interact with host cellular proteins to carry out transcription and translation and other elements that may control gene expression including initiation and termination codons. The precise nature of the regulatory regions needed for gene expression may vary from organism to organism. Prokaryotic gene regulation elements, for example, include a promoter, optionally an operator sequence, and a ribosome binding site (RBS), whereas gene regulation elements for eukaryotic cells comprise promoters, polyadenylation (poly-A) signals, and enhancers.

The gene regulation element is envisaged to be “operably linked” to the gene to be expressed, i.e. placed in functional relationship with the same. For instance, a promoter or enhancer is “operably linked” to a coding nucleic acid sequence if it affects the transcription of the sequence. The DNA sequences being “operably linked” may or may not be contiguous. Linking is typically accomplished by ligation at convenient restriction sites or synthetic oligonucleotide adaptors or linkers.

### Host Cell

Further provided herein is a host cell (e.g., recombinant and/or isolated host cell) comprising the vector as described herein.

A variety of host cells can be employed for expressing the nucleic acid sequence encoding antibodies as described herein. Host cells can be prepared using genetic engineering methods known in the art. The process of introducing the vector into a recipient host cell is also termed “transformation” or “transfection” hereinafter. The terms are used interchangeably herein.

Host cell transformation typically involves opening transient pores or “holes” in the cell wall and/or cell membrane to allow the uptake of material. Illustrative examples of transformation protocols involve the use of calcium phosphate, electroporation, cell squeezing, dendrimers, liposomes, cationic polymers such as DEAE-dextran or polyethylenimine, sonoporation, optical transfection, impalefection, nanoparticles (gene gun), magnetofection, particle bombardement, alkali cations (cesium, lithium), enzymatic digestion, agitation with glass beads, viral vectors, or others. The choice of method is generally dependent on the type of cell being transformed, the vector to be introduced into the cell and the conditions under which the transformation is taking place.

As used herein, the term “host cell” refers to any cell or cell culture acting as recipients for the vector or isolated nucleic acid sequence encoding the Abs as described herein. Suitable host cells include prokaryotic or eukaryotic cells, and also include but are not limited to bacteria, yeast cells, fungi cells, plant cells, and animal cells such as insect cells and mammalian cells, e.g., murine, rat, macaque or human.

E.g., the Abs can be produced in bacteria. In addition to prokaryotes, eukaryotic microbes such as filamentous fungi or yeast are suitable cloning or expression hosts for the IGFR-like receptor of the invention. Illustrative examples include Saccharomyces cerevisiae, Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of glycosylated antibody construct of the invention may also be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa californica NPV and the Bm-5 strain of Bombyx mori NPV.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art.

Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/-DHFR (CHO), mouse sertoli cells (TM4); monkey kidney cells (CVI ATCC CCL 70); African green monkey kidney cells (VERO-76, ATCC CRL1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2,1413 8065); mouse mammary tumor (MMT 060562, ATCC CCL5 1); TRI cells; MRC 5 cells; FS4 cells; and human hepatoma cells (Hep G2).

### Antibody

The antibodies provided herein preferably exhibit the desired biological activity, i.e. specifically bind to the extracellular domain of non-denatured Inceptor protein described herein. “Increase” thereby denotes an increase in the respective signal in the presence of the antibody when compared to the absence of the antibody in the respective detection method which is used for the detection and/or quantification of said increase.

As is well known in the art, an antibody is an immunoglobulin molecule capable of specific binding to a target (epitope) through at least one epitope recognition site, located in the variable region of the immunoglobulin molecule. The term “antibody” as used herein comprises monoclonal and polyclonal antibodies, as well as (naturally occurring or synthetic) fragments or variants thereof, including fusion proteins, multimeric assemblies (e.g., IgG-dimer etc.) comprising an antibody portion with an antigen-binding fragment of the required specificity and any other modified configuration of the antibody that comprises an antigen-binding site or fragment (epitope recognition site) of the required specificity. Illustrative examples include dAb, nanobody, affibody, Fab, Fab′, F(ab′)₂, Fv, single chain Fvs (scFv), diabodies, and minibodies comprising a scFv joined to a CH3 domain. It will be understood that other antibody frameworks or scaffolds comprising “antigen-binding sites” can be employed in line with the present invention. The term “antibody” thus also comprises these scaffolds. The mentioned scaffolds include e.g. non-immunoglobulin based antibodies and scaffolds onto which CDRs of the antibodies can be grafted. Such scaffolds include for example anticalins, avimers, affilins etc.

The antibody may be a chimeric antibody (or antigen-binding variant or fragment thereof). The term “chimeric antibody” refers to an antibody in which a portion of the heavy and/or light chain is identical with or homologous to corresponding sequences in antibodies derived from a particular species or belonging to a particular antibody class or subclass, while the remainder of the chain (s) is identical with or homologous to corresponding sequences in antibodies derived from another species or belonging to another antibody class or subclass, as well as fragments of such antibodies.

The antibody may be a humanized antibody (or antigen-binding variant or fragment thereof). The term “humanized antibody” refers to an antibody containing a minimal sequence derived from a non-human antibody. In general, humanized antibodies are human immunoglobulins comprising residues from a hypervariable region of an immunoglobulin derived from non-human species such as mouse, rat, rabbit or non-human primate (“donor antibody”) grafted onto the human immunoglobulin (“recipient antibody”). In some instances, framework region (FR) residues of the human immunoglobulin are replaced by corresponding non-human residues. Furthermore, humanized antibodies may comprise residues that are neither found in the recipient antibody nor in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

The antibody may be a human antibody. A “human antibody” is one that possesses an amino-acid sequence corresponding to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol Biol, 227:381 (1991); Marks et al, J. Mol Biol, 222:581 (1991)). Also available for the preparation of human monoclonal antibodies are methods described in Cole et al, Monoclonal Antibodies and Cancer Therapy, Alan R. Liss, p. 77 (1985) and Boerner et al., J. Immunol, 147(1):86-95 (1991). See also van Dijk and van de Winkel, Curr. Opin. Pharmacol, 5: 368-374 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al, Proc. Natl. Acad. Sci. USA, 103:3557-3562 (2006) regarding human antibodies generated via a human B-cell hybridoma technology.

### Chemical Modifications

The antibodies or antigen-binding variants or fragments thereof used in accordance with of the invention may be modified. Typical modifications conceivable in the context of the invention include, e.g., chemical modifications as described in the following.

Generally, all kind of modifications are conceivable as long as they do not abolish the capability of the antibodies or antigen-binding variants or fragments thereof to specifically bind to the extracellular domain of non-denatured Inceptor protein as described elsewhere herein.

Possible chemical modifications of the antibody or antigen-binding variants or fragments thereof include acylation or acetylation of the amino-terminal end or amidation or esterification of the carboxy-terminal end or, alternatively, on both. The modifications may also affect the amino group in the side chain of lysine or the hydroxyl group of threonine. Other suitable modifications include, e.g., extension of an amino group with polypeptide chains of varying length (e.g., XTEN technology or PASylation®), N-glycosylation, O-glycosylation, and chemical conjugation of carbohydrates, such as hydroxyethyl starch (e.g., HESylation®) or polysialic acid (e.g., PolyXen® technology). Chemical modifications such as alkylation (e. g., methylation, propylation, butylation), arylation, and etherification may be possible and are also envisaged.

The antibodies of the present invention specifically bind to the extracellular domain of non-denatured Inceptor protein comprising the amino acid sequence of SEQ ID NO: 1, preferably to a native epitope within IGFR-L1 protein, i.e. do not to exhibit cross-reactivity towards non-target molecules such as InsR, the IGF1R and IGF2R.

The term “epitope” in general refers to a site on an antigen to which a binding domain, such as an antibody or immunoglobulin or derivative or fragment of an antibody or of an immunoglobulin, specifically binds. An “epitope” is antigenic and thus the term epitope is sometimes also referred to herein as “antigenic structure” or “antigenic determinant”. Thus, the binding domain is an “antigen interaction site”. Said binding/interaction is also understood to define a “specific recognition”. The term “epitope” encompasses linear epitopes and conformational epitopes. Linear epitopes are contiguous epitopes comprised in the amino acid primary sequence and typically include at least 3 or at least 4, and more usually, at least 5 or at least 6 or at least 7, for example, about 8 to about 10 amino acids in a unique sequence. Conformational epitopes are formed by non-contiguous amino acids juxtaposed by folding of the protein. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

The term “polypeptide” and “protein” may be used interchangeably herein.

### Specific Binding

The terms “binding to” and “recognizing” in all grammatical forms are used interchangeably herein.

The term “specifically binds” generally indicates that a binding agent, in particular an antibody, binds with higher affinity to its intended target (i.e. to the extracellular domain of non-denatured Inceptor protein described herein) than to its non-target molecule. Non-target molecules include the IGF receptors, in particular the human IGF1R with Uniprot Acc. No. P08069 (entry version 185 of 22 Jul. 2015), the human IGF2R with Uniprot Acc. No. P11717 (entry version 174 of 22 Jul. 2015), and the human InsR with Uniprot Acc. No. P06213 (entry version 216 of 22 Jul. 2015); and functional variants thereof. Preferably the affinity of the agonist or antagonist will be at least about 5 fold, preferably 10 fold, more preferably 25-fold, even more preferably 50-fold, and most preferably 100-fold or more, greater for a target molecule than its affinity for a non-target molecule. Preferred antibodies bind with affinities of at least about 10⁷ M⁻¹, and preferably between about 10⁸ M⁻¹ to about 10⁹ M⁻¹, about 10⁹ M⁻¹ to about 1010 M⁻¹, or about 1010 M⁻¹ to about 1012 M⁻¹.

Preferably, the term “specifically binds” thus indicates that an antagonist or agonist, such as an antibody, exclusively binds to its intended target (i.e., the IGFR-like receptor).

### Treatment

It is envisaged that the IGFR-like receptor antibodies of the present invention are particularly useful in treatment and diagnostic of diabetes. As used herein, “diabetes” refers to the broad class of disorders characterized by impaired insulin production and glucose tolerance and in general includes type 1 and type 2 diabetes (also called juvenile and adult-onset, respectively), gestational diabetes, prediabetes, insulin resistance, metabolic syndrome, and impaired glucose tolerance. Diabetes results from a deficiency or functional impairment of insulin-producing β cells, alone or in combination with insulin resistance.

The term “metabolic syndrome” comprises abdominal (central) obesity, elevated blood pressure, elevated fasting plasma glucose, high serum triglycerides, low high-density lipoprotein (HDL) and/or high low-density lipoprotein (LDL) levels. Further it is associated with the risk of developing type 2 diabetes and/or cardiovascular disease including coronary heart diseases.

The term “β cell(s)”, “beta cell(s)” and “islet cell(s)” are used interchangeably herein to refer to the pancreatic β cells located in the islet of Langerhans. Their primary function is to store and release insulin.

Defective insulin secretion underlies all forms of diabetes mellitus. Whereas the destruction of β-cells is responsible for type 1 diabetes (T1 D), both lowered β-cell mass and loss of secretory function are implicated in type 2 diabetes (T2D). Emerging results suggest that a functional deficiency, involving de-differentiation of the mature β-cell towards a more progenitor-like state, may be an important driver for impaired secretion in T2D.

It is contemplated that the antagonists and agonists of the present invention can advantageously be employed in preventing β cell de-differentiation and/or reverting β cell loss-of-function. The antagonist and agonists described herein are therefore envisaged for use as a medicament. Particularly, said antagonists and agonists are intended for use in a method of prophylactic and/or therapeutic treatment of diabetes.

Type 1 diabetes is also known as Insulin Dependent Diabetes Mellitus (IDDM), and juvenile diabetes. The terms are used interchangeably herein. This form accounts for 5-10% of diabetes and is thought to be due to cellular-mediated autoimmune destruction of the pancreatic β-cells, resulting in little or no insulin secretion. Antagonists and agonists of the IGFR-like receptor provided herein may be capable of preventing or even reverting β cell de-differentiation and/or loss-of-function. Thus, the present invention is envisaged to open up new possibilities for a preventive or regenerative therapy of T1D.

Type 2 diabetes is also referred to as adult-onset diabetes and accounts for ˜90-95% of all diabetes. Insulin resistance in target tissues and a relative deficiency of insulin secretion from pancreatic β-cells are the major features of type 2 diabetes (T2D). Insulin resistance is used herein to denote a condition characterized by the failure of target cells to respond to insulin, leading to hyperglycemia. Pancreatic β cells in the pancreas subsequently increase their production of insulin, leading to hyperinsulinemia. Without wishing to be bound by specific theory, it is contemplated the IGFR-like receptor described herein acts as a scavenger for either insulin or the insulin receptor. E.g., the IGFR-like receptor may bind to the insulin receptor, leading to internalization of the same (insulin receptor scavenger). The IGFR-like receptor may also bind to insulin, leading to its internalization and potentially lysosomal degradation (insulin scavenger). By inhibiting these IGFR-like receptor functions with the help of antagonists as described herein, InsR- and/or IGF1R-mediated signaling may increase, thereby restoring insulin sensitivity.

### Patients

The term “patient” or “subject” as used herein refers to a human or non-human animal, generally a mammal. Particularly envisaged is a mammal, such as a rabbit, a mouse, a rat, a Guinea pig, a hamster, a dog, a cat, a pig, a cow, a goat, a sheep, a horse, a monkey, an ape or preferably a human. Thus, the methods, uses and compounds described in this document are in general applicable to both human and veterinary disease.

### Treatment

The term “treatment” in all its grammatical forms includes therapeutic or prophylactic treatment. A “therapeutic or prophylactic treatment” comprises prophylactic treatments aimed at the complete prevention of clinical and/or pathological manifestations or therapeutic treatment aimed at amelioration or remission of clinical and/or pathological manifestations of the diseases. The term “treatment” thus also includes the amelioration or prevention of diabetes.

In the context with the present invention the term “therapeutic effect” in general refers to the desirable or beneficial impact of a treatment, e.g. amelioration or remission of the disease manifestations. The term “manifestation” of a disease is used herein to describe its perceptible expression, and includes both clinical manifestations, hereinafter defined as indications of the disease that may be detected during a physical examination and/or that are perceptible by the patient (i.e., symptoms), and pathological manifestations, meaning expressions of the disease on the cellular and molecular level. The therapeutic effect of treatment with the IGFR-like antibodies of the present invention can be assessed using routine methods in the art, e.g. measuring insulin levels and/or glucose levels in blood samples of the patient. Additionally or alternatively, it is also possible to evaluate the general appearance of the respective patient (e.g., fitness, well-being) which will also aid the skilled practitioner to evaluate whether a therapeutic effect has been elicited. The skilled person is aware of numerous other ways which are suitable to observe a therapeutic effect of the compounds of the present invention.

### Dose

Preferably, a therapeutically effective amount of the compound as described herein is administered. By “therapeutically effective amount” is meant an amount of the compound as described herein that elicits a therapeutic effect. The exact dose of IGFR-like receptor antibodies of the present invention will depend on the purpose of the treatment (e.g. remission maintenance vs. treatment of acute flare of the disease), and will be ascertainable by one skilled in the art using known techniques. Adjustments for route of administration, age, body weight, general health, sex, diet, time of administration, drug interaction and the severity of the condition may be necessary, and will be ascertainable with routine experimentation by those skilled in the art.

### Administration

A variety of routes are applicable for administration of the compound according to the present invention, including, but not limited to, orally, topically, transdermally, subcutaneously, intravenously, intraperitoneally, intramuscularly or intraocularly. However, any other route may readily be chosen by the person skilled in the art if desired.

### Composition

It is envisaged to administer the IGFR-like antibodies of the present invention in the form of a pharmaceutical composition.

The term “pharmaceutical composition” particularly refers to a composition suitable for administering to a human, i.e., a composition that is preferably sterile and/or contains components which are pharmaceutically acceptable. However, compositions suitable for administration to non-human animals are also envisaged herein. Preferably, a pharmaceutical composition comprises an IGFR-like antibodies of the present invention together with one or more pharmaceutical excipients. The term “excipient” includes fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers or tonicity modifiers. Pharmaceutical compositions of the invention can be formulated in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration.

The pharmaceutical composition of the present invention may further comprise one or more additional agents. Preferably, said agents are therapeutically effective for treatment the diseases described herein and present in the composition in a therapeutically effective amount. Examples include, without limitation, metformin, sulfonylureas, meglitinides, thiazolidinediones, DPP-4 inhibitors, GLP-1 receptor agonists, SGLT2 inhibitors, insulin and insulin derivatives (insulin glulisine, insulin lispro, insulin aspart, insulin glargine, insulin detemir, insulin isophane), and combinations thereof.

In view of the above, the present invention hence also provides a pharmaceutical composition comprising one or more IGFR-L1 antibodies of the present invention. Said pharmaceutical composition is particularly intended for use in a method of therapeutic and/or prophylactic treatment of diabetes.

### Kit

A kit is also provided herein. The kit may be a kit of two or more parts, and comprises the IGFR-like receptor antibodies of the present invention, preferably in a therapeutically effective amount and in a pharmaceutically acceptable form. The components of the kit may be contained in a container or vials. The kit is envisaged to comprise additional agents useful in treating diabetes, as described elsewhere herein. Exemplary additional agents include, without limitation, metformin, sulfonylureas, meglitinides, thiazolidinediones, DPP-4 inhibitors, GLP-1 receptor agonists, SGLT2 inhibitors, insulin and insulin derivatives (insulin glulisine, insulin lispro, insulin aspart, insulin glargine, insulin detemir, insulin isophane), and combinations thereof.

The IGFR-like antibodies of the present invention and the additional agents can be administered simultaneously or sequentially to the patient.

The present invention is also characterized by the following items:
1. An antibody (e.g., an antigen binding portion thereof) which binds to the extracellular domain of non-denatured (e.g. native) Inceptor protein comprising the amino acid sequence of SEQ ID NO: 1, preferably to a native epitope within IGFR-L1 protein, further preferably wherein binding to non-denatured IGFR-L1 protein by the antibody is determinable (e.g., determined) as follows:
   (a) incubating an aliquot of a lysate from Min6 cells (ATCC CRL-11506) separately with each of
      (i) a buffer suitable for SDS-PAGE of proteins for 5 min at ambient temperature,
      (ii) a buffer suitable for SDS-PAGE of proteins and a reducing agent for 5 min at ambient temperature, and
      (iii) a buffer suitable for SDS-PAGE of proteins and a reducing agent for 5 min at 95° C.,
   (b) subjecting said aliquots to SDS-PAGE;
   (c) western blotting the SDS-PAGE gel;
   (d) incubating the western blot membrane with the antibody;
   wherein the antibody detects only aliquot (i).
2. An antibody which binds to the extracellular domain of a folded (e.g., native) or partially folded (e.g., having 3D and/or tertiary structure with or without post-translational modification, e.g., one or more alpha helixes, turns, unfolded elements, intrinsically disordered elements, coil-coil elements, beta-sheets and/or individual structural elements thereof) Inceptor protein comprising the amino acid sequence of SEQ ID NO: 1, preferably to a native epitope within IGFR-L1 protein, wherein binding to said folded or partially folded IGFR-L1 protein by the antibody is determinable (e.g., determined) as follows:
   (a) incubating an aliquot of a lysate from Min6 cells (ATCC CRL-11506) separately with each of
      (i) a buffer suitable for SDS-PAGE of proteins for 5 min at ambient temperature,
      (ii) a buffer suitable for SDS-PAGE of proteins and a reducing agent for 5 min at ambient temperature, and
      (iii) a buffer suitable for SDS-PAGE of proteins and a reducing agent for 5 min at 95° C.,
   (b) subjecting said aliquots to SDS-PAGE;
   (c) western blotting the SDS-PAGE gel;
   (d) incubating the western blot membrane with the antibody;
   wherein the antibody detects only aliquot (i).
3. The antibody of any one of the preceding items, wherein said partially unfolded is carried out by the means of SDS-treatment according to any one of the preceding items.
4. The antibody of any one of the preceding items, wherein step (a) comprises
   (a) incubating an aliquot of a lysate from Min6 cells (ATCC CRL-11506) separately with each of
      (i) 50 mM Tris-HCl pH=6.8, 2.5 mM EDTA, 2% (w/v) SDS and 5% (v/v) glycerol, buffer without a thiol agent for 5 min at ambient temperature,
      (ii) 50 mM Tris-HCl pH=6.8, 2.5 mM EDTA, 2% (w/v) SDS and 5% (v/v) glycerol, 20 mM DTT for 5 min at ambient temperature, and
      (iii) 50 mM Tris-HCl pH=6.8, 2.5 mM EDTA, 2% (w/v) SDS and 5% (v/v) glycerol, 20 mM DTT for 5 min at 95° C.
5. The antibody of any one of the preceding items, wherein the antibody increases or decreases or modulates or inhibits binding of one or more growth factors (e.g., insulin) or similar ligand/s, morphogen/s, homo-oligomerization partner/s, hetero-oligomerization partner/s and/or di-oligomerization partner/s to Inceptor protein and/or other receptor/s (e.g., InsR).
6. The antibody of any one of the preceding items, wherein the antibody increases or decreases or modulates or inhibits binding of insulin to insulin receptor/s (e.g., InsR).
7. The antibody of any one of the preceding items, wherein the antibody increases binding of insulin to insulin receptor/s (e.g., InsR).
8. The antibody of any one of the preceding items, wherein the antibody increases insulin receptor sensitivity.
9. The antibody of any one of the preceding items, wherein the antibody modulates insulin receptor sensitivity.
10. The antibody of any one of the preceding items, wherein the antibody modulates insulin on- and off-rate binding kinetics and/or de- and hypersensitation of InsR kinase domain.
11. The antibody of any one of the preceding items, wherein the antibody increases InsR-mediated signaling, preferably said increase is in comparison to a control antibody of the same class and/or subtype.
12. The antibody of any one of the preceding items, wherein the antibody increases phosphorylation of InsR, IRS1 and/or AKT and/or MEK- and/or MAP-kinase, preferably said increase is in comparison to a control antibody of the same class and/or subtype.
13. The antibody of any one of the preceding items, wherein said extracellular domain ranges from amino acids 1 to 910 of SEQ ID NO: 1, preferably said extracellular domain ranges from amino acids 42 to 910 of SEQ ID NO: 1 (e.g., is devoid of the signaling peptide having amino acids 1 to 41 of SEQ ID NO: 1).
14. The antibody of any one of the preceding items, wherein the antibody binds to Inceptor protein with a Kd of 10 nM or less, preferably 6 nM or less, more preferably 5 nM or less, even more preferably 3 nM or less.
15. The antibody of any one of the preceding items, wherein the antibody is producible by immunizing a rodent animal with proteoliposomes comprising Inceptor protein.
16. The antibody of any one of the preceding items, wherein the antibody is
   (a) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 2 and a light chain having an amino acid sequence with at least 25% (e.g., at least, 80% 85%, 86%, 87%, 88%, 89%, 90%, 91%, 92%, 93%, 94%, 95%, 96%, 97%, 98%, 99% or 100%) identity to the amino acid sequence shown in SEQ ID NO: 12;
   (b) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 22 and a light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 32;
   (c) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 42 and a light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 52;
   (d) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 62 and a light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 72;
   (e) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 82 and a light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 92;
   (f) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 102 and a light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 112;
   (g) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 122 and a light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 132;
   (h) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 142 and a light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 152;
   (i) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 162 and a light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 172;
   (j) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 182 and a light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 192;
   (k) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 202 and a light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 212;
   (l) an antibody comprising a heavy chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 222 and a light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence shown in SEQ ID NO: 232; or
   (m) an antibody which binds to the same epitope as that in Inceptor protein to which any one of the antibodies (a) to (l) bind.
17. The antibody of any one of the preceding items, wherein the antibody is
   (a) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 19 to 140 of SEQ ID NO: 2 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 28 to 136 of SEQ ID NO: 12;
   (b) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 62 to 177 of SEQ ID NO: 22 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 48 to 162 of SEQ ID NO: 32;
   (c) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 20 to 136 of SEQ ID NO: 42 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 39 to 153 of SEQ ID NO: 52;
   (d) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 20 to 141 of SEQ ID NO: 62 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 40 to 148 of SEQ ID NO: 72;
   (e) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 58 to 175 of SEQ ID NO: 82 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 21 to 129 of SEQ ID NO: 92;
   (f) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 54 to 171 of SEQ ID NO: 102 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 55 to 163 of SEQ ID NO: 112;
   (g) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 19 to 139 of SEQ ID NO: 122 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 45 to 156 of SEQ ID NO: 132;
   (h) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 20 to 139 of SEQ ID NO: 142 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 40 to 148 of SEQ ID NO: 152;
   (i) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 20 to 133 of SEQ ID NO: 162 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 40 to 148 of SEQ ID NO: 172;
   (j) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 54 to 175 of SEQ ID NO: 182 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 38 to 149 of SEQ ID NO: 192;
   (k) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 55 to 170 of SEQ ID NO: 202 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 21 to 135 of SEQ ID NO: 212;
   (l) an antibody comprising a heavy chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 20 to 144 of SEQ ID NO: 222 and a light chain variable region having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acids 52 to 165 of SEQ ID NO: 232; or
   (m) an antibody which binds to the same epitope as that in Inceptor protein to which any one of the antibodies (a) to (l) bind.
18. The antibody of any one of the preceding items, wherein said variable region/s comprising or consisting of: seven amino acid regions, four of which are corresponding framework regions (e.g., HFRs and/or LFRs, e.g., as depicted in Table 1 and/or Table 2 herein) and three of which are corresponding CDRs (also may be referred to as “hypervariable regions” herein), e.g., as depicted in Table 1 and/or Table 2 herein.
19. The antibody of any one of the preceding items, wherein the antibody is
   (a) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 5, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 7, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 9 and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 15, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 17, and light chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 19;
   (b) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 25, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 27, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 29, and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 35, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 37, and light chain having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 39;
   (c) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 45, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 47, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 49, and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 55, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 57, and light chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 59;
   (d) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 65, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 67, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 69, and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 75, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 77, and light chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 79;
   (e) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 85, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 87, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 89, and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 95, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 97, and light chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 99;
   (f) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 105, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 107, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 109, and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 115, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 117, and light chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 119;
   (g) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 125, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 127, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 129, and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 135, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 137, and light chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 139;
   (h) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 145, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 147, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 149, and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 155, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 157, and light chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 159;
   (i) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 165, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 167, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 169, and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 175, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 177, and light chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 179;
   (j) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 185, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 187, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 189, and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 195, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 197, and light chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 199;
   (k) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 205, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 207, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 209, and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 215, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 217, and light chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 219;
   (l) an antibody comprising a heavy chain variable region comprising heavy chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 225, heavy chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 227, and heavy chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 229, and
      a light chain variable region comprising light chain CDR1 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 235, light chain CDR2 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 237, and light chain CDR3 having an amino acid sequence with at least 25% (e.g., at least, 80%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 239; or
   (m) an antibody which binds to the same epitope as that in the human Peroxiredoxin-4 protein (PRDX4, e.g., having UniProtKB Accession Number: Q13162) to which any one of the antibodies (a) to (l) bind.
20. The antibody of any one of the preceding items, wherein said antibody comprising one or more amino acid sequences with at least 25% (e.g., at least, 80%, 85%, 86%, 87%, 88%, 89%, 90%, 91%, 92%, 93%, 94%, 95%, 96%, 97%, 98%, 99% or 100%) identity to any amino acid sequence shown in SEQ ID NO: 2-241.
21. The antibody of any one of the preceding items, wherein said antibody comprising one or more:
   i) one or more H_{C} (heavy chain) and/or L_{C} (light chain) leader sequences (L), e.g., as depicted in Table 1 and/or Table 2 herein;
   ii) one or more Framework regions HFR1 and/or LFR1, e.g., as depicted in Table 1 and/or Table 2 herein;
   iii) one or more Framework regions HFR2 and/or LFR2, e.g., as depicted in Table 1 and/or Table 2 herein;
   iv) one or more Framework regions HFR3 and/or LFR3, e.g., as depicted in Table 1 and/or Table 2 herein;
   v) one or more Framework regions HFR4 and/or LFR4, e.g., as depicted in Table 1 and/or Table 2 herein; and/or
   vi) one or more tail regions, e.g., H_{L} (heavy chain constant region) and/or C_{L} (light chain constant), e.g., as depicted in Table 1 and/or Table 2 herein.
22. The antibody of any one of the preceding items, wherein said antibody comprising one or more of the following characteristics:
   i) an amino acid I at a position corresponding to the position 104 of SEQ ID NO: 182 (e.g., amino acid sequence of the H_{C} of the AB-10), preferably said amino acid is part of a heavy chain CDR2 region;
   ii) an amino acid T at a position corresponding to the position 111 of SEQ ID NO: 182 (e.g., amino acid sequence of the H_{C} of the AB-10), preferably said amino acid is part of a heavy chain CDR2 region;
   iii) an amino acid T at a position corresponding to the position 137 of SEQ ID NO: 192 (e.g., amino acid sequence of the L_{C} of the AB-10), preferably said amino acid is part of a light chain CDR3 region;
   iv) a heavy chain having an amino acid sequence with at least 72% (e.g., at least, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 1;
   v) a light chain having an amino acid sequence with at least 59% (e.g., at least, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 12;
   vi) a light chain variable region comprising light chain CDR1 (e.g., CDR-11) having an amino acid sequence with at least 50% (e.g., at least, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 15;
   vii) a light chain variable region comprising light chain CDR2 (e.g., CDR-L2) having an amino acid sequence with at least 50% (e.g., at least, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 17;
   viii) a light chain variable region comprising light chain CDR3 (e.g., CDR-L3) having an amino acid sequence with at least 50% (e.g., at least, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 19;
   ix) a heavy chain variable region comprising heavy chain CDR1 (e.g., CDR-H1) having an amino acid sequence with at least 50% (e.g., at least, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 5;
   x) a heavy chain variable region comprising heavy chain CDR2 (e.g., CDR-H2) having an amino acid sequence with at least 50% (e.g., at least, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 7;
   xi) a heavy chain variable region comprising heavy chain CDR3 (e.g., CDR-H3) having an amino acid sequence with at least 50% (e.g., at least, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) identity to the amino acid sequence set forth in SEQ ID NO: 9.
23. The antibody of any one of the preceding items, wherein said antibody comprises one or more (e.g., 2 to 469) conservative amino acid substitution/s (e.g., exemplary or preferred) of the original amino acid/s according to Table 3 below:

| Original | Exemplary substitution(s) - | Preferred substitution(s) - |
|---|---|---|
| amino acid | conservative | conservative |
| Ala (A) | Val (V) | Val (V) |
| (neutral | (non-polar and small) | (nonpolar relatively small) |
| and small) | Leu (L) | Gly (G) |
| | (nonpolar and relatively small) | (neutral and small) |
| | Ile (I) | |
| | (nonpolar and relatively small) | |
| | Gly (G) | |
| | (neutral and small) | |
| Arg (R) | Lys (K) | Lys (K) |
| (polar and | (polar and relatively large) | (polar and relatively large) |
| relatively | Gln (Q) | |
| large) | (polar and relatively small) | |
| | Asn (N) | |
| | (polar and relatively small) | |
| Asn (N) | Gln (Q) | Gln (Q) |
| (polar and | (polar and relatively small) | (polar and relatively small) |
| relatively | His (H) | |
| small) | (polar and relatively large) | |
| | Asp (D) | |
| | (polar and relatively small) | |
| | Lys (K) | |
| | (polar and relatively large) | |
| | Arg (R) | |
| | (polar and relatively large) | |
| Asp (D) | Glu (E) | Glu (E) |
| (negative | (polar and relatively small) | (polar and relatively small) |
| charged | Asn (N) | |
| side chain) | (polar and relatively small) | |
| Cys (C) | Ser (S) | Ser (S) |
| (special) | (neutral and small) | (neutral and small) |
| | Ala (A) | |
| | (neutral and small) | |
| Gln (Q) | Asn (N) | Asn (N) |
| (polar and | (polar and relatively small) | (polar and relatively small) |
| relatively | Glu (E) | |
| small) | (polar and relatively small) | |
| Glu (E) | Asp (D) | Asp (D) |
| (polar and | (polar and relatively small) | (polar and relatively small) |
| relatively | Gln (Q) | |
| small) | (polar and relatively small) | |
| Gly (G) | Ala (A) | Ala (A) |
| (neutral and small) | (neutral and small) | (neutral and small) |
| His (H) | Asn (N) | Arg (R) |
| (polar and | (polar and relatively small) | (polar and relatively large) |
| relatively | Gln (Q) | |
| large) | (polar and relatively small) | |
| | Lys (K) | |
| | (polar and relatively large) | |
| | Arg (R) | |
| | (polar and relatively large) | |
| Ile (I) | Leu (L) | Leu (L) |
| (nonpolar | (nonpolar relatively small) | (nonpolar relatively small) |
| relatively | Val (V) | |
| small) | (nonpolar relatively small) | |
| | Met (M) | |
| | (nonpolar relatively small) | |
| | Ala (A) | |
| | (neutral and small) | |
| | Phe (F) | |
| | (nonpolar and relatively large) | |
| Leu (L) | Norl | Ile (I) |
| (nonpolar | Ile (I) | (nonpolar relatively small) |
| and | (nonpolar relatively small) | |
| relatively | Val (V) | |
| small) | (nonpolar relatively small) | |
| | Met (M) | |
| | (nonpolar relatively small) | |
| | Ala (A) | |
| | (neutral and small) | |
| Lys (K) | Arg (R) | Arg (R) |
| (polar and | (polar and relatively large) | (polar and relatively large) |
| relatively | Gln (Q) | |
| large) | (polar and relatively small) | |
| | Asn (N) | |
| | (polar and relatively small) | |
| Met (M) | Leu (L) | Leu (L) |
| (nonpolar | (nonpolar and relatively small) | (nonpolar and relatively |
| and | Phe (F) | small) |
| relatively | (nonpolar and relatively large) | |
| small) | Ile (I) | |
| | (nonpolar relatively small) | |
| Phe (F) | Leu (L) | Tyr (Y) |
| (nonpolar | (nonpolar and relatively small) | (nonpolar and relatively large) |
| and | Val (V) | |
| relatively | (nonpolar relatively small) | |
| large) | Ile (I) | |
| | (nonpolar relatively small) | |
| | Ala (A) | |
| | (neutral and small) | |
| | Tyr (Y) | |
| | (nonpolar and relatively large) | |
| Pro (P) | Ala (A) | Ala (A) |
| (neutral and small) | (neutral and small) | (neutral and small) |
| Ser (S) | Thr (T) | Thr (T) |
| (neutral and small) | (neutral and small) | (neutral and small) |
| Thr (T) | Ser (S) | Ser (S) |
| (neutral and small) | (neutral and small) | (neutral and small) |
| Trp (W) | Tyr (Y) | Tyr (Y) |
| (nonpolar and | (nonpolar and relatively large) | (nonpolar and relatively large) |
| relatively | Phe (F) | |
| large) | (nonpolar and relatively large) | |
| Tyr (Y) | Trp (W) | Phe (F) |
| (nonpolar | (nonpolar and relatively large) | (nonpolar and relatively large) |
| and | Phe (F) | |
| relatively | (nonpolar and relatively large) | |
| large) | Thr (T) | |
| | (neutral and small) | |
| | Ser (S) | |
| | (neutral and small) | |
| Val (V) | Ile (I) | Leu (L) |
| (non-polar | (nonpolar relatively small) | (nonpolar and relatively |
| and | Leu (L) | small) |
| small) | (nonpolar and relatively small) | |
| | Met (M) | |
| | (nonpolar relatively small) | |
| | Phe (F) | |
| | (nonpolar and relatively large) | |
| | Ala (A) | |
| | (neutral and small) | |

24. The antibody of any one of the preceding items, wherein said antibody is a monoclonal antibody.
25. The antibody of any one of the preceding items, wherein said antibody is chimeric, humanized or human.
26. The antibody of any one of the preceding items, wherein said antibody is coupled to a labelling group and/or protein-Tag and/or comprises one or more substitutions with non-naturally occurring amino acid/s.
27. The antibody of any one of the preceding items, wherein said antibody said antibody is obtainable by a hybridoma (e.g., said antibody is a recombinant antibody).
28. The antibody of any one of the preceding items, wherein said antibody is for use as a medicament and/or in therapy.
29. The antibody of any one of the preceding items, wherein said antibody is for use in a method for treating diabetes and/or cancer.
30. The antibody for the use of any one of the preceding items, wherein diabetes comprises type 1 diabetes, type 2 diabetes, gestational diabetes, prediabetes, insulin resistance, metabolic syndrome or glucose tolerance.
31. The antibody of any one of the preceding items, wherein said antibody is for use in a method of preventing or reversing insulin resistance.
32. The antibody of any one of the preceding items, wherein said antibody is for use in a method of reversing de-differentiation and/or dysfunction of pancreatic islet cells.
33. The antibody of any one of the preceding items, wherein said antibody is for use in a method of increasing the number of pancreatic beta cells.
34. The antibody according to any one of preceding items, wherein said antibody is obtainable by a hybridoma and/or by any other suitable mammalian cell expression system/s.
35. The antibody according to any one of preceding items, wherein said antibody is capable of specifically binding to an ectodomain within SEQ ID NO: 1.
36. The antibody according to any one of preceding items, wherein said antibody is selected from the group consisting of or corresponding to: AB-1, AB-2, AB-3, AB-4, AB-5, AB-5, AB-6, AB-7, AB-8, AB-9, AB-10, AB-11 and AB-12, preferably AB-2 and AB-11.
37. The antibody according to any one of preceding items, wherein said antibody is selected from the group consisting of or corresponding to: AB-2 and AB-11, wherein said AB-2 and AB-11 are obtainable from two individual clones with conserved (e.g., identical) variable chain sequences according to according to any one of preceding items but having differences (e.g., non-identical sequences) in the fragment crystallizable region (i.e., Fc region or Tail-region).
38. The antibody according to any one of preceding items, wherein said antibody is AB-10.
39. The antibody according to any one of preceding items, wherein said antibody is AB-11.
40. The antibody according to any one of preceding items, wherein said antibody is AB-2.
41. The antibody according to any one of preceding items, wherein the Tail region of said antibody is post-translationally modified (e.g., glycosylated, e.g., N- and O-linked glycosylated, glycated, cysteinylated and sulfated; chain trimmed, e.g., C-terminal lysine clipping; amino acid modified, e.g., cyclization (e.g., into a N-terminal pyroglutamic acid), deamidated, oxidated, isomerized and carbamylated; to disulfide scrambling of hinge region interchain disulfide bond, PEGylated, methylated, acetylated and/or phosphorylated etc.), preferably wherein said Tail region comprising: (i) Fc and C_{H} according to any one of preceding items; and/or C_{L} according to any one of preceding items.
42. A multimeric assembly of antibodies (e.g., an IgG-dimer) comprising one or more antibodies according to any one of preceding items.
43. An epitope, epitope, co-epitope or antigen of the antibody according to any one of preceding items, which obtained by the means of one or more of the following: epitope mapping, cryo-electron microscopy, protein crystallization, chemical crosslinking, mass spectrometry, HDX-exchange mass spectrometry, NMR and other suitable structural methods, phage display and/or similar methods, preferably fragments and/or isolated receptor ectodomains as well as complete receptor in cell membranes and/or receptor in detergent micelles or mixed micelles are screened.
44. A hybridoma, wherein said hybridoma produces the monoclonal antibody according to any one of preceding items.
45. A nucleic acid encoding the antibody according to any one of preceding items.
46. An expression vector comprising at least one of the nucleic acid molecules according to any one of preceding items.
47. A bispecific molecule comprising the antibody of any one of the preceding items linked to a molecule having a second binding specificity, wherein said second binding specificity is different from binding specificity of said antibody.
48. An immune-conjugate comprising the antibody of any one of the preceding items linked to a second agent, wherein said second agent is different from said antibody.
49. A host cell (e.g., a recombinant host cell, e.g., an isolated recombinant host cell) comprising a vector and/or nucleic acid according to any one of the preceding items, preferably said host cell is transformed with said vector and/or nucleic acid, further preferably said host cell is a heterologous host cell, further most preferably said host cell is a non-human host cell.
50. A composition comprising the antibody, hybridoma, nucleic acid, expression vector, bispecific molecule, immune-conjugate, host cell, multimeric assembly according to any one of preceding items.
51. The composition according to any one of preceding items, wherein said composition is a pharmaceutical or diagnostic composition.
52. A method for production of the antibody according to any one of preceding items, comprising culturing the host cell according to any one of preceding items under conditions allowing synthesis of said antibody and recovering said antibody or antigen binding portion thereof from said culture.
53. The method according to any one of preceding items, wherein said antibody is a monoclonal or polyclonal antibody.
54. A method of treatment, amelioration, prophylaxis or diagnostics of diabetes comprising administering to a subject in need thereof (e.g., human) a therapeutically effective amount of the antibody, bispecific molecule, multimeric assembly of antibodies or immune-conjugate according to any one of preceding items to treat the diabetes.
55. The method according to any one of preceding items, wherein said diabetes comprises type 1 diabetes, type 2 diabetes, gestational diabetes, prediabetes, insulin resistance, metabolic syndrome or glucose tolerance.
56. A method of preventing or reversing insulin resistance comprising administering to a subject in need thereof (e.g., human) a therapeutically effective amount of the antibody, bispecific molecule, multimeric assembly of antibodies or immune-conjugate according to any one of preceding items to prevent or reverse insulin resistance.
57. A method of reversing dysfunction and/or de-differentiation of pancreatic islet cells comprising administering to a subject in need thereof (e.g., human) a therapeutically effective amount of the antibody, bispecific molecule, multimeric assembly of antibodies or immune-conjugate according to any one of preceding items to reverse said dysfunction and/or de-differentiation of pancreatic islet cells.
58. A method of increasing the number of pancreatic beta cells comprising administering to a subject in need thereof (e.g., human) a therapeutically effective amount of the antibody, bispecific molecule, multimeric assembly of antibodies or immune-conjugate according to any one of preceding items to increase the number of pancreatic beta cells.
59. The method according to any one of preceding items, wherein said method is an in vitro, ex vivo or in vivo method or combinations thereof.
60. A kit comprising the antibody, hybridoma, nucleic acid, expression vector, bispecific molecule, immune-conjugate, host cell, multimeric assembly of antibodies or composition according to any one of preceding items; and optionally, instructions for use of said kit.
61. The antibody, hybridoma, nucleic acid, expression vector, bispecific molecule, immune-conjugate, host cell, multimeric assembly of antibodies or composition according to any one of preceding items, for use as a medicament and/or in therapy.
62. The antibody, hybridoma, nucleic acid, expression vector, bispecific molecule, immune-conjugate, host cell, multimeric assembly of antibodies or composition according to any one of preceding items, for use in one or more of the following methods:
   i) in a method of treatment, amelioration, prophylaxis or diagnostics of diabetes or related disorders (e.g., Hyperinsulinism, insulin resistance and related disorders);
   ii) in a method of preventing or reversing insulin resistance;
   iii) in a method of reversing dysfunction/de-differentiation of pancreatic islet cells;
   iv) in a method of increasing the number of pancreatic beta cells;
   v) in a method for production or preparation of an antibody;
   vi) in a method for immunizing a non-human animal;
   vii) in a method for preparation of a hybridoma;
   viii) in a method for immunoprecipitation (e.g., “pull-down” assay) and/or in a method for identification of epitope or co-epitope or antigen by the means of one or more of the following: epitope mapping, cryo-electron microscopy, protein crystallization, chemical crosslinking, mass spectrometry, HDX—exchange mass spectrometry, NMR and other suitable structural methods, phage display and/or similar methods;
   ix) in a method according to any one of preceding items;
   x) in any one of (i)-(viii), wherein said method is in vitro, ex vivo or in vivo.
63. Use of the antibody, hybridoma, nucleic acid, expression vector, bispecific molecule (e.g., recombinant protein), immune-conjugate, host cell, multimeric assembly of antibodies or fragments thereof or composition according to any one of preceding items for one or more of the following:
   i) for treating diabetes and/or related disorders (e.g., Hyperinsulinism, insulin resistance and related disorders);
   ii) for preventing or reversing insulin resistance;
   iii) for reversing dysfunction and/or de-differentiation of pancreatic islet cells;
   iv) for increasing the number of pancreatic beta cells;
   v) for production or preparation of an antibody;
   vi) for immunizing a non-human animal;
   vii) for preparation of a hybridoma;
   viii) for immunoprecipitation (e.g., “pull-down” assay) and/or for identification of epitope or co-epitope or antigen by the means of one or more of the following: epitope mapping, cryo electron microscopy, protein crystallization, chemical crosslinking, mass spectrometry, HDX—exchange mass spectrometry, NMR and other suitable structural methods, phage display and/or similar methods;
   ix) in a method according to any one of preceding items;
   x) in any one of (i)-(ix), wherein said use is in vitro, ex vivo or in vivo.
64. The method or use according to any one of preceding items, wherein said diabetes comprises type 1 diabetes, type 2 diabetes, gestational diabetes, prediabetes, insulin resistance, metabolic syndrome or glucose tolerance.
65. A method of immunoprecipitation (e.g., IP or “pull-down” assay) comprising contacting an antibody of any one of the preceding items with a specific substance (e.g., a polypeptide, e.g., a recombinant polypeptide) from/in a solution.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., “about 20” includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be had from the following examples, offered for illustrative purposes only. The examples are not intended to limit the scope of the present invention in any way.

### EXAMPLES

### Materials and Methods

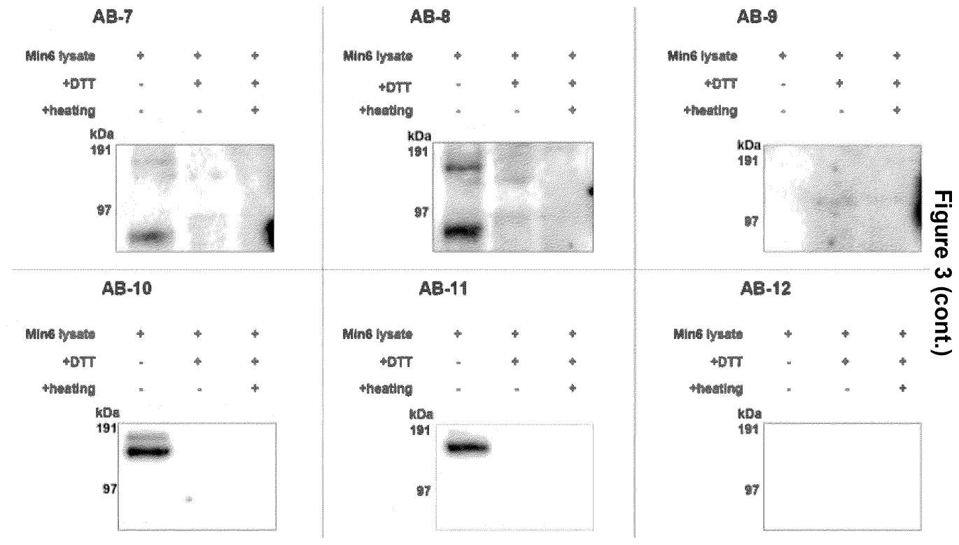

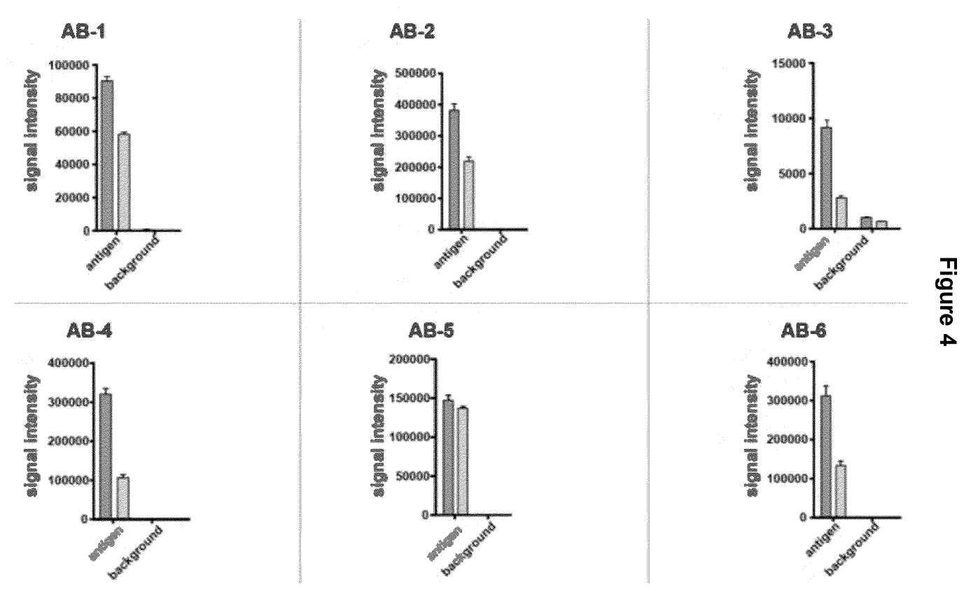

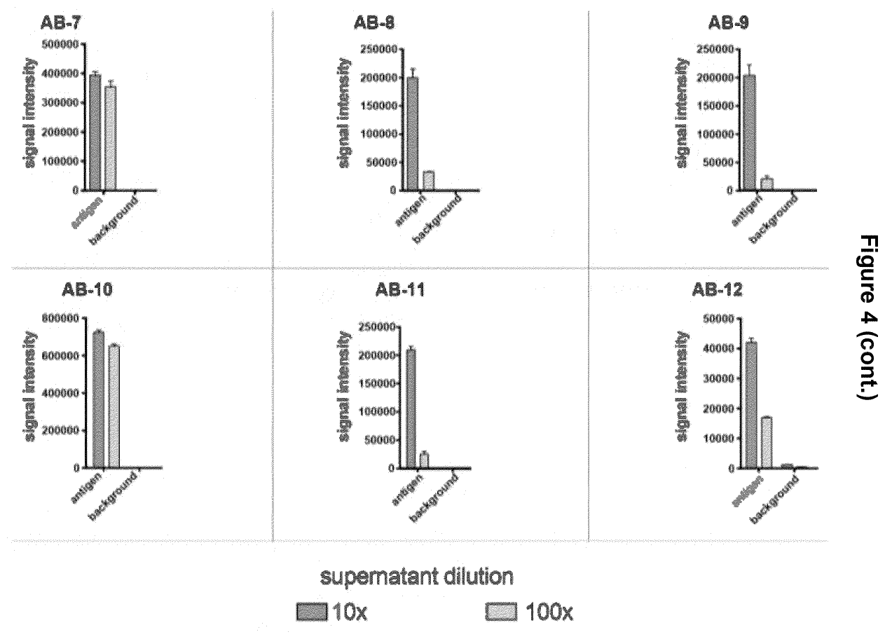

### Western Blot (e.g., as Shown in FIG. 2-3 Herein)

Min 6 cells were lysed (50 mM Tris-HCl, 150 mM NaCl, 2 mM EDTA, 1% NP-40, pH 8.0) and mixed (4:1) with loading buffer (250 mM Tris-HCl, 12.5 mM Ethylenediaminetetraacetic acid, 10% Sodium dodecyl sulfate, 25% glycerol). Where necessary 20 mM DTT and heating (95 deg, 5 min) were applied. Sodium dodecyl sulfate polyacrylamide gel electrophoresis (SDS-PAGE) of Min6 cell lysates was performed in NuPAGE gels (4-12% bis-tris gels, and MOPS buffer, Invitrogen). For Western blotting a 0.45 μm Immobilon-P PVDF membrane (Millipore) was used. Membrane blocking was carried out with 5% (w/v) milk (Carl Roth, Germany) in TBST (50 mM Tris-HCl, pH 7.4, 150 mM NaCl, and 0.05% Tween 20) for 1 h at room temperature. Monoclonal rat anti-IGFR1 antibodies (1 ug/mL in TBST+5% (w/v) milk) were applied for 1 h at RT, followed with 3 subsequent washes in TBST. The secondary antibody, peroxidase-conjugated mouse anti-rat (isotype specific; in TBST+5% milk), was incubated with the membrane for 1 h at RT. After three washes with TBST, HRP reaction was developed using West Femto substrate (Thermo Fisher Scientific) and detected using the LAS-3000 Imaging System (Fuji).

### Electrochemiluminescence-ELISA (e.g., as Shown in FIG. 4 Herein)

Antigens (1 ug) were passively adsorbed on the electrode surface (1 h, 23° C.), and the residual sites on the surface were blocked with 0.2% porcine gelatin (1 h, 23° C.). The surface was then washed three times with HBS (25 mM HEPES, 150 mM NaCl, pH 7.25). For the initial screening the supernatants collected from hybridoma cultures were diluted 10 and 100-fold with 0.2% porcine gelatin solutions.

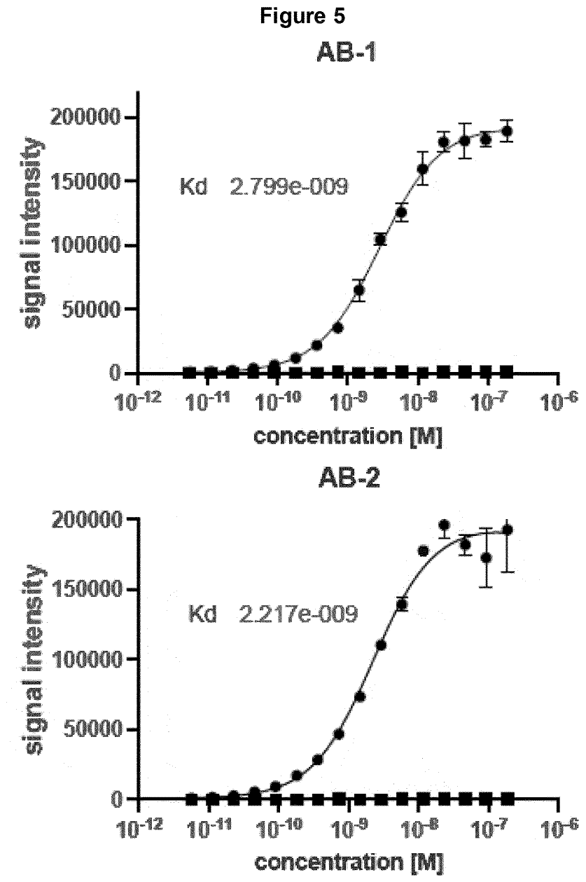

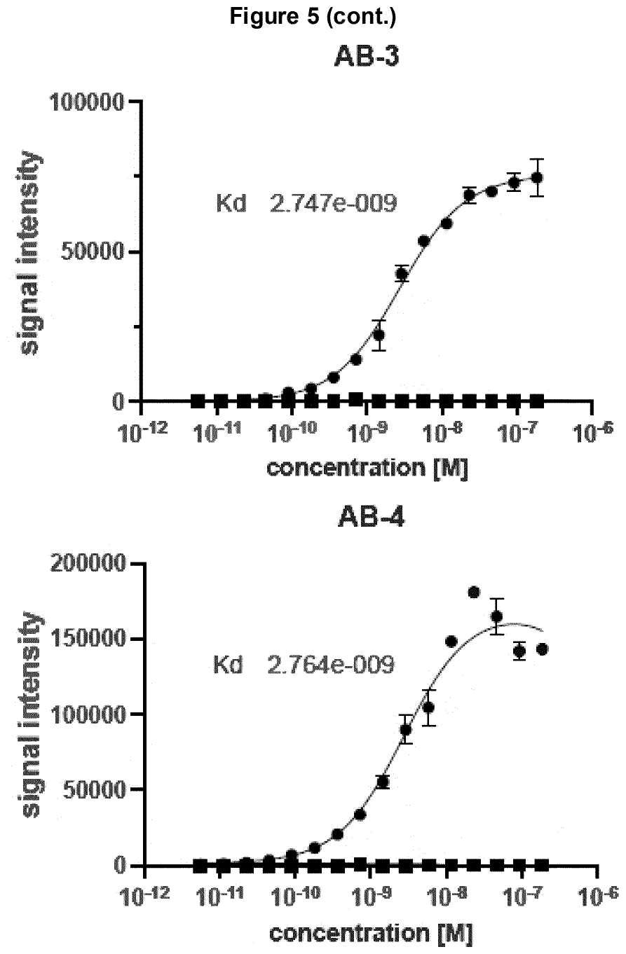

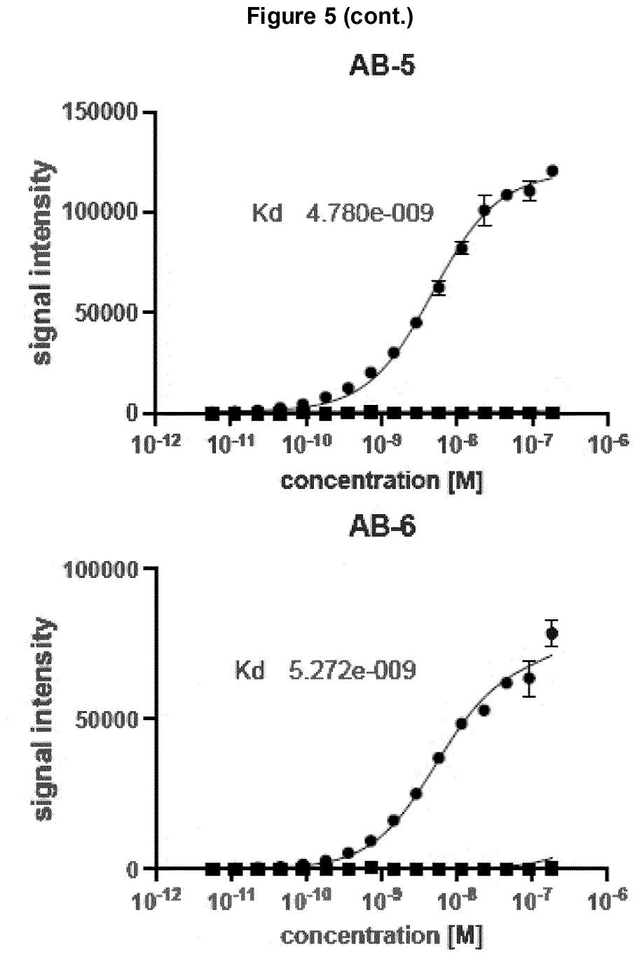

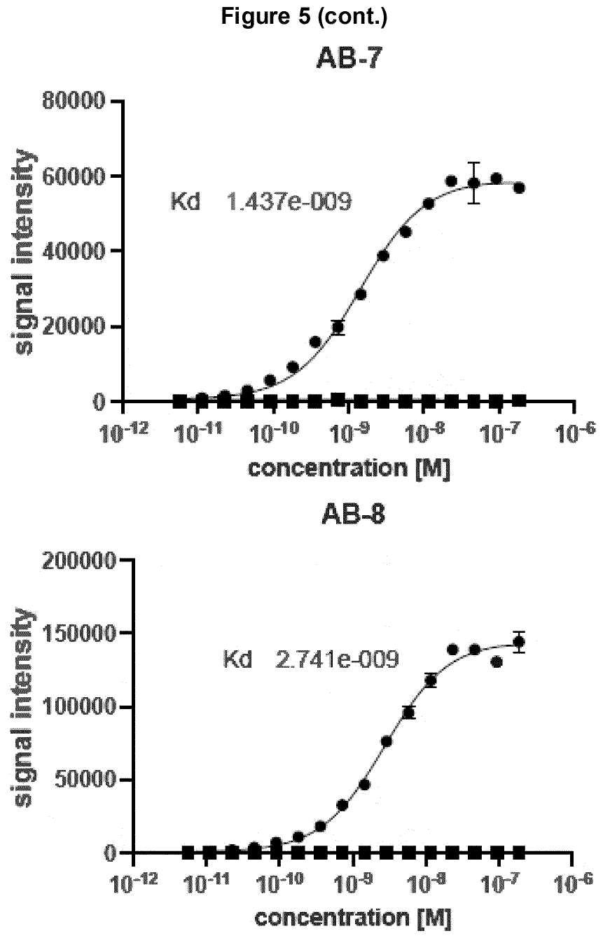

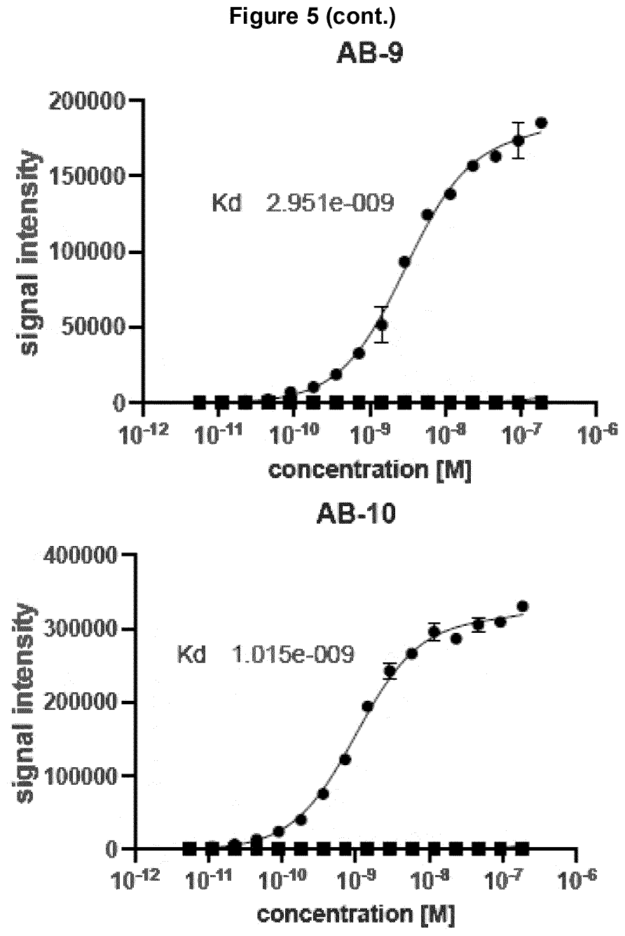

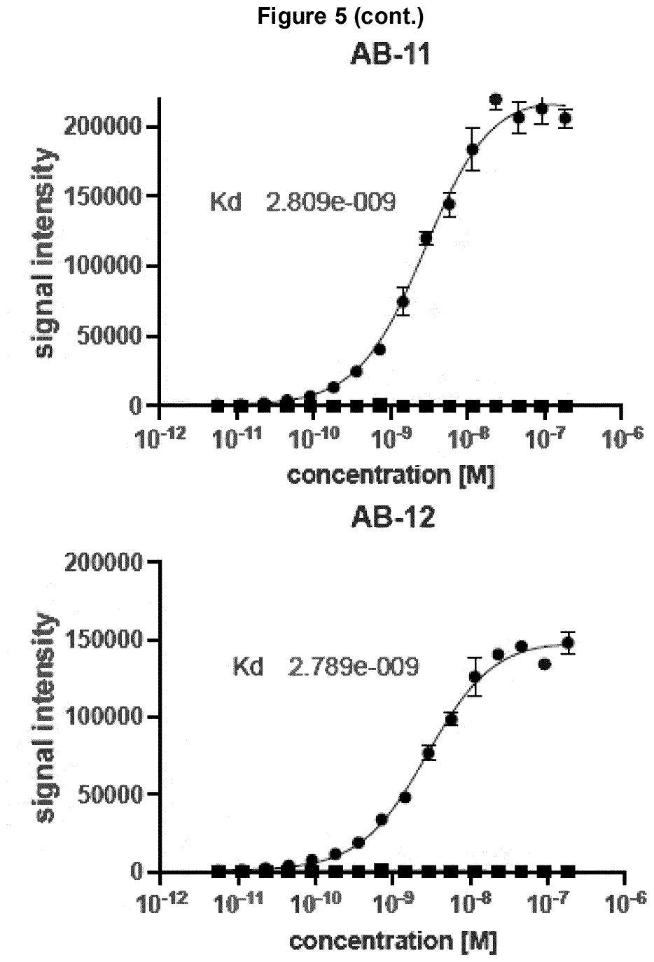

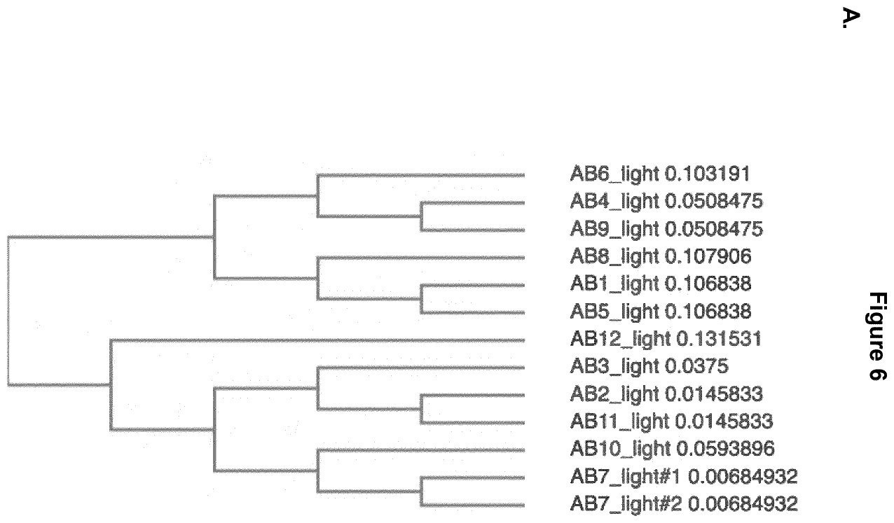

### K_{d} Measurements of Antibody Affinity (e.g., as Shown in FIG. 5 Herein)

K_{d} measurements of antibody affinity for AB1 to AB-12 were carried out according to standard protocol. In brief, for affinity measurements porcine gelatin solutions containing the desired concentrations of purified rat monoclonal anti-IGFR1 were added to each well. Binding was carried out for 2 h at 23° C. Plates were then washed 3× with HBS solution and a solution of isotype specific secondary anti rat detection antibody was added (1 μg/mL) and incubated (23° C., 1 h). The wells were washed 3× with HBS and reading buffer was added (MSD surfactant-free reading buffer). Data were acquired on a SECTOR Imager 6000 chemiluminescence reader. The background was determined from binding of secondary antibodies to antigen. The recorded data were analyzed using GraphPad Prism 6.0 software using one site-specific binding algorithm.

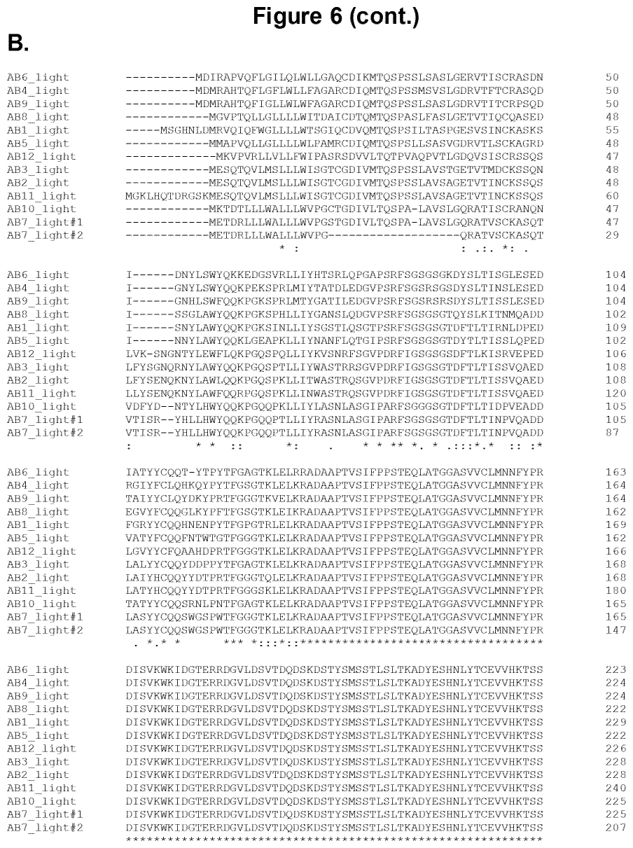

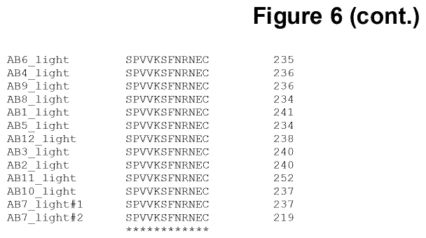

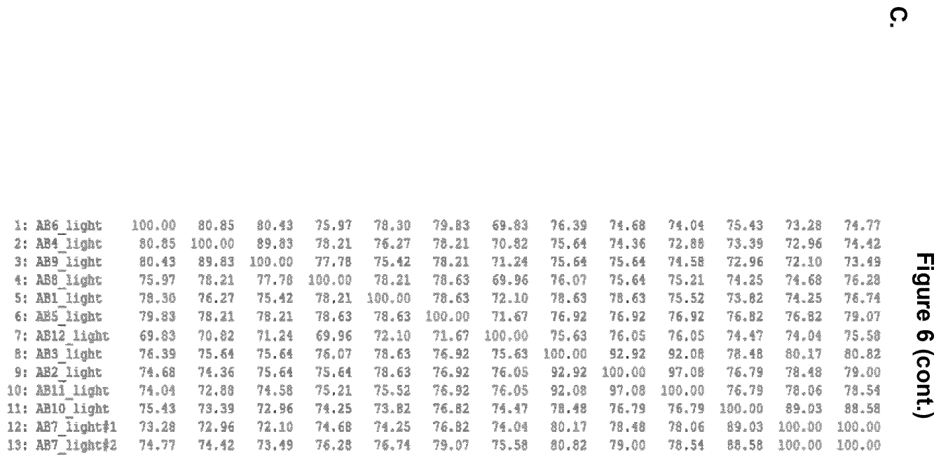

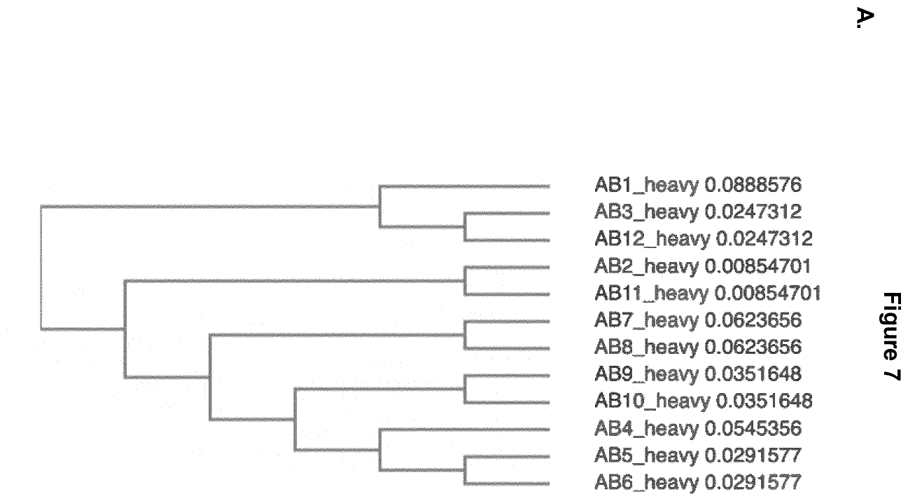

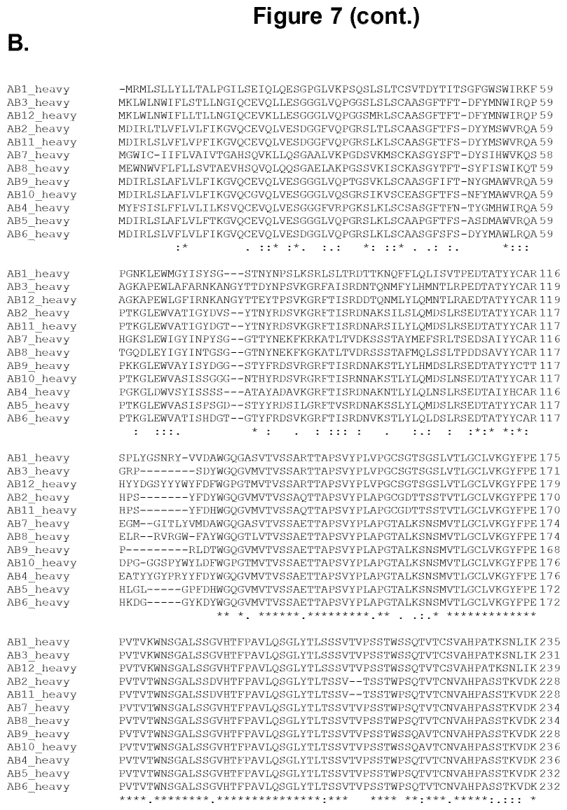

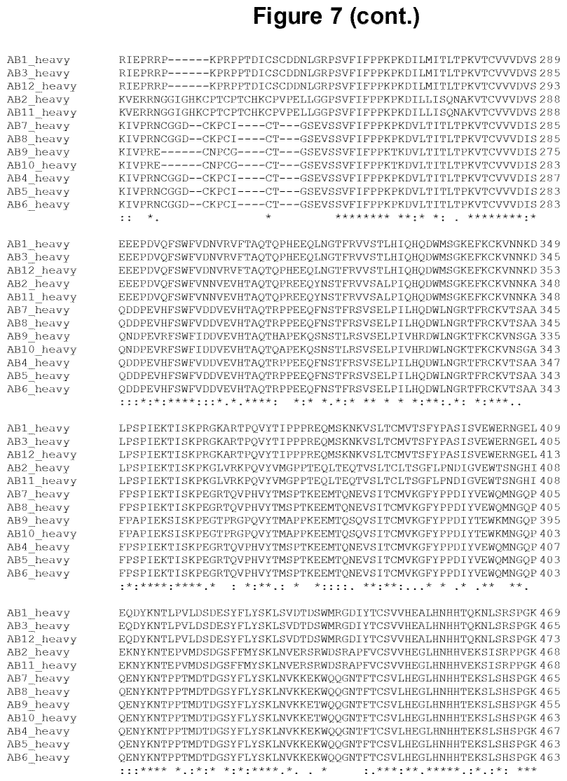

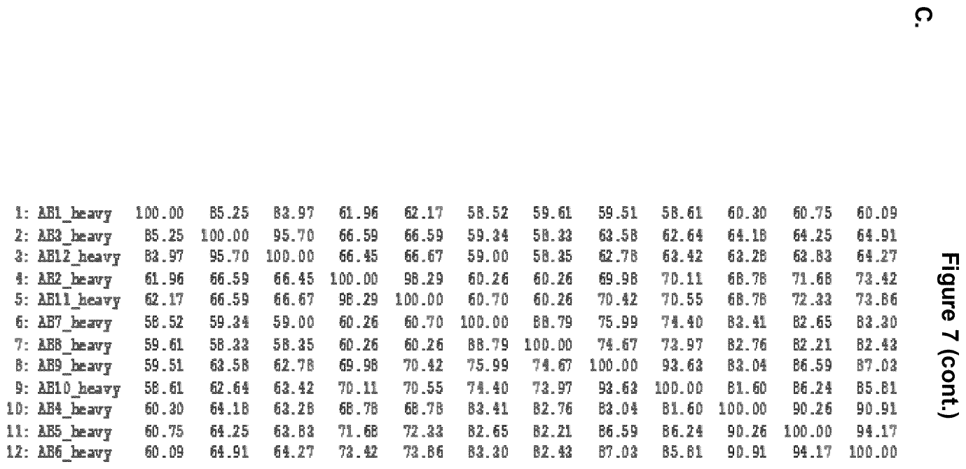

### Sequence Alignment (e.g., as Shown in FIGS. 6-7 Herein)

The alignments were performed using the Clustal Omega web service of the EMBL (https://www.ebi.ac.uk/Tools/msa/clustalo/), using the standard conditions i.e. with enabled mbed-like clustering guide tree and mbed-like clustering iterations.

Example 1: Comparison of the Unique Superior Properties of the Novel Structure-Specific Monoclonal Antibodies (AB) of the Present Invention to that of the Domain-Specific Peptide ABs from the Prior Art

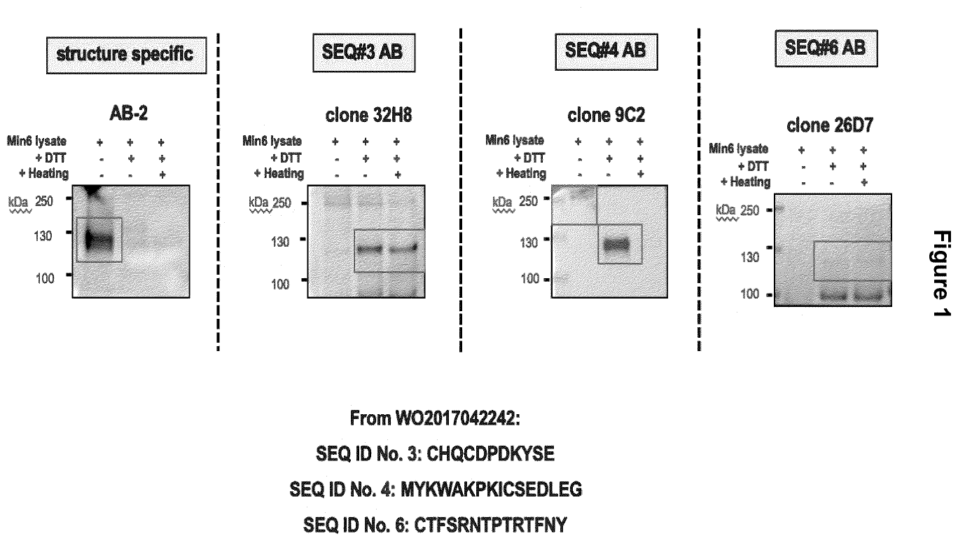

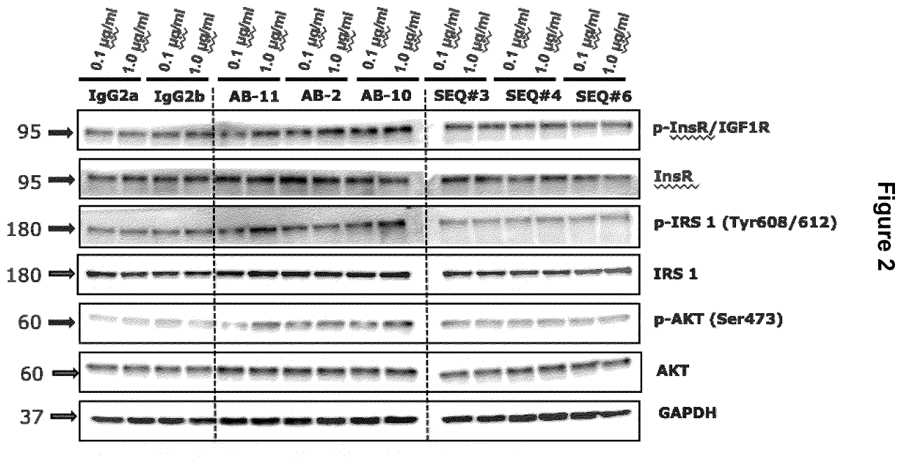

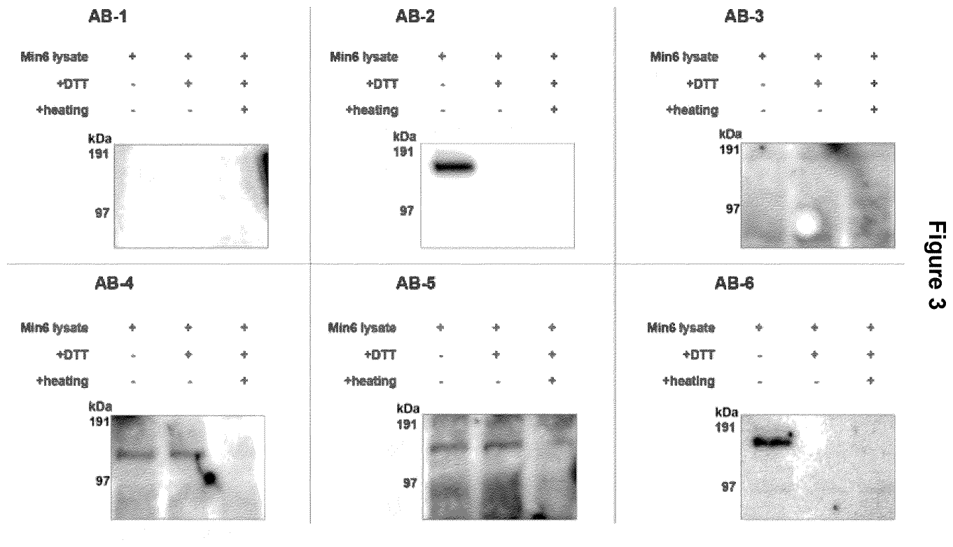

Methods (e.g., as used in FIG. 1): Min6 cells were lysed in mild lysis buffer and proteins were quantified using BCA assay. Min6 lysates were used for sample preparation in 3 different ways: a) Min6 lysate+4× Laemmli buffer without β-ME. No DTT was added and samples were not heat denatured. b) Min6 lysate+4× Laemmli buffer without β-ME. DTT was added to the loading dye. Samples were not heat denatured. c) Min6 lysate+4× Laemmli buffer without β-ME. DTT was added to the loading dye. Samples were heat denatured at 95° C. for 10 min. Samples were then loaded on 10% SDS gel for separation and Western blot analysis was performed. Primary antibodies were used at 1:1000 dilution in milk blocking solution overnight at 4° C. Respective secondary antibodies were added and signal was developed.

Results (as in FIG. 1): The structure-specific AB (exemplified by AB-2) of the present invention detects the protein under non-denaturing conditions in a SDS gel after Western blotting, whereas the peptide ABs from the prior art only detect the denatured protein (e.g., FIG. 1).

Methods (e.g., as used in FIG. 2): Min6 cells were cultured in a 6-well plate till they reached 80% confluency. On the day of experiment, cells were treated with AB and their respective isotype controls with 2 different concentrations (0.1 μg/ml and 1.0 μg/ml—final conc. in completed DMEM medium) for 15 min at 37° C. incubator. Cells were washed gently with ice-cold PBS and lysed in cold lysis buffer containing protease and phosphatase inhibitor cocktails (1:100). Cell lysate were collected in an Eppendorf tube and incubated in cold room on a rotor for 20-30 min to promote proper lysis of cells. Cell lysate were centrifuged at max. speed to remove cell debris. Supernatant was collected in a fresh tube and proteins were quantified using BCA assay. 15-20 μg proteins were loaded onto a 10% SDS gel for WB analysis.

Results (as in FIG. 2): Novel structure-specific AB of the present invention activates p-InsR/IGF1R, p-IRS1 and p-AKT in a dose-dependent manner, whereas domain-specific peptide antibodies from the prior art have no or inhibitory effects on InsR/IGF1R pathway activation.

### Example 2: Novel Antibodies are Capable of Modulating (e.g., Increasing) Insulin Receptor Sensitivity

Methods: Min6 cells were prepared shown in FIG. 8 (see also Example 1 for details) followed by Western blotting as described in Example 1 above.

### Results (as in FIG. 8):

All twelve antibodies of the present invention were generated using a specific method against the native extracellular domain of IGFR-L1/Inceptor. Inceptor binds indirectly to the insulin receptor complex at the plasma membrane and mediates internalization and thus desensitization of the activated insulin receptor complex. Consequently, when insulin-producing beta cells are incubated with any of the 12 antibodies of the present invention, the antibody binds to the extracellular domain of the inceptor and thus prevents interaction with the activated insulin receptor complex. Thus, the insulin receptor is not desensitized and insulin signal activation is enhanced and/or prolonged (FIG. 8). In other words, the Inceptor Ak of the present invention bind to Inceptor (they all might have a different paratope i.e. epitope) and thereby prevent the Inceptor from efficiently coming together with the activated insulin receptor complex. Thus, the insulin receptor is not efficiently desensitized and remains longer at the plasma membrane and is prolongly activated. This is supported by FIG. 8 for all 12 novel Aks of the present invention (p-IR/IGF1R) and provides a unifying feature for all Aks of the present invention, besides the unique production method.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied herein may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. An antibody which binds to the extracellular domain of folded (e.g., native) or partially folded (e.g., having a tertiary structure with or without post-translational modification) Inceptor protein comprising the amino acid sequence of SEQ ID NO: 1, preferably to a native epitope within IGFR-L1 protein, wherein binding to said folded or partially folded IGFR-L1 protein by the antibody is determinable (e.g., determined) as follows:
(a) incubating an aliquot of a lysate from Min6 cells (ATCC CRL-11506) separately with each of
(i) a buffer suitable for SDS-PAGE of proteins for 5 min at ambient temperature,
(ii) a buffer suitable for SDS-PAGE of proteins and a reducing agent for 5 min at ambient temperature, and
(iii) a buffer suitable for SDS-PAGE of proteins and a reducing agent for 5 min at 95° C.,
(b) subjecting said aliquots to SDS-PAGE;
(c) western blotting the SDS-PAGE gel;
(d) incubating the western blot membrane with the antibody;
wherein the antibody detects only aliquot (i).

2. The antibody of claim 1, wherein step (a) comprises
(a) incubating an aliquot of a lysate from Min6 cells (ATCC CRL-11506) separately with each of
(i) 50 mM Tris-HCl pH=6.8, 2.5 mM EDTA, 2% (w/v) SDS and 5% (v/v) glycerol, buffer without a thiol agent for 5 min at ambient temperature,
(ii) 50 mM Tris-HCl pH=6.8, 2.5 mM EDTA, 2% (w/v) SDS and 5% (v/v) glycerol, 20 mM DTT for 5 min at ambient temperature, and
(iii) 50 mM Tris-HCl pH=6.8, 2.5 mM EDTA, 2% (w/v) SDS and 5% (v/v) glycerol, 20 mM DTT for 5 min at 95° C.

3. The antibody of claim 1, wherein the antibody increases or decreases or modulates or inhibits binding of one or more growth factors (e.g., insulin) or similar ligand/s, morphogen/s, homo-oligomerization partner/s, hetero-oligomerization partner/s and/or di- or oligomerization partner/s to Inceptor protein and/or other receptor/s, preferably wherein the antibody increases or modulates insulin receptor sensitization.

4. The antibody of any one of the preceding claims, wherein the antibody increases InsR-mediated signaling.

5. The antibody of any one of the preceding claims, wherein the antibody increases phosphorylation of InsR, IRS1 and/or AKT.

6. The antibody of any one of the preceding claims, wherein said extracellular domain ranges from amino acids 1 to 910 of SEQ ID NO: 1, preferably said extracellular domain ranges from amino acids 42 to 910 of SEQ ID NO: 1.

7. The antibody of any one of the preceding claims, wherein the antibody binds to Inceptor protein with a Kd of 10 nM or less, preferably 6 nM or less, more preferably 5 nM or less, even more preferably 3 nM or less.

8. The antibody of any one of the preceding claims, wherein the antibody is producible by immunizing a rodent animal with proteoliposomes comprising Inceptor protein.

9. The antibody of any one of the preceding claims, wherein the antibody is
(a) an antibody comprising a heavy chain variable region having an amino acid sequence with at least 85% identity to the amino acids 19 to 140 of SEQ ID NO: 2 and a light chain variable region having an amino acid sequence with at least 85% identity to the amino acids 28 to 136 of SEQ ID NO: 12;
(b) an antibody comprising a heavy chain variable region having an amino acid sequence with at least 85% identity to the amino acids 62 to 177 of SEQ ID NO: 22 and a light chain variable region having an amino acid sequence with at least 85% identity to the amino acids 48 to 162 of SEQ ID NO: 32;
(c) an antibody comprising a heavy chain variable region having an amino acid sequence with at least 85% identity to the amino acids 20 to 136 of SEQ ID NO: 42 and a light chain variable region having an amino acid sequence with at least 85% identity to the amino acids 39 to 153 of SEQ ID NO: 52;
(d) an antibody comprising a heavy chain variable region having an amino acid sequence with at least 85% identity to the amino acids 20 to 141 of SEQ ID NO: 62 and a light chain variable region having an amino acid sequence with at least 85% identity to the amino acids 40 to 148 of SEQ ID NO: 72;
(e) an antibody comprising a heavy chain variable region having an amino acid sequence with at least 85% identity to the amino acids 58 to 175 of SEQ ID NO: 82 and a light chain variable region having an amino acid sequence with at least 85% identity to the amino acids 21 to 129 of SEQ ID NO: 92;
(f) an antibody comprising a heavy chain variable region having an amino acid sequence with at least 85% identity to the amino acids 54 to 171 of SEQ ID NO: 102 and a light chain variable region having an amino acid sequence with at least 85% identity to the amino acids 55 to 163 of SEQ ID NO: 112;
(g) an antibody comprising a heavy chain variable region having an amino acid sequence with at least 85% identity to the amino acids 19 to 139 of SEQ ID NO: 122 and a light chain variable region having an amino acid sequence with at least 85% identity to the amino acids 45 to 156 of SEQ ID NO: 132;
(h) an antibody comprising a heavy chain variable region having an amino acid sequence with at least 85% identity to the amino acids 20 to 139 of SEQ ID NO: 142 and a light chain variable region having an amino acid sequence with at least 85% identity to the amino acids 40 to 148 of SEQ ID NO: 152;
(i) an antibody comprising a heavy chain variable region having an amino acid sequence with at least 85% identity to the amino acids 20 to 133 of SEQ ID NO: 162 and a light chain variable region having an amino acid sequence with at least 85% identity to the amino acids 40 to 148 of SEQ ID NO: 172;
(j) an antibody comprising a heavy chain variable region having an amino acid sequence with at least 85% identity to the amino acids 54 to 175 of SEQ ID NO: 182 and a light chain variable region having an amino acid sequence with at least 85% identity to the amino acids 38 to 149 of SEQ ID NO: 192;
(k) an antibody comprising a heavy chain variable region having an amino acid sequence with at least 85% identity to the amino acids 55 to 170 of SEQ ID NO: 202 and a light chain variable region having an amino acid sequence with at least 85% identity to the amino acids 21 to 135 of SEQ ID NO: 212;
(l) an antibody comprising a heavy chain variable region having an amino acid sequence with at least 85% identity to the amino acids 20 to 144 of SEQ ID NO: 222 and a light chain variable region having an amino acid sequence with at least 85% identity to the amino acids 52 to 165 of SEQ ID NO: 232; or
(m) an antibody which binds to the same epitope as that in Inceptor protein to which any one of the antibodies (a) to (l) bind.

10. The antibody of any one of the preceding claims, wherein the antibody is
(a) an antibody comprising a heavy chain variable region comprising heavy chain CDR1 having the amino acid sequence as set forth in SEQ ID NO: 5, heavy chain CDR2 having the amino acid sequence as set forth in SEQ ID NO: 7, and heavy chain CDR3 having the amino acid sequence as set forth in SEQ ID NO: 9 and
a light chain variable region comprising light chain CDR1 having the amino acid sequence as set forth in SEQ ID NO: 15, light chain CDR2 having the amino acid sequence as set forth in SEQ ID NO: 17, and light chain CDR3 having the amino acid sequence as set forth in SEQ ID NO: 19;
(b) an antibody comprising a heavy chain variable region comprising heavy chain CDR1 having the amino acid sequence as set forth in SEQ ID NO: 25, heavy chain CDR2 having the amino acid sequence as set forth in SEQ ID NO: 27, and heavy chain CDR3 having the amino acid sequence as set forth in SEQ ID NO: 29, and
a light chain variable region comprising light chain CDR1 having the amino acid sequence as set forth in SEQ ID NO: 35, light chain CDR2 having the amino acid sequence as set forth in SEQ ID NO: 37, and light chain CDR3 having the amino acid sequence as set forth in SEQ ID NO: 39;
(c) an antibody comprising a heavy chain variable region comprising heavy chain CDR1 having the amino acid sequence as set forth in SEQ ID NO: 45, heavy chain CDR2 having the amino acid sequence as set forth in SEQ ID NO: 47, and heavy chain CDR3 having the amino acid sequence as set forth in SEQ ID NO: 49, and
a light chain variable region comprising light chain CDR1 having the amino acid sequence as set forth in SEQ ID NO: 55, light chain CDR2 having the amino acid sequence as set forth in SEQ ID NO: 57, and light chain CDR3 having the amino acid sequence as set forth in SEQ ID NO: 59;
(d) an antibody comprising a heavy chain variable region comprising heavy chain CDR1 having the amino acid sequence as set forth in SEQ ID NO: 65, heavy chain CDR2 having the amino acid sequence as set forth in SEQ ID NO: 67, and heavy chain CDR3 having the amino acid sequence as set forth in SEQ ID NO: 69, and
a light chain variable region comprising light chain CDR1 having the amino acid sequence as set forth in SEQ ID NO: 75, light chain CDR2 having the amino acid sequence as set forth in SEQ ID NO: 77, and light chain CDR3 having the amino acid sequence as set forth in SEQ ID NO: 79;
(e) an antibody comprising a heavy chain variable region comprising heavy chain CDR1 having the amino acid sequence as set forth in SEQ ID NO: 85, heavy chain CDR2 having the amino acid sequence as set forth in SEQ ID NO: 87, and heavy chain CDR3 having the amino acid sequence as set forth in SEQ ID NO: 89, and
a light chain variable region comprising light chain CDR1 having the amino acid sequence as set forth in SEQ ID NO: 95, light chain CDR2 having the amino acid sequence as set forth in SEQ ID NO: 97, and light chain CDR3 having the amino acid sequence as set forth in SEQ ID NO: 99;
(f) an antibody comprising a heavy chain variable region comprising heavy chain CDR1 having the amino acid sequence as set forth in SEQ ID NO: 105, heavy chain CDR2 having the amino acid sequence as set forth in SEQ ID NO: 107, and heavy chain CDR3 having the amino acid sequence as set forth in SEQ ID NO: 109, and
a light chain variable region comprising light chain CDR1 having the amino acid sequence as set forth in SEQ ID NO: 115, light chain CDR2 having the amino acid sequence as set forth in SEQ ID NO: 117, and light chain CDR3 having the amino acid sequence as set forth in SEQ ID NO: 119;
(g) an antibody comprising a heavy chain variable region comprising heavy chain CDR1 having the amino acid sequence as set forth in SEQ ID NO: 125, heavy chain CDR2 having the amino acid sequence as set forth in SEQ ID NO: 127, and heavy chain CDR3 having the amino acid sequence as set forth in SEQ ID NO: 129, and
a light chain variable region comprising light chain CDR1 having the amino acid sequence as set forth in SEQ ID NO: 135, light chain CDR2 having the amino acid sequence as set forth in SEQ ID NO: 137, and light chain CDR3 having the amino acid sequence as set forth in SEQ ID NO: 139;
(i) an antibody comprising a heavy chain variable region comprising heavy chain CDR1 having the amino acid sequence as set forth in SEQ ID NO: 145, heavy chain CDR2 having the amino acid sequence as set forth in SEQ ID NO: 147, and heavy chain CDR3 having the amino acid sequence as set forth in SEQ ID NO: 149, and
a light chain variable region comprising light chain CDR1 having the amino acid sequence as set forth in SEQ ID NO: 155, light chain CDR2 having the amino acid sequence as set forth in SEQ ID NO: 157, and light chain CDR3 having the amino acid sequence as set forth in SEQ ID NO: 159;
(i) an antibody comprising a heavy chain variable region comprising heavy chain CDR1 having the amino acid sequence as set forth in SEQ ID NO: 165, heavy chain CDR2 having the amino acid sequence as set forth in SEQ ID NO: 167, and heavy chain CDR3 having the amino acid sequence as set forth in SEQ ID NO: 169, and
a light chain variable region comprising light chain CDR1 having the amino acid sequence as set forth in SEQ ID NO: 175, light chain CDR2 having the amino acid sequence as set forth in SEQ ID NO: 177, and light chain CDR3 having the amino acid sequence as set forth in SEQ ID NO: 179;
(j) an antibody comprising a heavy chain variable region comprising heavy chain CDR1 having the amino acid sequence as set forth in SEQ ID NO: 185, heavy chain CDR2 having the amino acid sequence as set forth in SEQ ID NO: 187, and heavy chain CDR3 having the amino acid sequence as set forth in SEQ ID NO: 189, and
a light chain variable region comprising light chain CDR1 having the amino acid sequence as set forth in SEQ ID NO: 195, light chain CDR2 having the amino acid sequence as set forth in SEQ ID NO: 197, and light chain CDR3 having the amino acid sequence as set forth in SEQ ID NO: 199;
(k) an antibody comprising a heavy chain variable region comprising heavy chain CDR1 having the amino acid sequence as set forth in SEQ ID NO: 205, heavy chain CDR2 having the amino acid sequence as set forth in SEQ ID NO: 207, and heavy chain CDR3 having the amino acid sequence as set forth in SEQ ID NO: 209, and
a light chain variable region comprising light chain CDR1 having the amino acid sequence as set forth in SEQ ID NO: 215, light chain CDR2 having the amino acid sequence as set forth in SEQ ID NO: 217, and light chain CDR3 having the amino acid sequence as set forth in SEQ ID NO: 219;
(l) an antibody comprising a heavy chain variable region comprising heavy chain CDR1 having the amino acid sequence as set forth in SEQ ID NO: 225, heavy chain CDR2 having the amino acid sequence as set forth in SEQ ID NO: 227, and heavy chain CDR3 having the amino acid sequence as set forth in SEQ ID NO: 229, and
a light chain variable region comprising light chain CDR1 having the amino acid sequence as set forth in SEQ ID NO: 235, light chain CDR2 having the amino acid sequence as set forth in SEQ ID NO: 237, and light chain CDR3 having the amino acid sequence as set forth in SEQ ID NO: 239; or
(m) an antibody which binds to the same epitope as that in the human PRDX4 protein to which any one of the antibodies (a) to (l) bind.

11. The antibody of any one of the preceding claims, wherein said antibody is for use as a medicament and/or in therapy.

12. The antibody of any one of the preceding claims, wherein said antibody is for use in a method for:
i) treating diabetes, hyperinsulinism and/or insulin resistance and/or cancer; and/or
ii) for immunoprecipitation.

13. The antibody for the use of claim 12, wherein diabetes comprises type 1 diabetes, type 2 diabetes, gestational diabetes, prediabetes, insulin resistance, metabolic syndrome or glucose tolerance.

14. The antibody of any one of claims 1 to 11, wherein said antibody is for use in a method of preventing or reversing insulin resistance.

15. The antibody of any one of claims 1 to 11, wherein said antibody is for use in a method of reversing dysfunction and/or de-differentiation of pancreatic islet cells.

16. The antibody of any one of claims 1 to 11, wherein said antibody is for use in a method of increasing the number of pancreatic beta cells.
